SEQUENCE LISTING (86 sequences)

SEQ ID NO: 1 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 2 - protein, 133 aa, Homo sapiens [sequence omitted]

SEQ ID NO: 3 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 257 aa, Artificial Sequence
  Description of Artificial Sequence Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSEVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWNGYYSTHDFDSWGQGTTVTVSS

SEQ ID NO: 5 - protein, 121 aa, Artificial Sequence [sequence omitted]

SEQ ID NO: 6 - protein, 10 aa, Artificial Sequence
  Description of Artificial Sequence Synthetic peptide
GGGGSGGGGS

SEQ ID NO: 7 - protein, 126 aa, Artificial Sequence
  Description of Artificial Sequence Synthetic polypeptide
EVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWNGYYSTHDFDSWGQGTTVTVSS

SEQ ID NO: 8 - protein, 330 aa, Artificial Sequence [sequence omitted]

SEQ ID NO: 9 - protein, 225 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRGGSGGGGSGEIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKR

SEQ ID NO: 10 - protein, 108 aa, Artificial Sequence
  Description of Artificial Sequence Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 11 - protein, 9 aa, Artificial Sequence
  Description of Artificial Sequence Synthetic peptide
GGSGGGGSG

SEQ ID NO: 12 - protein, 108 aa, Artificial Sequence
  Description of Artificial Sequence Synthetic polypeptide
EIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKR

SEQ ID NO: 13 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 6 aa, Artificial Sequence
  Description of Artificial Sequence Synthetic peptide
GGGGSG

SEQ ID NO: 15 - protein, 5 aa, Artificial Sequence
  Description of Artificial Sequence Synthetic peptide
GGSGG

SEQ ID NO: 16 - protein, 10 aa, Artificial Sequence
  Description of Artificial Sequence Synthetic peptide
GGGGSGGGGS

SEQ ID NO: 17 - protein, 9 aa, Artificial Sequence
  Description of Artificial Sequence Synthetic peptide
GGSGGGGSG

SEQ ID NO: 18 - protein, 10 aa, Artificial Sequence
  Description of Artificial Sequence Synthetic peptide
GGSGGGGSGS

SEQ ID NO: 19 - protein, 13 aa, Artificial Sequence
  Description of Artificial Sequence Synthetic peptide
GGSGGGGSGGGGS

SEQ ID NO: 20 - protein, 14 aa, Artificial Sequence
  Description of Artificial Sequence Synthetic peptide
GGGGSGGGGSGGGG

SEQ ID NO: 21 - protein, 15 aa, Artificial Sequence
  Description of Artificial Sequence Synthetic peptide
GGGGSGGGGSGGGGS

SEQ ID NO: 22 - protein, 6 aa, Artificial Sequence
  Description of Artificial Sequence Synthetic peptide
ASTKGP

SEQ ID NO: 23 - protein, 13 aa, Artificial Sequence
  Description of Artificial Sequence Synthetic peptide
ASTKGPSVFPLAP

SEQ ID NO: 24 - protein, 5 aa, Artificial Sequence
  Description of Artificial Sequence Synthetic peptide
TVAAP

SEQ ID NO: 25 - protein, 6 aa, Artificial Sequence
  Description of Artificial Sequence Synthetic peptide
RTVAAP

SEQ ID NO: 26 - protein, 12 aa, Artificial Sequence
  Description of Artificial Sequence Synthetic peptide
TVAAPSVFIFPP

SEQ ID NO: 27 - protein, 13 aa, Artificial Sequence
  Description of Artificial Sequence Synthetic peptide
RTVAAPSVFIFPP

SEQ ID NO: 28 - protein, 16 aa, Artificial Sequence
  Description of Artificial Sequence Synthetic peptide
AKTTPKLEEGEFSEAR

SEQ ID NO: 29 - protein, 17 aa, Artificial Sequence
  Description of Artificial Sequence Synthetic peptide
AKTTPKLEEGEFSEARV

SEQ ID NO: 30 - protein, 9 aa, Artificial Sequence
  Description of Artificial Sequence Synthetic peptide
AKTTPKLGG

SEQ ID NO: 31 - protein, 10 aa, Artificial Sequence
  Description of Artificial Sequence Synthetic peptide
SAKTTPKLGG

SEQ ID NO: 32 - protein, 6 aa, Artificial Sequence
  Description of Artificial Sequence Synthetic peptide
SAKTTP

SEQ ID NO: 33 - protein, 6 aa, Artificial Sequence
  Description of Artificial Sequence Synthetic peptide
RADAAP

SEQ ID NO: 34 - protein, 9 aa, Artificial Sequence
  Description of Artificial Sequence Synthetic peptide
RADAAPTVS

SEQ ID NO: 35 - protein, 12 aa, Artificial Sequence
  Description of Artificial Sequence Synthetic peptide
RADAAAAGGPGS

SEQ ID NO: 36 - protein, 27 aa, Artificial Sequence
  Description of Artificial Sequence Synthetic peptide
RADAAAAGGGGSGGGGSGGGGSGGGGS

SEQ ID NO: 37 - protein, 18 aa, Artificial Sequence
  Description of Artificial Sequence Synthetic peptide
SAKTTPKLEEGEFSEARV

SEQ ID NO: 38 - protein, 5 aa, Artificial Sequence
  Description of Artificial Sequence Synthetic peptide
ADAAP

SEQ ID NO: 39 - protein, 12 aa, Artificial Sequence
  Description of Artificial Sequence Synthetic peptide
ADAAPTVSIFPP

SEQ ID NO: 40 - protein, 6 aa, Artificial Sequence
  Description of Artificial Sequence Synthetic peptide
QPKAAP

SEQ ID NO: 41 - protein, 13 aa, Artificial Sequence
  Description of Artificial Sequence Synthetic peptide
QPKAAPSVTLFPP

SEQ ID NO: 42 - protein, 6 aa, Artificial Sequence
  Description of Artificial Sequence Synthetic peptide
AKTTPP

SEQ ID NO: 43 - protein, 13 aa, Artificial Sequence
  Description of Artificial Sequence Synthetic peptide
AKTTPPSVTPLAP

SEQ ID NO: 44 - protein, 6 aa, Artificial Sequence
  Description of Artificial Sequence Synthetic peptide
AKTTAP

SEQ ID NO: 45 - protein, 13 aa, Artificial Sequence
  Description of Artificial Sequence Synthetic peptide
AKTTAPSVYPLAP

SEQ ID NO: 46 - protein, 15 aa, Artificial Sequence
  Description of Artificial Sequence Synthetic peptide
GENKVEYAPALMALS

SEQ ID NO: 47 - protein, 4 aa, Artificial Sequence
  Description of Artificial Sequence Synthetic peptide
FGXG

SEQ ID NO: 48 - protein, 9 aa, Artificial Sequence
  Description of Artificial Sequence Synthetic peptide
CXXXXXXXX

SEQ ID NO: 49 - protein, 15 aa, Artificial Sequence
  Description of Artificial Sequence Synthetic peptide
GPAKELTPLKEAKVS

SEQ ID NO: 50 - protein, 15 aa, Artificial Sequence
  Description of Artificial Sequence Synthetic peptide
GHEAAAVMQVQYPAS

SEQ ID NO: 51 - protein, 5 aa, Artificial Sequence
  Description of Artificial Sequence Synthetic peptide
LEWIG

SEQ ID NO: 52 - protein, 4 aa, Artificial Sequence
  Description of Artificial Sequence Synthetic peptide
WGXG

SEQ ID NO: 53 - protein, 5 aa, Artificial Sequence
  Description of Artificial Sequence Synthetic peptide
DYAMH

SEQ ID NO: 54 - protein, 17 aa, Artificial Sequence
  Description of Artificial Sequence Synthetic peptide
AITWNSGHIDYADSVEG

SEQ ID NO: 55 - protein, 12 aa, Artificial Sequence
  Description of Artificial Sequence Synthetic peptide
VSYLSTASSLDY

SEQ ID NO: 56 - protein, 5 aa, Artificial Sequence
  Description of Artificial Sequence Synthetic peptide
GYGIG

SEQ ID NO: 57 - protein, 17 aa, Artificial Sequence
  Description of Artificial Sequence Synthetic peptide
GITPFFGFADYAQKFQG

SEQ ID NO: 58 - protein, 17 aa, Artificial Sequence
  Description of Artificial Sequence Synthetic peptide
DPNEFWNGYYSTHDFDS

SEQ ID NO: 59 - protein, 11 aa, Artificial Sequence
  Description of Artificial Sequence Synthetic peptide
RASQGIRNYLA

SEQ ID NO: 60 - protein, 7 aa, Artificial Sequence
  Description of Artificial Sequence Synthetic peptide
AASTLQS

SEQ ID NO: 61 - protein, 9 aa, Artificial Sequence
  Description of Artificial Sequence Synthetic peptide
QRYNRAPYT

SEQ ID NO: 62 - protein, 11 aa, Artificial Sequence
  Description of Artificial Sequence Synthetic peptide
RASQDIGSELH

SEQ ID NO: 63 - protein, 7 aa, Artificial Sequence
  Description of Artificial Sequence Synthetic peptide
YASHSTS

SEQ ID NO: 64 - protein, 9 aa, Artificial Sequence
  Description of Artificial Sequence Synthetic peptide
HQTDSLPYT

SEQ ID NO: 65 - protein, 5 aa, Artificial Sequence
  Description of Artificial Sequence Synthetic peptide
NYGII

SEQ ID NO: 66 - protein, 17 aa, Artificial Sequence
  Description of Artificial Sequence Synthetic peptide
WINTYTGKPTYAQKFQG

SEQ ID NO: 67 - protein, 15 aa, Artificial Sequence
  Description of Artificial Sequence Synthetic peptide
KLFTTMDVTDNAMDY

SEQ ID NO: 68 - protein, 5 aa, Artificial Sequence
  Description of Artificial Sequence Synthetic peptide
DYEIH

SEQ ID NO: 69 - protein, 255 aa, Artificial Sequence
  Description of Artificial Sequence Synthetic polypeptide
EVQLVQSGAEVKKPGASVKVSCKASGYTFANYGIIWVRQAPGQGLEWMGWINTYTGKPTYAQKFQGRVTMTTDTSTSTAYMELSSLRSEDTAVYYCARKLFTTMDVTDNAMDYWGQGTTVTVSSGGGGSGGGGSEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 70 - protein, 124 aa, Artificial Sequence
  Description of Artificial Sequence Synthetic polypeptide
EVQLVQSGAEVKKPGASVKVSCKASGYTFANYGIIWVRQAPGQGLEWMGWINTYTGKPTYAQKFQGRVTMTTDTSTSTAYMELSSLRSEDTAVYYCARKLFTTMDVTDNAMDYWGQGTTVTVSS

SEQ ID NO: 71 - protein, 10 aa, Artificial Sequence
  Description of Artificial Sequence Synthetic peptide
GGGGSGGGGS

SEQ ID NO: 72 - protein, 121 aa, Artificial Sequence
  Description of Artificial Sequence Synthetic polypeptide
EVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 73 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 225 aa, Artificial Sequence
  Description of Artificial Sequence Synthetic polypeptide
DIQMTQSPSSLSASVGDRVTITCRASQDISQYLNWYQQKPGKAPKLLIYYTSRLQSGVPSRFSGSGSGTDFTLTISSLQPEDFATYFCQQGNTWPPTFGQGTKLEIKRGGSGGGGSGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 75 - protein, 108 aa, Artificial Sequence
  Description of Artificial Sequence Synthetic polypeptide
DIQMTQSPSSLSASVGDRVTITCRASQDISQYLNWYQQKPGKAPKLLIYYTSRLQSGVPSRFSGSGSGTDFTLTISSLQPEDFATYFCQQGNTWPPTFGQGTKLEIKR

SEQ ID NO: 76 - protein, 9 aa, Artificial Sequence
  Description of Artificial Sequence Synthetic peptide
GGSGGGGSG

SEQ ID NO: 77 - protein, 108 aa, Artificial Sequence
  Description of Artificial Sequence Synthetic polypeptide
DIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 78 - protein, 106 aa, Artificial Sequence; identical to SEQ ID NO: 13
  Description of Artificial Sequence Synthetic polypeptide

SEQ ID NO: 79 - protein, 17 aa, Artificial Sequence
  Description of Artificial Sequence Synthetic peptide
VNDPESGGTFYNQKFDG

SEQ ID NO: 80 - protein, 12 aa, Artificial Sequence
  Description of Artificial Sequence Synthetic peptide
YSKWDSFDGMDY

SEQ ID NO: 81 - protein, 11 aa, Artificial Sequence
  Description of Artificial Sequence Synthetic peptide
RASQDISQYLN

SEQ ID NO: 82 - protein, 7 aa, Artificial Sequence
  Description of Artificial Sequence Synthetic peptide
YTSRLQS

SEQ ID NO: 83 - protein, 9 aa, Artificial Sequence
  Description of Artificial Sequence Synthetic peptide
QQGNTWPPT

SEQ ID NO: 84 - protein, 11 aa, Artificial Sequence
  Description of Artificial Sequence Synthetic peptide
RASSGIISYID

SEQ ID NO: 85 - protein, 7 aa, Artificial Sequence
  Description of Artificial Sequence Synthetic peptide
ATFDLAS

SEQ ID NO: 86 - protein, 9 aa, Artificial Sequence
  Description of Artificial Sequence Synthetic peptide
RQVGSYPET

(19) United States
(12) Patent Application Publication
(10) Pub. No.: US 2015/0291689 A1
(43) Pub. Date: Oct. 15, 2015

(54) Compositions and Methods for Treating Rheumatoid Arthritis

(71) Applicant: AbbVie, Inc., North Chicago, IL (US)

(72) Inventors: Robert J. Padley, Lake Bluff, IL (US); Renee Heuser, Lindenhurst, IL (US)

(21) Appl. No.: 14/642,502

(22) Filed: Mar. 9, 2015

Related U.S. Application Data
(60) Provisional application No. 61/950,172, filed on Mar. 9, 2014.
(60) Provisional application No. 61/950,515, filed on Mar. 10, 2014.
(60) Provisional application No. 62/010,868, filed on Jun. 11, 2014.
(60) Provisional application No. 62/016,067, filed on Jun. 23, 2014.
(60) Provisional application No. 62/065,498, filed on Oct. 17, 2014.
(60) Provisional application No. 62/080,108, filed on Nov. 14, 2014.
(60) Provisional application No. 62/109,452, filed on Jan. 29, 2015.
(60) Provisional application No. 62/130,362, filed on Mar. 9, 2015.

(51) Int. Cl. C07K 16/24; A61K 39/395; A61K 31/519; A61K 45/06

(52) U.S. Cl. CPC C07K 16/244; A61K 45/06; C07K 16/241; A61K 39/3955; A61K 31/519; C07K 2317/31; C07K 2317/565; A61K 2039/505

(57) ABSTRACT

Proteins that bind both IL-17 and TNF are described along with their use in compositions and methods for treating, preventing, and ameliorating rheumatoid arthritis.

### RELATED APPLICATIONS

The instant application claims the benefit of priority to U.S. provisional application Ser. No. 62/130,362 filed Mar. 9, 2015; U.S. provisional application Ser. No. 62/109,452 filed Jan. 29, 2015; U.S. provisional application Ser. No. 62/080,108 filed Nov. 14, 2014; U.S. provisional application Ser. No. 62/065,498 filed Oct. 17, 2014; U.S. provisional application Ser. No. 62/016,067 filed Jun. 23, 2014; U.S. provisional application Ser. No. 62/010,868 filed Jun. 11, 2014; U.S. provisional application Ser. No. 61/950,515 filed Mar. 10, 2014; and U.S. provisional application Ser. No. 61/950,172 filed Mar. 9, 2014, the contents of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on May 21, 2015, is named 566019_BBI-351_SL.txt and is 47,104 bytes in size.

### FIELD OF THE INVENTION

The present invention relates to bispecific TNF and IL-17 binding proteins, and to their uses in the prevention and/or treatment of rheumatoid arthritis.

### BACKGROUND OF THE INVENTION

Rheumatoid arthritis (RA) is an autoimmune disease that affects over a million Americans, with a significantly higher occurrence among women than men. Disease-modifying anti-rheumatic drugs (DMARDs) are often used to control the progression of RA and to try to prevent joint deterioration and disability. However, DMARD resistance occurs in some patients, for example, those who are receiving the DMARD methotrexate.

In addition to traditional DMARD therapies, a number of biologic therapies that target pro-inflammatory mediators, such as tumor necrosis factor-α, have been used successfully. Tumor necrosis factor-α (TNF-α) is a multifunctional pro-inflammatory cytokine secreted predominantly by monocytes/macrophages that also has effects on lipid metabolism, coagulation, insulin resistance, and endothelial function. TNF-α triggers pro-inflammatory pathways that result in tissue injury, such as degradation of cartilage and bone, induction of adhesion molecules, induction of pro-coagulant activity on vascular endothelial cells, an increase in the adherence of neutrophils and lymphocytes, and stimulation of the release of platelet activating factor from macrophages, neutrophils and vascular endothelial cells. Since TNF-α contributes to the etiology of many inflammatory disorders, including RA, it is a useful target for specific immunotherapy.

Adalimumab (also known by its trademark HUMIRA®) is a recombinant human monoclonal antibody specific for TNF-α. This monoclonal antibody binds to TNF-α and blocks its interaction with the p55 and p75 cell-surface TNF-α receptors. See, U.S. Pat. No. 6,090,382, the entire teaching of which is incorporated herein by reference. Adalimumab is used to treat a number of inflammatory disorders such as rheumatoid arthritis. Although adalimumab and other TNF-α inhibitors have revolutionized RA therapy, a significant portion of patients do not respond adequately to these drugs.

Interleukin-17A (IL-17A) is an inflammatory cytokine produced by T_{H}17 T cells that contributes to the etiology of a number of inflammatory diseases, including RA. IL-17A may exist as either a homodimer or as a heterodimer complexed with its homolog IL-17F to form heterodimeric IL-17A/F. IL-17A and IL-17F share 55% amino acid identity and bind to the same receptor (IL-17R), which is expressed on a wide variety of cells including vascular endothelial cells, peripheral T cells, B cells, fibroblast, lung cells, myelomonocytic cells, and marrow stromal cells (Kolls et al. (2004) Immunity 21: 467-476; Kawaguchi et al. (2004) J. Allergy Clin. Immunol. 114(6): 1267-1273; Moseley et al. (2003) Cytokine Growth Factor Rev. 14(2): 155-174). Additional IL-17 homologs have been identified (IL-17B, IL-17C, IL-17D, and IL-E) but they share less than 30% amino acid identity with IL-17A (Kolls et al., 2004).

IL-17A is involved in the induction of pro-inflammatory responses and induces or mediates expression of a variety of other cytokines, factors, and mediators including TNF-α, IL-6, IL-8, IL-113, granulocyte colony-stimulating factor (G-CSF), prostaglandin E₂ (PGE₂), IL-10, IL-12, IL-1R antagonist, leukemia inhibitory factor, and stromelysin (Yao et al. (1995) J. Immunol. 155(12): 5483-5486; Fossiez et al. (1996) J. Exp. Med. 183(6): 2593-2603; Jovanovic et al. (1998) J. Immunol. 160: 3513-3521; Teunissen et al. (1998) J. Investig. Dermatol. 111: 645-649; Chabaud et al. (1998) J. Immunol. 161: 409-414). IL-17 also induces nitric oxide in chondrocytes and in human osteoarthritis explants (Shalom-Barak et al., J. Biol. Chem., 273: 27467-27473 (1998); Attur et al., Arthritis Rheum., 40: 1050-1053 (1997)). Through its role in T cell mediated autoimmunity, IL-17 is an important local orchestrator of neutrophil accumulation and plays a role in cartilage and bone destruction of a number of inflammatory diseases.

Although a variety of biologics that specifically bind to IL-17 or TNF-α have been produced since the discovery of these cytokines, there remains a need for improved anti-inflammatory drugs that can effectively mediate or neutralize the activity of both IL-17 and TNF-α in the inflammatory response and autoimmune disorders.

### SUMMARY OF THE INVENTION

The invention provides methods for treating rheumatoid arthritis (RA) in a subject. In various embodiments, the RA is resistant to treatment with one or more disease-modifying antirheumatic drugs (DMARDs). In various embodiments, the subject is about 1-99% resistant to one or more DMARD activities. Such methods comprise administering to a subject (e.g., a human or other mammal) one or more binding proteins that bind IL-17 (e.g., IL-17A) and TNF (e.g., TNF-α). In another embodiment, the invention provides methods for treating RA in a human subject using a binding protein that binds to IL-17 and TNF-α. In certain embodiments, the binding protein is a dual variable domain immunoglobulin (DVD-Ig™) protein. In certain embodiments, administering the binding protein improves a score of one or more RA metrics. In certain embodiments, the binding protein is administered concurrently or subsequently with a DMARD. In certain embodiments, the DMARD comprises methotrexate, sulfasalazine, cyclosporine, leflunomide, hydroxychloroquine, or zathioprine. In various embodiments, the DMARD comprises a chemical e.g., a small molecule or a biologic. For example, the biologic comprises an antibody or ligand receptor. In various embodiments, the binding protein neutralizes TNF and/or IL-17 in vivo. In various embodiments, the binding protein modulates a negative effect of TNF and/or IL-17 in vivo for a period of time. For example, the period of time is at least four hours, 12 hours, one day, three days, a week, two weeks, three weeks, or a month.

In various embodiments, the binding protein comprises three variable heavy (VH) complementarity determining regions (CDRs) for binding TNF-α, wherein the three VH CDRs for binding TNF-α are obtained from a heavy chain variable region comprising the amino acid sequence of SEQ ID NO: 5. In various embodiments, the binding protein comprises three VH CDRs for binding IL-17, wherein the three CDRs are obtained from a heavy chain variable region comprising the amino acid sequence of SEQ ID NO: 7. In various embodiments, the binding protein comprises a heavy chain variable region for binding TNF-α comprising the amino acid sequence of SEQ ID NO: 5. In various embodiments, the binding protein comprises a heavy chain variable region for binding IL-17 comprising the amino acid sequence of SEQ ID NO: 7.

In various embodiments, the binding protein comprises three variable light (VL) CDRs for binding TNF-α, wherein the three VL CDRs are from a light chain variable region comprising the amino acid sequence of SEQ ID NO: 10. In various embodiments, the binding protein comprises three VL CDRs for binding IL-17, wherein the three VL CDRs are from a light chain variable region comprising the amino acid sequence of SEQ ID NO: 12. In various embodiments, the binding protein comprises a light chain variable region for binding TNF-α comprising the amino acid sequence of SEQ ID NO: 10. In various embodiments, the binding protein comprises a light chain variable region for binding IL-17 comprising the amino acid sequence of SEQ ID NO: 12.

In various embodiments, the binding protein comprises the complementarity determining region (CDR) amino acid sequences of the variable heavy chain sequence of SEQ ID NO: 4 or comprises the amino acid sequence of SEQ ID NO: 4. In other embodiments, the binding protein comprises the CDR amino acid sequences of the variable light chain sequence of SEQ ID NO: 9 or comprises the amino acid sequence of SEQ ID NO: 9. In an embodiment, the binding protein comprises the CDR amino acid sequences of the variable heavy chain amino acid sequence of SEQ ID NO: 4 or comprises the amino acid sequence of SEQ ID NO: 4 and comprises the CDR amino acid sequences of the variable light chain amino acid sequence of SEQ ID NO: 9 or comprises the amino acid sequence of SEQ ID NO: 9.

In various embodiments, the binding protein further comprises a constant region. In various embodiments, the constant region comprises at least one mutation compared to a wild-type constant region. In various embodiments, the constant region comprises a variant sequence Fc region. For example, the variant sequence Fc region comprises at least one amino acid mutation. In various embodiments, the constant region comprises the amino acid sequence of SEQ ID NO: 8 or SEQ ID NO: 13.

In various embodiments, the binding protein is administered every day, every two days, every three days, every four days, every five days, every six days, every week, every other week, or every month. In various embodiments, the binding protein is administered at a total dose of between about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg. In a certain embodiment, the binding protein is subcutaneously administered weekly at a dose of about 120 milligrams. In a certain embodiment, the binding protein is subcutaneously administered weekly at a dose of about 200 milligrams or 240 milligrams. In various embodiments, the binding protein is administered at about 200 or 240 mg every other week.

In various embodiments, the binding protein is administered at about 60 mg every other week, about 120 mg every other week, or about 120 mg every other week. In various embodiments, the binding protein is administered at a dose related to the weight of the patient/subject. For example the dose is calculated in milligrams of binding protein per kilogram of patient weight (mg/kg). In various embodiments, the binding protein is administered at a dose of about: 0.1 mg/kg, 0.3 mg/kg, 1.0 mg/kg, 1.5 mg/kg; 2 mg/kg; 3.0 mg/kg, 4 mg/kg; 5 mg/kg; 6 mg/kg; 7 mg/kg; 8 mg/kg; 9 mg/kg; 10 mg/kg; 11 mg/kg; 12 mg/kg; 13 mg/kg; 14 mg/kg; 15 mg/kg; 16 mg/kg; 17 mg/kg; 18 mg/kg; 19 mg/kg; 20 mg/kg; 21 mg/kg; 22 mg/kg; 23 mg/kg; and 24 mg/kg. In various embodiments, the binding protein is formulated for administration to the patient. For example, the binding protein is lyophilized for stability, and then reconstituted with a fluid. For example, the fluid comprises a suspension. In various embodiments, the binding protein is administered using a stock solution at a concentration of about 75, 100, 120, or 150 milligrams per milliliter.

In various embodiments, the binding protein that specifically binds both IL-17 and TNF-α is formulated as a conjugate. In various embodiments, the binding protein is formulated as a pharmaceutical composition comprising a pharmaceutically acceptable carrier. In various embodiments, the method further includes administering to the subject a second agent such as, for example, one or more DMARDs. In certain embodiments, the DMARD is methotrexate.

In various embodiments, administration is systemic or is localized to an area of the subject or diffuses to a treatment area. In various embodiments, the administration is intravenous or by subcutaneous injection.

In various embodiments of the method, the composition is reconstituted from a lyophilized formulation. In various embodiments of the method, the composition comprises at least one substance selected from the group consisting of: sucrose, histidine, polysorbate, and mineral acid. For example, the mineral acid comprises hydrochloric acid. In various embodiments, the method comprises reconstituting the lyophilized composition prior to administering the binding protein.

The binding protein in various embodiments of the method is administered at least once every: day, every other day, every week, every other week, every two weeks, every three weeks, or every month. For example, the binding protein is administered every two weeks. In various embodiments, the amount of binding protein administered over the period of time is constant. In various embodiments, the amount of binding protein administered over the period of time is altered. For example, the amount of binding protein is increased from one administration to the following administration. Alternatively, the amount of binding protein is decreased from one administration to the following administration.

In various embodiments of the method, the subject has been treated with a DMARD or another therapeutic agent (e.g., a steroid, a cyclooxygenase (COX)-2 inhibitor, and acetaminophen) for a period of time prior to administration of the binding protein. In various embodiments, the subject receives a dose of the DMARD of less than 15 mg per week or 10 mg per week. In various embodiments, the subject has been administered a DMARD or other therapeutic agent for a period of time of at least two days, a week, two weeks, three weeks, a month, two months, three months, four months, five months, or six months. In various embodiments, the period of time is about three months. Alternatively, in various embodiments the method comprises administering the DMARD after administering the binding protein.

In various embodiments of the method, administering the binding protein improves at least one negative condition in the subject associated with rheumatoid arthritis. In various embodiments, the negative condition is inflammation, stiffness, pain, bone erosion, osteoporosis, joint deformity, a nerve condition (e.g., tingling, numbness, and burning), scarring, a cardiac disorder, a blood vessel disorder, high blood pressure, fatigue, anemia, weight loss, an abnormal temperature (e.g., fever), a lung disorder, a kidney disorder, a liver disorder, an ocular disorder, a skin disorder, an intestinal disorder, and/or an infection.

In various embodiments of the method, administration of the binding protein to the subject improves a score of one or more rheumatoid arthritis metrics in the subject. For example, the rheumatoid arthritis metric is selected from the group consisting of: Physician Global Assessment of Disease Activity; Patient Reported Outcome; a Health Assessment Questionnaire (HAQ-DI); a patient global assessment of disease activity (VAS)); measurement or presence of an anti-drug antibody (ADA); tender joint count (TJC); swollen joint count (SJC); patient's assessment of pain; Work

Instability Scale for Rheumatoid Arthritis; Short Form Health Survey (SF-36); American College of Rheumatology, ACR, (e.g., ACR20, ACR50, and ACR70); proportion of subjects achieving Low Disease Activity (LDA); Disease Activity Score 28 (DAS28; e.g., DAS28 based on C-reactive protein); Clinical Disease Activity Index (CDAI); simple disease activity index (SDAI); and Clinical Remission criteria.

An aspect of the invention provides methods for treating a subject having rheumatoid arthritis, wherein the subject is resistant to treatment with methotrexate, the method comprising the step of administering to the subject a composition comprising a binding protein that specifically binds both IL-17 and TNF-α, wherein the binding protein is a DVD-Ig™ binding protein, and wherein the binding protein comprises at least one polypeptide comprising an amino acid sequence of SEQ ID NO:4 and an amino acid sequence of SEQ ID NO:9, wherein the binding protein is administered weekly and the total amount administered is about 20 milligrams or about 350 milligrams of the binding protein. For example, for subject is administered about 20-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg of the binding protein. For example, the amount administered is about 60 mg, 120 mg, 180 mg, 200 mg, or 240 mg. In various embodiments, the binding protein is subcutaneously or intravenously administered weekly at a dose of about 120 milligrams. In various embodiments, the binding protein is subcutaneously or intravenously administered weekly at a dose of about 240 milligrams.

An aspect of the invention provides methods of treating a subject having rheumatoid arthritis, wherein the individual has been, or is currently being treated with methotrexate, the method comprising administering to the individual a binding protein that binds both TNF-α and IL-17, wherein the binding protein is a DVD-Ig binding protein, wherein the binding protein comprises a variable heavy chain comprising the amino acid sequence of SEQ ID NO: 4 and comprises a variable light chain comprising the amino acid sequence of SEQ ID NO: 9, wherein administering the binding protein is performed for example using a dose of from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 1.5 mg/kg, from 1.5 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the individual. For example, the binding is administered at a dose of about 1.5 mg/kg. In various embodiments of the method, the binding is administered at a dose of about 3.0 mg/kg. In various embodiments, the binding protein is administered intravenously or subcutaneously. In various embodiments, the binding protein is administered at least once, for example, every day, every other day, every week, every two weeks, every four weeks, and every month. In various embodiments, the binding protein is subcutaneously or intravenously administered.

An aspect of the invention provides methods for treating an individual having rheumatoid arthritis wherein the individual has or is currently being treated with methotrexate, the method comprising: administering to the individual a binding protein that binds both TNF-α and IL-17, wherein the binding protein is a DVD-Ig binding protein, wherein the binding protein comprises a variable heavy chain comprising the amino acid sequence of SEQ ID NO: 4, and comprises a variable light chain comprising the amino acid sequence of SEQ ID NO: 9, wherein administering the binding protein is performed for example using multiple individual doses to reach the total dose. In various embodiments, the total dose is calculated based on a period of time (e.g., days, week, or weeks). For example, the total dose is a weekly total dose and is between about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg of the binding protein. For example the weekly total dose is about 120 mg or 240 mg. In various embodiments, the binding protein is administered at least once, for example every day, every other day, every week, every two weeks, every four weeks, and every month. In various embodiments, the binding protein is subcutaneously or intravenously administered.

An aspect of the invention provides a method for treating a subject having RA, such that the subject is resistant to treatment with methotrexate, the method comprising the step of administering to the subject a composition comprising a binding protein that specifically binds both IL-17 and TNF-α, and the binding protein is a dual variable domain immunoglobulin (DVD-Ig™) protein, and the binding protein comprises at least one polypeptide comprising the amino acid sequence of SEQ ID NO: 4 and the amino acid sequence of SEQ ID NO:9, and the binding protein is administered at from about 60 milligrams to 240 milligrams of the binding protein. For example, the subject is administered 120 milligrams of the binding protein. The binding protein in various embodiments of the method is administered every week. In various embodiments of the method, the binding protein is administered every other week. In various embodiments of the method, the binding protein is administered intravenously. The binding protein in various embodiments of the method is administered subcutaneously. In various embodiments, administering the binding protein is by at least one mode selected from the group consisting of: parenteral, subcutaneous, intramuscular, intravenous, intra-articular, intra-abdominal, intra-capsular, intra-cartilaginous, intra-osteal, intrapelvic, intraperitoneal, intrasynovial, intravesical, bolus, topical, oral, and transdermal.

In various embodiments, the method further comprises administering the composition including the binding protein after administering the methotrexate. Alternatively, the method further comprises administering the composition including the binding protein prior to or currently with administering the methotrexate.

The binding protein in various embodiments of the method is administered at a dosage of about: 0.1 milligram per kilogram of subject weight (mg/kg); 0.3 mg/kg; 1.0 mg/kg; 3 mg/kg; and 10 mg/kg. The composition in various embodiments of the method further comprises at least one substance selected from the group consisting of: sucrose, histidine, polysorbate, and mineral acid.

In various embodiments, the binding protein neutralizes TNF-α and/or IL-17. In various embodiments, the binding protein neutralizes TNF-α and/or IL-17 in vivo for a period of time. In various embodiments, the period of time is at least one selected from the group consisting of: four hours, 12 hours, one day, two days, three days, four days, ten days, 15 days, 18 days, 21 days, 36 days, 48 days, 60 days, 72 days, and 84 days. In various embodiments, the method further comprises observing modulation of a TNF-mediated or an IL-17-mediated symptom or condition.

In various embodiments, the RA affects at least one joint. In various embodiments, the RA is manifested in the subject by the presence of stiffness, pain, swelling, and tenderness of the joints and surrounding ligaments and tendons. In various embodiments, the RA is in a knee, hip, hand, finger, spine/back, toe, and/or foot. In various embodiments, the subject has tendon pain. In various embodiments the subject has at least one joint or nail deformity. In various embodiments, the methods of the invention results in the amelioration of at least one symptom of RA.

In various embodiments, the binding protein comprises a linker comprising the amino acid sequence of SEQ ID NO: 6, SEQ ID NO: 11, or a portion or combination thereof. In various embodiments the linker comprises an amino acid sequence of at least one of SEQ ID NOs: 14-50.

The binding protein in various embodiments comprises a constant region described herein for example in Table 3. For example, the heavy chain constant region may comprise the amino acid sequence of SEQ ID NO: 8 and/or the light chain constant region may comprise the amino acid sequence of SEQ ID NO: 13.

In various embodiments, the binding protein is about 30%, 35%, 40%, 45%, 50%, 55%, 60%, 65%, 70%, 75%, 80%, 85%, 90%, 95%, 96%, 98%, 99%, or 99% or more identical to the amino acid sequence of SEQ ID NO: 4 and/or SEQ ID NO: 9. In a related embodiment, the binding protein comprises a heavy chain variable region that is about 30%, 35%, 40%, 45%, 50%, 55%, 60%, 65%, 70%, 75%, 80%, 85%, 90%, 95%, 96%, 98%, 99%, or 99% or more identical to the amino acid sequence of SEQ ID NO: 4 and/or a light chain variable region that is about 30%, 35%, 40%, 45%, 50%, 55%, 60%, 65%, 70%, 75%, 80%, 85%, 90%, 95%, 96%, 98%, 99%, or 99% or more identical to the amino acid sequence of SEQ ID NO: 9 and retains TNF and IL_{—}17 binding. In a related embodiment, the binding protein comprises 3 CDRs of a heavy chain variable region that are about 30%, 35%, 40%, 45%, 50%, 55%, 60%, 65%, 70%, 75%, 80%, 85%, 90%, 95%, 96%, 98%, 99%, or 99% or more identical to the amino acid sequence of SEQ ID NO: 4 and/or a 3 CDRs of a light chain variable region that are about 30%, 35%, 40%, 45%, 50%, 55%, 60%, 65%, 70%, 75%, 80%, 85%, 90%, 95%, 96%, 98%, 99%, or 99% or more identical to the amino acid sequence of SEQ ID NO: 9.

In various embodiments, the binding protein is formulated in a pharmaceutical composition comprising a pharmaceutically acceptable carrier. In various embodiments, the binding protein is crystallized. In various embodiments, the crystallized binding protein is formulated in a composition comprising an ingredient and/or a polymeric carrier. For example, the polymeric carrier is a polymer selected from the group consisting of poly(acrylic acid), poly(cyanoacrylates), poly(amino acids), poly(anhydrides), poly(depsipeptide), poly(esters), poly(lactic acid), poly(lactic-co-glycolic acid) or PLGA, poly(b-hydroxybutryate), poly(caprolactone), poly(dioxanone); poly(ethylene glycol), poly(hydroxypropyl) methacrylamide, poly [(organo)phosphazene], poly(ortho esters), poly(vinyl alcohol), poly(vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends and copolymers thereof. In various embodiments, the subject is also administered a pain reliever, or a nonsteroidal anti-inflammatory drug (NSAID). Alternatively, the subject is administered a steroid (e.g., a corticosteroid) or a cyclooxygenase (COX)-2 inhibitor.

In various embodiments, the ingredient is selected from one or more of the group consisting of albumin, sucrose, trehalose, lactitol, gelatin, hydroxypropyl-β-cyclodextrin, methoxypolyethylene glycol and polyethylene glycol.

In various embodiments, the binding protein is formulated in a composition or conjugate. For example, the composition or conjugate may comprise sucrose, histidine, and/or a polysorbate (e.g., polysorbate 80). In various embodiments, the binding protein is formulated as a powder and water is added to the composition. In various embodiments, the reconstituted solution comprising the binding protein is administered as an injection. In various embodiments, hydrochloric acid added as necessary to adjust pH. In various embodiments, the binding protein is reconstituted with 1.2 milliliters of sterile water for the injection. In various embodiments, the binding protein being reconstituted is at a concentration of about 100 mg/ml.

In various embodiments, the binding protein is administered at a dosage/dose of about: 0.1 milligram per kilogram of subject weight (mg/kg); 0.3 mg/kg; 1.0 mg/kg; 2 mg/kg; 3 mg/kg; 4 mg/kg; 5 mg/kg; 6 mg/kg; 7 mg/kg; 8 mg/kg; 9 mg/kg, or 10 mg/kg. For example, the dose administered is at least about: from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 1.5 mg/kg; from 1.5 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the individual. In various embodiments, the binding protein is administered at a dose of about 0.1 mg/kg, 0.3 mg/kg, 1.0 mg/kg or 1.5 mg/kg. In various embodiments, the binding protein is administered at a dose of about 3 mg/kg or 10 mg/kg.

The binding protein may be administered using different regimens and administration schedules. For example, the binding protein may be administered once or a plurality of times (e.g., twice, three times, four times to eight times, eight times to ten times, and ten times to twelve times). For example the administration schedule is determined based on the efficacy and/or tolerability of the binding protein in the individual or subject. In various embodiments, the binding protein is administered at least once, for example every day, every other day, every week, every two weeks, every three weeks, every four weeks, and every month. For example the binding protein is administered every week at a dose of about 0.3 mg/kg, 1.0 mg/kg, 1.5 mg/kg, 3 mg/kg, or 10 mg/kg. In various embodiments, the binding protein is administered at a weekly total dose of about 25-375 mg. In an embodiment, the binding protein is subcutaneously administered weekly at a dose of about 240 mg. For example, the binding protein is administered 200-280 mg (e.g., 240 mg) per week.

In various embodiments, the subject has been treated with the DMARD for a period of time prior to administration of the binding protein such that the subject has become resistant to the treatment/therapy. For example, the resistance is least about 1%-10%, 10%-20%, 20%-30%, 30%-40%, 40%-50%, 50%-60%, 60%-70%, 70%-80%, 90-95%, 95%-99%, or 99% resistance to one or more DMARD activities. In various embodiments, the binding protein modulates and reduces the level of resistance by 1%-10%, 10%-20%, 20%-30%, 30%-40%, 40%-50%, 50%-60%, 60%-70%, 70%-80%, 90-95%, 95%-99%, or 99%.

In various embodiments, the method further includes administering the binding protein after administering the DMARD, e.g., methotrexate. Alternatively, the method involves administering the binding protein prior to or concurrently with the DMARD. In a related embodiment of the method, administering the binding protein improves at least one negative condition in the subject associated with RA or RA-associated symptom in the subject. In various embodiments, the at least one RA-associated symptom is selected from the group consisting of one or more of an: autoimmune response (e.g., antibodies and adverse effects), inflammation, stiffness, pain, bone erosion, osteoporosis, joint deformity, joint destruction, a nerve condition (e.g., tingling, numbness, and burning), scarring, a cardiac disorder, a blood vessel disorder, high blood pressure, fatigue, anemia, weight loss, an abnormal temperature (e.g., fever), a lung disorder, a kidney disorder, a liver disorder, an ocular disorder, a skin disorder, an intestinal disorder, and an infection.

In various embodiments, administration of the binding protein to the subject improves a score of one or more RA metrics or criteria in the subject. For example, RA metric and criteria are described in the Examples herein. In various embodiments, the RA metric is selected from the group consisting of one or more of an: American College of Rheumatology Response Rate (ACR for example ACR20, ACR50, and ACR70); proportion of subjects achieving Low Disease Activity (LDA); Disease Activity Score 28 (DAS28; e.g., DAS28 based on C-reactive protein); swollen joints; tender joints patient assessments of pain; global disease activity and physical function; physician global assessment of disease activity and acute phase reactant; disease activity score (DAS) 28; proportion of subjects achieving ACR70 responder status. For example, the binding protein reduces and/or modulates the PsA metric or criteria by at least about 1%, 3%, 5%, 7%, 10%, 15%, 20%, 25%, 30%, 35%, 40%, 45%, 50%, 55%, 60%, 65%, 70%, 75%, 80%, 85%, 90%, 95%, 99% or more.

In various embodiments, the method further comprises observing or detecting a modulation (e.g., reduction or increase) in the presence or activity of a biomarker. In various embodiments, the biomarker is selected from the group consisting of: a high-sensitivity C-reactive protein (hsCRP), a matrix metallopeptidase (MMP; for example MMP-9), a vascular endothelial growth factor (VEGF), an MMP degradation product for example an MMP degradation product of type I, II, or III collagen (C1M, C2M, C3M), a C-reactive protein (CRPM), a prostaglandin, nitric oxide, a disintegrin and metalloproteinase with thrombospondin motifs (ADAMTS), an adipokine, an endothelial growth factor (EGF), a bone morphogenetic protein (BMP), a nerve growth factor (NGF), a substance P, an inducible Nitric Oxide Synthase (iNOS), CTX-I, CTX-II, TIINE, creatinine, and a vimentin (for example a citrullinated and MMP-degraded vimentin; VICM). In various embodiments of the method, the binding protein reduces the severity of the RA and/or modulates (e.g., reduces and increases) expression and/or activity of the biomarker by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments, the method further comprises administering the composition including the binding protein after having administered the methotrexate. In various embodiments, the method further comprises administering another agent to the subject. For example, the additional agent is selected from the group consisting of: a therapeutic agent, an imaging agent, a cytotoxic agent, an angiogenesis inhibitor, a kinase inhibitor, a co-stimulation molecule blocker, an adhesion molecule blocker, an anti-cytokine antibody or functional fragment thereof, methotrexate, cyclosporine, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteroid, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog thereof, a cytokine, and a cytokine antagonist. Alternatively, the binding protein is administered concurrently or prior to administering the methotrexate.

In various embodiments the method comprises identifying an improvement in the subject in the severity or duration of a symptom associated with rheumaotid arthritis. For example, identifying an improvement comprises using a score, a test, or a metric for RA or inflammation. In various embodiments of the method, the score, the test, or the metric is selected from the group consisting of one or more of American College of Rheumatology Response Rate (ACR for example ACR20, ACR50, and ACR70); proportion of subjects achieving Low Disease Activity (LDA); Disease Activity Score 28 (DAS28; e.g., based on C-reactive protein); swollen joints; tender joints patient assessments of pain; global disease activity and physical function; physician global assessment of disease activity and acute phase reactant levels; and proportion of subjects achieving ACR70 responder status.

An aspect of the invention provides a method for treating rheumatoid arthritis in a human subject comprising the step of administering to the human subject a binding protein that specifically binds both TNF-α and IL-17, wherein the binding protein is a DVD-Ig binding protein including a variable heavy chain comprising an amino acid sequence of SEQ ID NO: 4, and including a variable light chain comprising an amino acid sequence of SEQ ID NO: 9, in a dose to achieve:

(a) an area under the curve (AUC) of between about 1 and about 500 μg·day/mL;

(b) a serum or plasma half-life (T_{1/2}) of at least about 2 to 40 days, e.g., about 33 days;

(c) a time point to maximum observed serum concentration (Tmax) of between about 1 days and about 10 days;

(d) a maximum observed serum concentration (Cmax) of between about 0.5 and about 400 g/mL;

(e) an improvement of a negative condition or symptom associated with rheumatoid arthritis; and/or

(f) an improvement a score or criteria of one or more rheumatoid arthritis metric.

In various embodiments, AUC is between about 17 and about 448 μg-day/mL.

In various embodiments, the T_{1/2} is at least about 5 and about 11 days. In various embodiments, the Tmax is between about 1 and 7 days. In various embodiments, the Cmax is between about 2 and about 81 μg/mL. In various embodiments, the value or range for the AUC, T_{1/2}, Tmax, or Cmax is described herein.

In various embodiments of the method, the negative condition or symptom is selected from the group consisting of: autoimmune response; inflammation; stiffness; pain; bone erosion/osteoporosis; joint deformity; joint destruction, a nerve condition; scarring; a cardiac disorder/condition; a blood vessel disorder/condition; high blood pressure; tiredness; anemia; weight loss; an abnormal temperature; a lung condition/disease; a kidney condition/disorder; a liver condition/disorder; an ocular disorder/condition; a skin disorder/condition; an intestinal disorder/condition; and an infection. In an embodiment, the binding protein reduces the negative symptom by about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments, the rheumatoid arthritis metric is selected from the group consisting of: Physician Global Assessment of Disease Activity; Patient Reported Outcome; a HAQ-DI; a patient global assessment of disease activity (VAS); measurement or presence of an ADA; TJC; SJC; patient's assessment of pain; Work Instability Scale for Rheumatoid Arthritis; Short Form Health Survey; American College of Rheumatology, ACR, (e.g., ACR20, ACR50, and ACR70); proportion of subjects achieving LDA; Disease Activity Score 28; DAS28 based on C-reactive protein; CDAI; SDAI; and Clinical Remission criteria.

In an embodiment, the binding protein reduces the metric by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

The subject in various embodiments of the method is resistant to treatment with at least one disease-modifying antirheumatic drug (DMARD). For example, the DMARD is selected from the group consisting of methotrexate, sulfasalazine, cyclosporine, leflunomide, hydroxychloroquine, and zathioprine.

In various embodiments of the method, administering the binding protein is by at least one mode selected from the group consisting of: parenteral, subcutaneous, intramuscular, intravenous, intra-articular, intra-abdominal, intra-capsular, intra-cartilaginous, intra-osteal, intrapelvic, intraperitoneal, intrasynovial, intravesical, bolus, topical, oral, and transdermal. In various embodiments, the binding protein is administered every day, every two days, twice per week, once per week, every two weeks, every other week, every three weeks, every month, every two months, or every few months. In various embodiments, the binding protein is administered in a single dose. In various embodiments, the binding protein is administered in multiple doses.

In various embodiments, the method further comprises administering another therapeutic agent. In various embodiments of the method, the therapeutic agent comprises a DMARD.

In various embodiments, the binding protein is administered at a dosage from the group consisting of: 0.1 milligram per kilogram of subject weight (mg/kg); 0.3 mg/kg; 1.0 mg/kg; 1.5 mg/kg; 2 mg/kg; 3 mg/kg; 4 mg/kg; 5 mg/kg; 6 mg/kg; 7 mg/kg; 8 mg/kg; 9 mg/kg; 10 mg/kg; 11 mg/kg; 12 mg/kg; 13 mg/kg; 14 mg/kg; 15 mg/kg; 16 mg/kg; 17 mg/kg; 18 mg/kg; 19 mg/kg; 20 mg/kg; 21 mg/kg; 22 mg/kg; 23 mg/kg; and 24 mg/kg. For example, the binding protein is administered at a dose selected from the group consisting of: from about 0.5 mg/kg, 1 mg/kg, 1.5 mg/kg, and 3 mg/kg.

In various embodiments, the binding protein is administered at a dose from the group consisting of about: 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, about 325-350 mg, 350-375 mg, or 375-400 mg of the binding protein. For example, the dose comprises a dose described herein. In various embodiments, the dose is at least about from 60 mg, 120 mg, 200 mg, or 240 mg. In various embodiments, the dose is administered weekly or every other week.

### BRIEF DESCRIPTION OF THE DRAWINGS

FIG. 1 panel A is a graph showing serum concentration (μg/mL; ordinate) of ABT-122 in cynomolgus monkeys as a function of time (days; abscissa). Each monkey (N=3) was intravenously injected with a single dose (5 mg/kg) of ABT-122.

FIG. 1, panel B is a graph showing measured (circle) and simulated (line) serum concentration (μg/mL; ordinate) of ABT-122 in a representative cynomolgus monkey as a function of time (days; abscissa). The subject (N=1) received an intravenously injected every week for four weeks (Q1Wx4) a single dose (45 mg/kg) of ABT-122.

FIG. 2, panel A and FIG. 2, panel B are a set of graphs showing ABT-122 serum concentrations (ordinate; μg/mL) as a product of time (abscissa; days) after an intravenous single dose administration or after subcutaneous single dose administration, respectively (Study M12-704). Serum concentrations are shown in a log-linear scale (mean+SD). FIG. 2 panel A shows ABT-122 serum concentrations in human subjects after intravenous administration of different doses of ABT-122: 0.1 mg/kg (circle); 0.3 mg/kg (downward triangle); 1.0 mg/kg (square); 3.0 mg/kg (diamond), and 10 mg/kg (upward facing triangle). FIG. 2 panel B shows ABT-122 serum concentrations in human subjects after subcutaneous administration of different doses of ABT-122: 0.3 mg/kg (downward triangle); 1.0 mg/kg (square); and 3.0 mg/kg (diamond).

FIG. 3 is a graph showing data for a TNF/IL-17 human fibroblast-like synoviocyte (FLS) potency assay. ABT-122 was serially diluted and placed in human serum and was incubated for 1 hour with a mixture of TNF-α (0.2 ng/mL) and IL-17 (2 ng/mL). The ABT-122/TNF-α/I1-17 solution was then contacted with human FLS. Control samples without ABT-122 were also prepared that were without TNF and IL-17, with TNF only, or with IL-17 only. Samples were incubated overnight and supernatants were collected. The graph shows the concentration of IL-6 (ordinate; pg/mL) in FLS supernatants) as a function of contacted ABT-122 (abscissa; nM).

FIG. 4 shows a TNF/IL-17 human FLS potency assay using serum from subjects treated with ABT-122. Subjects were intravenously injected with 1 mg/kg of ABT-122 or were subcutaneously administered ABT-122. Human sera from subjects treated with ABT-122 were diluted (1:20 to 1:250 for IV treatment and 1:10 to 1:20 for subcutaneous treatment) and was incubated for 1 hour with a mixture of TNF-α (0.2 ng/mL) and IL-17 (2 ng/mL). The ABT-122/TNF-α/I1-17 solution was then contacted with human FLS. Control samples without ABT-122 were also prepared that were without TNF and IL-17, with TNF only, or with IL-17 only. Samples were incubated overnight and supernatants were collected. The graph showing the concentration of IL-6 (ordinate; pg/mL) in FLS supernatants as a function of ABT-122 serum dilution contacted to the sample (abscissa). Dilutions were different for each cohort to fit curves in range on assay.

FIG. 5 panel A, FIG. 5 panel B, FIG. 5 panel C, and FIG. 5 panel D, are a set of graphs showing TNF-α and IL-17 neutralization ability of serum from subjects subcutaneously injected with a control that did not contain ABT-122 (placebo), or with 0.1 mg/kg, 1 mg/kg, or 3 mg/kg of ABT-122, respectively.

FIG. 6, panel A is a graph of the IC50 (ordinate; ng/mL) in vitro analysis for subjects administered with ABT-122.

FIG. 6, panel B is a graph of the IC90 (ordinate; ng/mL) in vitro analysis for subjects administered with ABT-122.

FIG. 7, panel A is a graph showing percentage of TNF and IL-17 stimulation (ordinate) as a function of dilution of serum from healthy subjects four hours after being intravenously administered different concentrations of ABT-122. The subjects were administered ABT-122 at a concentration of: 0.1 mg/kg (filled circle); 0.3 mg/kg (square); 1.0 mg/kg (diamond); and 10 mg/kg (triangle). FIG. 7, panel B is a graph showing IL-6 concentration (pg/mL; ordinate) as a function of degree of dilution of serum from samples from healthy subjects a period of time after being intravenously administered 1 mg/kg of ABT-122. The serum dilutions were obtained from samples obtained after 0 hours, 4 hours, 3 days, 10 days, 21 days and 84 days.

FIG. 8 is a graph showing ABT-122 serum concentration (μg/mL; ordinate) for as a function of time for rheumatoid arthritis subjects treated with methotrexate and ABT-122 in studies M12-962 and M14-048. FIG. 8 shows PK data for subjects administered 0.5 mg/kg EW to 3 mg/kg EW of ABT-122 for 8 weeks.

FIG. 9 shows the change in baseline hsCRP, neutrophil and DAS28 CRP values from studies M12-962 and M14-04. The change from baseline was measured to Day 57 in hsCRP and DAS28 (CRP) and to Day 8 in neutrophils. No minimum disease activity was specified as entry criteria for the MAD studies. Pooled data M12-962 and M14-048 Change from baseline was measured to Day 57 in hsCRP and DAS28(CRP) and to Day 8 in Neutrophils. Day 57 one week after last day of dosing Placebo patients was pooled from across arms (six active: 2 placebo) at five sites.

FIG. 10, panel A is a graph showing serum concentration (μm/ml; ordinate) of ABBV-257 as a function of time (abscissa; hours) for CD-1 mice intravenously administered the DVD-Ig binding protein (5 mg/kg). Animals with apparent ADA were excluded from pharmacokinetic calculations.

FIG. 10, panel B is a graph showing serum concentration (μm/ml; ordinate) of ABBV-257 as a function of time (abscissa; hours) for Sprague Dawley rats intravenously administered the DVD-Ig binding protein (5 mg/kg).

FIG. 11 is a graph showing serum concentration values (μm/ml; ordinate) as function of time (abscissa; hours) for female cynomolgus monkeys following weekly 100 mg/kg intravenous doses of ABBV-257.

FIG. 12, panel A is a graph showing serum concentrations of ABBV-257 (m/mL; ordinate) as a function of time (abscissa; hours) for cynomolgus monkeys administered weekly intravenous doses (60 or 200 mg/kg) of the binding protein or administered weekly subcutaneous doses (200 mg/kg) of the binding protein.

FIG. 12, panel B is a graph showing trough concentrations of ABBV-257 (m/mL; ordinate) as a function of time (abscissa; days) in cynomolgus monkeys administered weekly intravenous doses (60 mg/kg, square; or 200 mg/kg, circle) of the binding protein or administered weekly subcutaneous doses (200 mg/kg, triangle) of the binding protein.

For FIG. 12, panel A and FIG. 12 panel, B the values are shown as mean (±SD). N=4 to 8 and samples were obtained after dosing on D1 (Day 1), D22 (Day 22), and D50 (Day 50).

FIG. 13, panel A, is a graph showing serum concentrations of ABBV-257 (m/mL; ordinate) as a function of time (abscissa; days) for human patients intravenously administered a single dose (0.3 mg/kg, 1.0 mg/kg, or 3.0 mg/kg) of ABBV-257. N=6 per dose group; intravenous administration was a continuous infusion over six hours.

FIG. 13, panel B, is a graph showing serum concentrations of ABBV-257 (m/mL; ordinate) as a function of time (abscissa; days) for human patients subcutaneously administered a single dose (0.3 mg/kg or 3.0 mg/kg) of ABBV-257. N=6 per dose group.

### DETAILED DESCRIPTION OF THE INVENTION

Rheumatoid arthritis is an autoimmune disease that produces a number of effects in subjects, including inflammation, warmth, redness, swelling, and pain. During the inflammation process, the normally thin synovium becomes thick and makes the joint swollen, puffy, and sometimes warm to the touch. As rheumatoid arthritis progresses, the inflamed synovium invades and destroys the cartilage and bone within the joint. The surrounding muscles, ligaments, and tendons that support and stabilize the joint become weak and unable to work normally. These effects lead to the pain and joint damage often seen in rheumatoid arthritis. Wolfe et al. (2007) Arthrit. Rheum. 56(7):2135-2142. DMARDs are used to treat these effects; however it is often observed that over time the patients fail to effectively respond to the DMARDs.

This invention pertains to methods of using binding proteins, or antigen-binding portions thereof, that bind IL-17 or TNF-α, such as DVD-Ig™ binding proteins that bind IL-17 and TNF-α to treat rheumatoid arthritis (RA), RA-associated symptoms and/or DMARD-resistant RA. Bi-specific antibodies and antibody fragments thereof, DVD-Ig™| binding proteins, and pharmaceutical compositions thereof, as well as nucleic acids, recombinant expression vectors and host cells for making such IL-17/TNF binding proteins are used to treat the RA. Methods of using the IL-17/TNF binding proteins to detect human IL-17A homodimer and/or IL-17A/F heterodimer, either in vitro or in vivo, and to regulate gene expression are also encompassed by the invention. The invention also encompasses use of any binding protein or antibody capable of competing with an IL-17/TNF-α binding protein described herein. In certain embodiments, the binding protein is a DVD-Ig™ protein comprising one or more of the sequences shown in Example 1.

ABT-122 is an IgG1 dual-variable domain immunoglobulin (DVD-Ig™) binding protein described herein that specifically binds and neutralizes the pro-inflammatory cytokines tumor necrosis factor (TNF, also known as TNF-α) and Interleukin 17 (IL-17) and prevents them from binding to their respective receptors on cells. TNF is typically a soluble homotrimer, after being enzymatically cleaved from the cell surface (Tracey et al. (2008) Pharmacol Ther. 117 (2):244-79). ABT-122, binds to the IL-17A component, thereby neutralizing IL-17A homodimers and IL-17A-F heterodimers but not to other members of the IL-17 family. The ABT-122 molecule has 2 sets of variable domain sequences connected in tandem by flexible peptide linkers, and has preserved human immunoglobulin G1 (IgG1) heavy chain and κ light chain constant regions. Human IgG1 molecules found in nature are bivalent and monospecific with a molecular weight of approximately 150 kilodaltons. In ABT-122, the heavy and light chains form a tetravalent, bi-specific immunoglobulin-like molecule with a molecular weight of 198 kilodaltons.

TNF and IL-17 have important roles in the pathogenesis of RA and other inflammatory diseases. Both cytokines are expressed at increased levels in synovial tissue and are key factors in the joint inflammation and damage to bone and cartilage that are hallmarks of the disease (Frleta et al. (2014) Curr. Rheumatol. Rep. 16(4):414). TNF blockade is an established therapy for RA. IL-17 blockade has demonstrated efficacy in psoriasis (Langley et al. (2014) N. Engl. J. Med. 371(4):326-38; Papp et al. (2012) N. Engl. J. Med. 366(13):1181-9; Tham et al. (2014) J. Clin. Pharmacol. 54(10):1117-24). Trials are currently being conducted in RA and other inflammatory diseases (Gisondi et al. (2014) Dermatol. Ther. (Heidelb) 4(1):1-9; McInnes et al. (2014) Ann. Rheum. Dis. 73(2):349-56; Mease et al. (2014) N. Engl. J. Med. 370(24):2295-306). Without being limited by any particular theory or mechanism of action, it is here envisioned that ABT-122 binding protein as described herein is as effective or more effective than current treatments for rheumatoid arthritis.

In the first-in-human, single ascending dose study of ABT-122 (Study M12-704), 48 healthy volunteers were administered a single dose of ABT-122, ranging from 0.1 mg/kg to 10 mg/kg by intravenous (IV) administration and 0.3 mg/kg to 3 mg/kg by subcutaneous (SC) administration. No events of severe intensity, serious adverse events, systemic hypersensitivity reactions or injection site reactions, discontinuations due to adverse events, or deaths occurred. There were no dose limiting toxicities and no apparent association of particular adverse events with dose or route of administration. Following SC administration, ABT-122 absolute bioavailability was ˜50% and the maximum serum concentrations were observed 3 to 4 days after dosing. The majority of subjects had detectable anti-drug antibodies (ADA) across all dose groups, although they were largely exhibiting low titer values.

The presence of ADA did not appear to influence drug clearance for majority of the subjects and did not correlate with any systemic or serious adverse event profiles.

The present invention provides methods for treating rheumatoid arthritis (RA) in a subject. Generally, the subject is a human. For example, in various embodiments the subject has RA and is resistant to treatment with one or more disease-modifying antirheumatic drugs (DMARDs). Such methods which are an aspect of the invention comprise administering to a subject (e.g., human or other mammal) one or more binding proteins that bind both IL-17 and TNF (e.g., TNF-α). In another embodiment, the invention provides methods for treating RA in a human subject using a binding protein that binds and/or neutralizes both IL-17 and TNF-α. In certain embodiments, the binding protein is a dual variable domain immunoglobulin (DVD-Ig™) protein. In certain embodiments, administering the binding protein improves a score of one or more RA metrics or criteria. In various embodiments of the method, the DMARD comprises methotrexate. In various embodiments, the binding protein neutralizes TNF and/or IL-17 in vivo. In various embodiments, the binding protein modulates one or more negative effects of TNF and/or IL-17 in vivo for a period of time after administration of a dose. For example, the period of time is at least four hours, 12 hours, one day, three days, a week, two weeks, three weeks, or a month.

In various embodiments, the binding protein comprises the CDR amino acid sequences of the variable heavy chain sequence of SEQ ID NO: 4, or comprises the amino acid sequence of SEQ ID NO: 4. In other embodiments, the binding protein comprises the CDR amino acid sequences of the variable light chain sequence of SEQ ID NO: 9, or comprises the amino acid sequence of SEQ ID NO: 9. In an embodiment, the binding protein comprises the CDR amino acid sequences of the variable heavy chain amino acid sequence of SEQ ID NO: 4, or comprises the amino acid sequence of SEQ ID NO: 4 and comprises the CDR amino acid sequences of the variable light chain amino acid sequence of SEQ ID NO: 9, or comprises the amino acid sequence of SEQ ID NO: 9. In various embodiments, the binding protein is administered every day, every few days, every week, every other week, or every month.

In a related embodiment, the binding protein comprises the heavy chain constant region comprising the amino acid sequence of SEQ ID NO: 8. In a related embodiment of the method, the binding protein comprises the light chain constant region comprises the amino acid sequence of SEQ ID NO: 13.

In various embodiments, the binding protein is administered at about 60 mg every other week, about 120 mg per week, or about 120 mg every other week. In various embodiments, the binding protein is administered every week for example about 50-100 mg, 100-150 mg, 150-200 mg, 200-250 mg, 250-300 mg, or 300-350 mg. For example, the binding protein is administered at a dose of 240 mg per week. In various embodiments, the binding protein is administered at a dose related to the weight of the patient/subject. For example the dose is calculated in milligrams of binding protein per kilogram of patient weight (mg/kg). In various embodiments, the binding protein is formulated for administration to the patient. For example, the binding protein is lyophilized for stability, and then reconstituted with a fluid.

The methods of the invention may include the use of a “therapeutically effective amount” of the TNFα/IL-17 DVD-Ig binding protein. A “therapeutically effective amount” refers to an amount effective, at dosages and for periods of time necessary, to achieve the desired therapeutic result. A therapeutically effective amount of the TNFα/IL-17 DVD-Ig binding protein may be determined by a person skilled in the art and may vary according to factors such as the disease state, age, sex, and weight of the individual, pharmacokinetics, pharmacogenetics, bioavailability, and the ability of the TNFα/IL-17 DVD-Ig binding protein to elicit a desired response in the individual. A therapeutically effective amount is also one in which any toxic or detrimental effects of the TNFα/IL-17 DVD-Ig binding protein are outweighed by the therapeutically beneficial effects.

Administering the binding protein is performed in various embodiments using a dose of at least: from 0.005 (milligrams per kilogram) mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, from 9 mg/kg to 10 mg/kg, from 10 mg/kg to 11 mg/kg, from 11 mg/kg to 12 mg/kg, from 12 mg/kg to 13 mg/kg, from 13 mg/kg to 14 mg/kg, from 14 mg/kg to 15 mg/kg, from 15 mg/kg to 16 mg/kg, from 16 mg/kg to 17 mg/kg, from 17 mg/kg to 18 mg/kg, from 18 mg/kg to 19 mg/kg, from 19 mg/kg to 20 mg/kg, from 20 mg/kg to 21 mg/kg, from 21 mg/kg to 22 mg/kg, from 22 mg/kg to 23 mg/kg, from 23 mg/kg to 24 mg/kg, from 24 mg/kg to 25 mg/kg, from 25 mg/kg to 26 mg/kg, from 26 mg/kg to 27 mg/kg, from 27 mg/kg to 28 mg/kg, from 28 mg/kg to 29 mg/kg, from 29 mg/kg to 30 mg/kg, or from 30 mg/kg to 40 mg/kg of weight of the binding protein to weight of the individual. In various embodiments, the binding protein is administered at 0.1 mg/kg, 0.3 mg/kg, 1 mg/kg, 3 mg/kg, or 10 mg/kg.

In certain embodiments, the binding protein is administered as a total dose at a particular point in time of between about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, about 325-350 mg, about 350 mg-400 mg of the binding protein. In certain embodiments, a total dose of between about 25 mg and about 400 mg is administered. In various embodiments, the binding protein is administered at a dose of about 60 mg or about 120 mg.

Dosage regimens may be adjusted to provide the optimum desired response (i.e., a therapeutic response). For example, a single dose (e.g., bolus) may be administered, several divided doses may be administered over time or the dose may be proportionally reduced or increased as indicated by the exigencies of the therapeutic situation. In certain embodiments, an initial dose is administered, followed by the administration of one or more subsequent doses at a later date in time. For example, an initial dose may be administered to a subject on day 1, followed by one or more subsequent doses, e.g., every week, twice a week, every two weeks, every three weeks, every four weeks, etc. for a given period of time.

The parenteral compositions may be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. Dosage unit forms are dictated by (a) the unique characteristics of the active compound and the particular therapeutic or prophylactic effect to be achieved, and (b) the limitations inherent in the art of compounding such an active compound for the treatment an individual. Dosage values may vary with the type and severity of the condition to be alleviated. Specific dosage regimens should be adjusted over time according to individual need. Dosage ranges set forth herein are exemplary only and are not intended to limit the scope or practice of the claimed composition.

The TNFα/IL-17 DVD-Ig binding protein can be incorporated into pharmaceutical compositions suitable for administration to a subject. In various embodiments, the pharmaceutical composition comprises a TNFα/IL-17 DVD-Ig binding protein and a pharmaceutically acceptable carrier. The term “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of substances such as wetting or emulsifying agents, preservatives or buffers, which enhance the shelf life, stability, or effectiveness of the pharmaceutical composition.

Various delivery systems are known and can be used to administer the TNFα/IL-17 DVD-Ig binding protein for preventing or treating RA or one or more symptoms thereof, e.g., encapsulation in liposomes, microparticles, microcapsules, recombinant cells capable of expressing the antibody or antibody fragment, receptor-mediated endocytosis (see, e.g., Wu and Wu (1987) J. Biol. Chem. 262: 4429-4432), construction of a nucleic acid as part of a retroviral or other vector. Methods of administering the TNFα/IL-17 DVD-Ig binding protein include, but are not limited to, parenteral administration (e.g., intradermal, intramuscular, intraperitoneal, intravenous and subcutaneous), epidural administration, intratumoral administration, transdermal (e.g., topical), rectal and transmucosal administration (e.g., intranasal and oral routes)). In addition, pulmonary administration can be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244, WO 97/32572, WO 97/44013, WO 98/31346, and WO 99/66903, each of which is incorporated herein by reference their entireties. In one embodiment, the TNFα/IL-17 DVD-Ig binding protein is administered using Alkermes AIR® pulmonary drug delivery technology (Alkermes, Inc., Cambridge, Mass., US). In a specific embodiment, the TNFα/IL-17 DVD-Ig binding protein is administered intramuscularly, intravenously, intratumorally, orally, intranasally, pulmonary, or subcutaneously. The TNFα/IL-17 DVD-Ig binding protein may be administered by any convenient route, for example by infusion or bolus injection, by absorption through epithelial or mucocutaneous linings (e.g., oral mucosa, rectal, and intestinal mucosa, etc.) and may be administered together with other biologically active agents. Administration can be systemic or local. Local administration may be by local infusion, injection, or by means of an implant, e.g., of a porous or non-porous material, including membranes and matrices, such as sialastic membranes, polymers, fibrous matrices (e.g., Tissuel®), or collagen matrices. In various embodiments, an effective amount of the TNFα/IL-17 DVD-Ig binding protein is administered locally to the affected area of a subject to prevent or treat RA or a symptom thereof. In various embodiments, an effective amount of the TNFα/IL-17 DVD-Ig binding protein is administered locally to the affected area in combination with an effective amount of one or more therapies (e.g., one or more prophylactic or therapeutic agents) other than the TNFα/IL-17 DVD-Ig binding protein to prevent or treat RA or one or more symptoms thereof.

In another embodiment, the TNFα/IL-17 DVD-Ig binding protein can be delivered in a controlled release or sustained release system, e.g., via a pump (see Langer, supra; Sefton (1987) CRC Crit. Ref. Biomed. Eng. 14:20; Buchwald et al. (1980) Surgery 88:507; Saudek et al. (1989) N. Engl. J. Med. 321:574). In another embodiment, polymeric materials can be used to achieve controlled or sustained release of the TNFα/IL-17 DVD-Ig binding protein (see, e.g., Goodson Chapter 6, In Medical Applications of Controlled Release, Vol. II, Applications and Evaluation, (Langer and Wise, eds.) (CRC Press, Inc., Boca Raton, 1984), pp. 115-138; Ranger and Peppas (1983) J. Macromol. Sci. Rev. Macromol. Chem. 23:61; Levy et al. (1985) Science 228:190; During et al. (1989) Ann. Neurol. 25:351; Howard et al. (1989) J. Neurosurg. 7 1:105); U.S. Pat. Nos. 5,679,377; 5,916,597; 5,912,015; 5,989,463; and 5,128,326; and PCT Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of use in the practice of the invention. See, e.g., U.S. Pat. No. 4,526,938; PCT publication WO 91/05548; PCT publication WO 96/20698; Ning et al. (1996) Radiother. Oncol. 39: 179-189; Song et al. (1995) PDA J. Pharm. Sci. Tech. 50: 372-397; Cleek et al. (1997) Pro. Intl. Symp. Control. Rel. Bioact. Mater. 24: 853-854; and Lam et al. (1997) Proc. Intl. Symp. Control Rel. Bioact. Mater. 24: 759-760; each of which is incorporated herein by reference in its entirety.

In an embodiment, where the composition is a nucleic acid encoding the TNFα/IL-17 DVD-Ig binding protein, the nucleic acid can be administered in vivo to promote expression of the TNFα/IL-17 DVD-Ig binding protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al. (1991) Proc. Natl. Acad. Sci. USA 88: 1864-1868). Alternatively, a nucleic acid can be introduced intracellularly and incorporated within host cell DNA for expression, e.g., by homologous recombination.

The TNFα/IL-17 DVD-Ig binding protein pharmaceutical composition used in the methods of the invention is formulated to be compatible with its intended route of administration. Typically, compositions for intravenous administration are solutions in sterile isotonic aqueous buffer. The composition may also include a solubilizing agent or a local anesthetic, such as lignocaine, to ease pain at the site of the injection.

If the TNFα/IL-17 DVD-Ig binding protein compositions are administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as FREON®) or in a squeeze bottle. Moisturizers or humectants can also be added to pharmaceutical compositions and dosage forms if desired. Examples of such additional ingredients are well known in the art.

If the methods of the invention comprise intranasal, the TNFα/IL-17 DVD-Ig binding protein composition can be formulated in the form of an aerosol, spray, mist or drops. The TNFα/IL-17 DVD-Ig binding protein can be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide). In the case of a pressurized aerosol, the dosage unit may be determined by providing a valve to deliver a metered amount. Capsules and cartridges (e.g., composed of gelatin) for use in an inhaler or insufflator may be formulated containing a powder mix of the compound and a suitable powder base such as lactose or starch.

If the methods of the invention comprise oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The methods of the invention may comprise pulmonary administration, e.g., by use of an inhaler or nebulizer, of a composition formulated with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244, WO 97/32572, WO 97/44013, WO 98/31346, and WO 99/66903, each of which is incorporated herein by reference their entireties.

The methods of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the invention may encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The ingredients of compositions may be supplied either separately or mixed together in unit dosage form, for example, as a dry lyophilized powder or water free concentrate in a hermetically sealed container such as an ampoule or sachet indicating the quantity of active agent. In various embodiments where the mode of administration is infusion, composition can be dispensed with an infusion bottle containing sterile pharmaceutical grade water or saline. In various embodiments where the mode of administration is by injection, an ampoule of sterile water for injection or saline can be provided so that the ingredients may be mixed prior to administration.

In particular, the methods of the invention also provide that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of use in the practice of the invention is supplied as a dry sterilized lyophilized powder or water free concentrate in a hermetically sealed container and can be reconstituted (e.g., with water or saline) to the appropriate concentration for administration to a subject.

The TNFα/IL-17 DVD-Ig binding protein may be a pharmaceutical composition suitable for parenteral administration, e.g., an injectable solution. The injectable solution can be composed of either a liquid or lyophilized dosage form in a flint or amber vial, ampoule or pre-filled syringe (see, WO 2004/078140 and U.S. Patent Publication No. 2006104968).

The compositions may be in a variety of forms, including for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application.

Therapeutic compositions typically are sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., binding protein) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

In certain embodiments, the active compound may be prepared with a carrier that protects the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The TNFα/IL-17 DVD-Ig binding protein may be orally administered, for example, with an inert diluent or an edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound according to the methods of the invention by other than parenteral administration, it may be necessary to coat the compound with, or co-administer the compound with, a material to prevent its inactivation.

In certain embodiments the TNFα/IL-17 DVD-Ig binding protein is linked to a half-life extending vehicle known in the art. Such vehicles include, but are not limited to, the Fc domain, polyethylene glycol, and dextran. Such vehicles are described, e.g., in U.S. Pat. No. 6,660,843 and published PCT Publication No. WO 99/25044, which are hereby incorporated by reference for any purpose.

In various embodiments, the step of administering to an individual is by at least one mode of administration selected from: parenteral, subcutaneous, intramuscular, intravenous, intraarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, topical, oral, and transdermal. For example, the binding protein is subcutaneously administered as described in any of the working examples herein. Alternatively, the binding protein is intravenously administered as described in any of the working examples herein.

Administering the binding protein is performed in various embodiments at least two times or is performed periodically. For example the binding protein is administered at least two times, at least three times, or at least four times over a period of time. In various embodiments, the binding protein is administered multiple times to the individual over a period of days, weeks, months or years.

In various embodiments, the binding protein that specifically binds both IL-17 and TNF-α is formulated in a pharmaceutical composition comprising a pharmaceutically acceptable carrier. In various embodiments, the method further includes administering to the subject a second agent such as, for example, one or more DMARDs. In certain embodiments, the DMARD is methotrexate. In various embodiments, the DMARD is synthetic. In various embodiments, the DMARD is or comprises a biologic. In various embodiments, the DMARD is or comprises a small molecule. In various embodiments the DMARD is a sulfasalazine, an auranofin, a gold compound, an azathioprine, a 6-mercaptopurine, a ciclosporin A, an antimalarial agent, d-penicillamine, or a retinoid or combination thereof.

In various embodiments, administration is systemic or is localized to an area of the subject or diffuses to a treatment area. In various embodiments, the administration is intravenous or by subcutaneous injection.

In various embodiments, the composition is lyophilized. In various embodiments of the method, the composition comprises at least one substance selected from the group consisting of:

sucrose, histidine, polysorbate, and mineral acid. For example, the mineral acid comprises hydrochloric acid.

The composition in various embodiments of the method comprises the binding protein at a concentration of about 100 milligrams per milliliter. The binding protein in various embodiments of the method is administered at least once every: day, every other day, every week, every two weeks, and every month. For example, the binding protein is administered every two weeks.

The subject in various embodiments of the method has been treated with the DMARD for a period of time prior to administration of the binding protein. For example, the period of time is at least two days, a week or a month. In various embodiments, the period of time is about three months.

The subject in various embodiments of the methods of the invention has been receiving treatment with the DMARD for a period of time, e.g., a few days, weeks or months. The method in various embodiments further includes administering the binding protein after administering the DMARD, e.g. administered minutes, hours, days or months afterward. The method in various embodiments further includes administering the binding protein concomitant with administering the DMARD. Alternatively, the binding protein is administered minutes, hours, days or months prior to administering the DMARD.

In various embodiments of the method, administering the binding protein improves at least one a negative condition in the subject associated with the RA, or RA associated symptom. In various embodiments, the RA associated symptom is selected from the group consisting of inflammation; stiffness; pain; bone erosion/osteoporosis; joint deformity; a nerve condition (e.g., tingling, numbness, and burning); scarring; a cardiac disorder/condition; a blood vessel disorder/condition; high blood pressure; tiredness; anemia; weight loss; an abnormal temperature (e.g., elevated); a lung condition/disease; a kidney condition/disorder; a liver condition/disorder; an ocular disorder/condition; a skin disorder/condition; an intestinal disorder/condition; and an infection.

Administration of the binding protein to the subject in various embodiments of the method improves a score of one or more rheumatoid arthritis metrics in the subject. For example, the rheumatoid arthritis metric is selected from the group consisting of: Physician Global Assessment of Disease Activity; Patient Reported Outcome; a Health Assessment Questionnaire (HAQ-DI); a patient global assessment of disease activity (VAS)); measurement or presence of an anti-drug antibody (ADA); tender joint count (TJC); swollen joint count (SJC); patient's assessment of pain; Work Instability Scale for Rheumatoid Arthritis; Short Form Health Survey (SF-36); American College of Rheumatology, ACR, (e.g., ACR20, ACR50, and ACR70); proportion of subjects achieving Low Disease Activity (LDA); Disease Activity Score 28 (DAS28; e.g., DAS28 based on C-reactive protein); Clinical Disease Activity Index (CDAI); simple disease activity index (SDAI); and Clinical Remission criteria.

An aspect of the invention provides methods for treating a subject having RA, such that the subject is resistant to treatment with methotrexate, the method comprising the step of administering to the subject a composition comprising a binding protein that specifically binds both IL-17 and TNF-α, and the binding protein is a dual variable domain immunoglobulin (DVD-Ig™) protein, and the binding protein comprises at least one polypeptide comprising the amino acid sequence of SEQ ID NO: 4 and the amino acid sequence of SEQ ID NO:9, and the binding protein is administered at from about 50-400 milligrams of the binding protein. The binding protein in various embodiments of the method is administered every week. In various embodiments of the method, the binding protein is administered every other week. In various embodiments of the method, the binding protein is administered intravenously. The binding protein in various embodiments of the method is administered subcutaneously.

In various embodiments, the method further comprises administering the composition including the binding protein after the methotrexate. Alternatively, the method further comprises administering the composition including the binding protein prior or currently with the methotrexate.

The binding protein in various embodiments of the method is administered at a dosage of about: 0.1 milligram per kilogram of subject weight (mg/kg); 0.3 mg/kg; 1.0 mg/kg; 3 mg/kg; and 10 mg/kg. The composition in various embodiments of the method further comprises at least one substance selected from the group consisting of sucrose, histidine, polysorbate, and mineral acid.

This invention pertains to the administration of binding proteins, or antigen-binding portions thereof, that bind IL-17 or TNF-α, such as DVD-Ig™ binding proteins that bind IL-17 and TNF-α for the treatment of RA. Various aspects of the invention relate to the use of bi-specific antibodies and antibody fragments thereof, DVD-Ig™| binding proteins, and pharmaceutical compositions thereof, as well as nucleic acids, recombinant expression vectors and host cells for making such IL-17/TNF binding proteins. Methods of using the IL-17/TNF binding proteins to detect human IL-17A homodimer and/or IL-17A/F heterodimer, either in vitro or in vivo, and to regulate gene expression are also encompassed by the methods of the invention. The methods of the invention also encompass the use of any binding protein or antibody capable of competing with an IL-17/TNF-α binding protein described herein. In certain embodiments, the binding protein is a DVD-Ig™ binding protein comprising one or more of the sequences shown in Example 1.

### DEFINITIONS

Unless otherwise defined herein, scientific and technical terms used herein shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. The use of “or” means “and/or” unless stated otherwise. Furthermore, the use of the term “including”, as well as other forms, such as “includes” and “included”, is not limiting.

Generally, nomenclatures and techniques used in connection with cell and tissue culture, molecular biology, immunology, microbiology, genetics and protein and nucleic acid chemistry and hybridization described herein are those well-known and commonly used in the art and are described in various general and more specific references that are cited and discussed throughout the specification unless otherwise indicated. Enzymatic reactions and purification techniques are performed according to manufacturer's specifications, as commonly performed in the art or otherwise as described herein. The nomenclatures and techniques used in connection with analytical chemistry, synthetic organic chemistry, and medicinal and pharmaceutical chemistry described herein are those well-known and commonly used in the art and are described in various general and more specific references that are cited and discussed throughout the specification unless otherwise indicated. Standard techniques are used for chemical syntheses, chemical analyses, pharmaceutical preparation, formulation, and delivery, and treatment of patients. For example, formulations and methods of producing and making compositions using a binding protein (e.g., a DVD-Ig™ protein) are described in U.S. 20140161817; US 20100266531; and US 20140017246, each of which is incorporated by reference herein in its entirety.

Select terms are defined below.

The term “adalimumab” means a recombinant human immunoglobulin (IgG1)monoclonal antibody containing only human peptide sequences. Adalimumab is produced by recombinant DNA technology in a mammalian cell expression system. It consists of 1330 amino acids and has a molecular weight of approximately 148 kilodaltons. Adalimumab is composed of fully human heavy and light chain variable regions, which confer specificity to human TNF, and human IgG1 heavy chain and kappa light chain sequences. Adalimumab binds with high affinity and specificity to soluble TNF-α but not to lymphotoxin-α (TNF-β) The terms “tumor necrosis factor” and “TNF” mean a naturally occurring cytokine that is involved in normal inflammatory and immune responses. Elevated levels of TNF play an important role in pathologic inflammation. Adalimumab binds specifically to TNF and neutralizes the biological function of TNF by blocking its interaction with the p55 and p75 cell surface TNF receptors. Adalimumab also modulates biological responses that are induced or regulated by TNF. After treatment with adalimumab, levels of acute phase reactants of inflammation (C-reactive protein [CRP] and erythrocyte sedimentation rate [ESR]) and serum cytokines rapidly decrease.

The term “biological activity” means all inherent biological properties of a molecule.

A “disease-modifying anti-rheumatic drug” (DMARD) means a drug or agent that modulates, reduces or treats the symptoms and/or progression associated with an immune system disease, including autoimmune diseases (e.g., rheumatic diseases), graft-related disorders and immunoproliferative diseases. The DMARD may be a synthetic DMARD (e.g., a conventional synthetic disease modifying antirheumatic drug) or a biologic DMARD. For example, the DMARD used may be a methotrexate, a sulfasalazine (Azulfidine), a cyclosporine (Neoral®, Sandimmune®), a leflunomide (Arava®), a hydroxychloroquine (Plaquenil®), a Azathioprine (Imuran®), or a combination thereof. In various embodiments, a DMARD is used to treat or control progression, joint deterioration, and/or disability associated with RA.

The term “polypeptide” means any polymeric chain of amino acids and encompasses native or artificial proteins, polypeptide analogs or variants of a protein sequence, or fragments thereof, unless otherwise contradicted by context. A polypeptide may be monomeric or polymeric. For an antigenic polypeptide, a fragment of a polypeptide optionally contains at least one contiguous or nonlinear epitope of a polypeptide. The precise boundaries of the at least one epitope fragment can be confirmed using ordinary skill in the art.

The term “variant” means a polypeptide that differs from a given polypeptide in amino acid sequence by the addition, deletion, or conservative substitution of amino acids, but that retains the biological activity of the given polypeptide (e.g., a variant TNF-α can compete with anti-TNFα antibody for binding to TNF). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al. (1982) J. Mol. Biol. 157:105-132). The hydrophilicity of amino acids also can be used to identify substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” encompasses a polypeptide or fragment thereof that has been differentially processed, such as by proteolysis, phosphorylation, or other post-translational modification, yet retains its biological activity or antigen reactivity, e.g., the ability to bind to TNF-α and IL-17. The term “variant” encompasses fragments of a variant unless otherwise contradicted by context.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a protein or polypeptide that is chemically synthesized or synthesized in a cellular system different from the cell from which it naturally originates is isolated from its naturally associated components. A protein or polypeptide may also be rendered substantially free of naturally associated components by isolation using protein purification techniques well known in the art.

The term “human IL-17” (“hIL-17”) includes a homodimeric protein comprising two 15 kD IL-17A proteins (hIL-17A/A) and a heterodimeric protein comprising a 15 kD IL-17A protein and a 15 kD IL-17F protein (“hIL-17A/F”). The amino acid sequences of hIL-17A and hIL-17F are shown in Table 1. The term “hIL-17” includes recombinant hIL-17 (rhIL-17), which can be prepared by standard recombinant expression methods.

TABLE 1
Sequence of Human IL-17A and Human IL-17F

| | Sequence | Sequence |
|---|---|---|
| Protein | Identifier | 12345678901234567890123456789012 |
| Human | SEQ ID | GITIPRNPGCPNSEDKNFPRTVMVNLNIHNRN |
| IL-17A | NO.: 1 | TNTNPKRSSDYYNRSTSPWNLHRNEDPERYPS |
| | | VIWEAKCRHLGCINADGNVDYHMNSVPIQQEI |
| | | LVLRREPPHCPNSFRLEKILVSVGCTCVTPIV |
| | | HHVA |
| Human | SEQ ID | RKIPKVGHTFFQKPESCPPVPGGSMKLDIGII |
| IL-17F | NO.: 2 | NENQRVSMSRNIESRSTSPWNYTVTWDPNRYP |
| | | SEVVQAQCRNLGCINAQGKEDISMNSVPIQQE |
| | | TLVVRRKHQGCSVSFQLEKVLVTVGCTCVTPV |
| | | IHHVQ |

The phrase “IL-17/TNF-α binding protein” means a bispecific binding protein (e.g., DVD-Ig™| protein) that binds IL-17 and TNF-α. The relative positions of the TNF-α binding region and IL-17 binding region within the bispecific binding protein are not fixed (e.g., VD1 or VD2 of the DVD-Ig™| protein) unless specifically specified herein.

The term “human TNF-α” (“hTNF-α”, or simply “hTNF”) means a 17 kD secreted form and a 26 kD membrane associated form of a human cytokine, the biologically active form of which is composed of a trimer of noncovalently bound 17 kD molecules. The structure of hTNF-α is described further in, for example, Pennica et al. (1984) Nature 312:724-729; Davis et al. (1987) Biochem. 26:1322-1326; and Jones et al. (1989) Nature 338:225-228. The term hTNF-α includes recombinant human TNF-α (“rhTNF-α”). The amino acid sequence of hTNF-α is shown in Table 2.

TABLE 2
Sequence of Human TNF-α

| | Sequence | Sequence |
|---|---|---|
| Protein | Identifier | 12345678901234567890123456789012 |
| Human | SEQ ID | MSTESMIRDVELAEEALPKKTGGPQGSRRCLF |
| TNF-α | NO.: 3 | LSLFSFLIVAGATTLFCLLHFGVIGPQREEFP |
| | | RDLSLISPLAQAVRSSSRTPSDKPVAHVVANP |
| | | QAEGQLQWLNRRANALLANGVELRDNQLVVPS |
| | | EGLYLIYSQVLFKGQGCPSTHVLLTHTISRIA |
| | | VSYQTKVNLLSAIKSPCQRETPEGAEAKPWYE |
| | | PIYLGGVFQLEKGDRLSAEINRPDYLDFAESG |
| | | QVYFGIIAL |

The terms “specific binding” or “specifically binding”, in reference to the interaction of an antibody, a protein, or a peptide with a second chemical species, mean that the interaction is dependent upon the presence of a particular structure (e.g., an antigenic determinant or epitope) on the chemical species. If an antibody is specific for epitope “A”, in the presence of a molecule containing epitope A (or free, unlabeled epitope A) in which “A” is labeled, the antibody reduces the amount of labeled A bound to the antibody. “Specific binding partner” is a member of a specific binding pair. The term “specific binding pair” comprises two different molecules, which specifically bind to each other through chemical or physical means (e.g., an antigen (or fragment thereof) and an antibody (or antigenically reactive fragment thereof)). Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes, fragments, and variants (including fragments of variants) thereof, whether isolated or recombinantly produced. The terms “specific” and “specificity” in the context of an interaction between members of a specific binding pair refer to the selective reactivity of the interaction.

The term “human antibody” includes antibodies having variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” means human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “CDR” means the complementarity determining region within antibody variable sequences. There are three CDRs in each of the variable regions of the heavy chain and the light chain, which are designated CDR1, CDR2, and CDR3, for each of the variable regions. The term “CDR set” means a group of three CDRs that occur in a single variable region (i.e., VH or VL) of an antigen binding site. The exact boundaries of these CDRs have been defined differently according to different systems. The system described by Kabat (Kabat et al. (1987, 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196: 901-917 and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2, and L3 or H1, H2, and H3, where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139 and MacCallum (1996) J. Mol. Biol. 262(5): 732-745). Still other CDR boundary definitions may not strictly follow one of the above systems, but nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The terms “Kabat numbering,” “Kabat definition,” and “Kabat labeling” mean a system of numbering amino acid residues which are more variable (i.e., hypervariable) than other amino acid residues in the heavy and light chain variable regions of an antibody, or an antigen binding portion thereof (Kabat et al. (1971) Ann NY Acad. Sci. 190: 382-391 and Kabat et al. (1991) “Sequences of Proteins of Immunological Interest, Fifth Edition”, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The growth and analysis of extensive public databases of amino acid sequences of variable heavy and light regions over the past twenty years have led to the understanding of the typical boundaries between framework regions (FR) and CDR sequences within variable region sequences and enabled persons skilled in this art to accurately determine the CDRs according to Kabat numbering, Chothia numbering, or other systems. See, e.g., Martin, “Protein Sequence and Structure Analysis of Antibody Variable Domains,” In Kontermann and Diibel, eds., Antibody Engineering (Springer-Verlag, Berlin, 2001), chapter 31, pages 432-433. A useful method of determining the amino acid sequences of Kabat CDRs within the amino acid sequences of variable heavy (VH) and variable light (VL) regions is provided below:

To identify a CDR-L1 amino acid sequence:
Starts approximately 24 amino acid residues from the amino terminus of the
VL region;
Residue before the CDR-L1 sequence is always cysteine (C);
Residue after the CDR-L1 sequence is always a tryptophan (W) residue,
typically Trp-Tyr-Gln (W-Y-Q), but also Trp-Leu-Gln (W-L-Q), Trp-Phe-Gln (W-F-Q), and Trp-Tyr-Leu (W-Y-L);
Length is typically 10 to 17 amino acid residues.

To identify a CDR-L2 amino acid sequence:
Starts always 16 residues after the end of CDR-L1;
Residues before the CDR-L2 sequence are generally Ile-Tyr (I-Y), but also
Val-Tyr (V-Y), Ile-Lys (I-K), and Ile-Phe (I-F);
Length is always 7 amino acid residues.

To identify a CDR-L3 amino acid sequence:
Starts always 33 amino acids after the end of CDR-L2;
Residue before the CDR-L3 amino acid sequence is always a cysteine (C);
Residues after the CDR-L3 sequence are always Phe-Gly-X-Gly (F-G-X-G)
(SEQ ID NO:47), where X is any amino acid;
Length is typically 7 to 11 amino acid residues.

To identify a CDR-H1 amino acid sequence:
Starts approximately 31 amino acid residues from amino terminus of VH
region and always 9 residues after a cysteine (C);
Residues before the CDR-H1 sequence are always Cys-X—X-X-X—X-X-X-X
(SEQ ID NO: 48), where X is any amino acid;
Residue after CDR-H1 sequence is always a Trp (W), typically Trp-Val (W-V), but also Trp-Ile (W-I), and Trp-Ala (W-A);
Length is typically 5 to 7 amino acid residues.

To identify a CDR-H2 amino acid sequence:
Starts always 15 amino acid residues after the end of CDR-H1;
Residues before CDR-H2 sequence are typically Leu-Glu-Trp-Ile-Gly (L-E-W-I-G) (SEQ ID NO: 51), but other variations also;
Residues after CDR-H2 sequence are Lys/Arg-Leu/Ile/Val/Phe/Thr/Ala-Thr/Ser/Ile/Ala (K/R-L/I/V/F/T/A-T/S/I/A);
Length is typically 16 to 19 amino acid residues.

To identify a CDR-H3 amino acid sequence:
Starts always 33 amino acid residues after the end of CDR-H2 and always 3
after a cysteine (C)′
Residues before the CDR-H3 sequence are always Cys-X-X (C—X-X), where
X is any amino acid, typically Cys-Ala-Arg (C-A-R);
Residues after the CDR-H3 sequence are always Trp-Gly-X-Gly (W-G-X-G)
(SEQ ID NO: 52), where X is any amino acid;
Length is typically 3 to 25 amino acid residues.

With respect to constructing DVD-Ig or other binding protein molecules, the term “linker” means a single amino acid or a polypeptide comprising two or more amino acid residues joined by peptide bonds (“linker polypeptide”) used to link one or more antigen binding portions. Such linker polypeptides are well known in the art (see, e.g., Holliger et al., (1993) Proc. Natl. Acad. Sci. USA, 90: 6444-6448; Poljak (1994) Structure, 2: 1121-1123). Exemplary linkers include, but are not limited to, GGGGSG (SEQ ID NO:14), GGSGG (SEQ ID NO:15), GGGGSGGGGS (SEQ ID NO:16), GGSGGGGSG (SEQ ID NO:17), GGSGGGGSGS (SEQ ID NO:18), GGSGGGGSGGGGS (SEQ ID NO:19), GGGGSGGGGSGGGG (SEQ ID NO:20), GGGGSGGGGSGGGGS (SEQ ID NO:21), ASTKGP (SEQ ID NO:22), ASTKGPSVFPLAP (SEQ ID NO:23), TVAAP (SEQ ID NO:24), RTVAAP (SEQ ID NO:25), TVAAPSVFIFPP (SEQ ID NO:26), RTVAAPSVFIFPP (SEQ ID NO:27), AKTTPKLEEGEFSEAR (SEQ ID NO:28), AKTTPKLEEGEFSEARV (SEQ ID NO:29), AKTTPKLGG (SEQ ID NO:30), SAKTTPKLGG (SEQ ID NO:31), SAKTTP (SEQ ID NO:32), RADAAP (SEQ ID NO:33), RADAAPTVS (SEQ ID NO:34), RADAAAAGGPGS (SEQ ID NO:35), RADAAAAGGGGSGGGGSGGGGSGGGGS (SEQ ID NO:36), SAKTTPKLEEGEFSEARV (SEQ ID NO:37), ADAAP (SEQ ID NO:38), ADAAPTVSIFPP (SEQ ID NO:39), QPKAAP (SEQ ID NO:40), QPKAAPSVTLFPP (SEQ ID NO:41), AKTTPP (SEQ ID NO:42), AKTTPPSVTPLAP (SEQ ID NO:43), AKTTAP (SEQ ID NO:44), AKTTAPSVYPLAP (SEQ ID NO:45), GENKVEYAPALMALS (SEQ ID NO:46), GPAKELTPLKEAKVS (SEQ ID NO:49), and GHEAAAVMQVQYPAS (SEQ ID NO:50).

The term “neutralizing” mean to render inactive activity, e.g., the biological activity of an antigen (e.g., the cytokines TNF-α and IL-17) when a binding protein specifically binds the antigen. Preferably, a neutralizing binding protein described herein binds to human TNF-α and/or human IL-17 resulting in the inhibition of a biological activity of the cytokines. Preferably, the neutralizing binding protein binds TNF-α and IL-17 and reduces a biologically activity of TNF-α and IL-17 by at least about 20%, 30%, 40%, 50%, 60%, 70%, 80%, 85%, 90%, 95%, or more. Inhibition of a biological activity of TNF-α and IL-17 by a neutralizing binding protein can be assessed by measuring one or more indicators of TNF-α and IL-17 biological activity well known in the art.

The term “activity” includes activities such as the binding specificity/affinity of an antibody for an antigen, for example, an anti-TNF-α and/or anti-IL-17 (e.g., hTNF-α and hIL-17) antibody that binds to TNF-α and/or IL-17.

The term “epitope” means a polypeptide determinant capable of specific binding to an immunoglobulin or T-cell receptor. In certain embodiments, epitope determinants include chemically active surface groupings of molecules such as amino acids, sugar side chains, phosphoryl or sulfonyl groups, and, in certain embodiments, may have specific three dimensional structural characteristics and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. Antibodies are said to bind to the same epitope if the antibodies cross-compete (one prevents the binding or modulating effect of the other). In addition, structural definitions of epitopes (overlapping, similar, identical) are informative, but functional definitions are often more relevant as they encompass structural (binding) and functional (modulation, competition) parameters.

The term “percent identity” means a quantitative measurement of the similarity between two sequences (complete amino acid sequence or a portion thereof). Calculations of sequence identity between sequences are known by those in the art. For example, to determine the percent identity of two amino acid sequences, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences. For example, percent identity can about 30%, 35%, 40%, 45%, 50%, 55%, 60%, 65%, 70%, 75%, 80%, 85%, 90%, 95%, 96%, 98%, 99%, or 99% or more.

The comparison of sequences and determination of percent identity between two sequences are accomplished using a mathematical algorithm. Percent identity between two amino acid sequences is determined using an alignment software program using the default parameters. Suitable programs include, for example, CLUSTAL W (see Thompson et al. (1994) Nucl. Acids Res. 22: 4673-4680) or CLUSTAL X.

The term “substantially identical” in reference to amino acid sequences means a first amino acid sequence that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 30%, 35%, 40%, 45%, 50%, 55%, 60%, 65%, 70%, 75%, 80%, 85%, 90%, 95%, 96%, 98%, 99%, or 99% or more identity to a DVD-Ig binding protein described herein (e.g., a DVD-Ig binding protein comprising SEQ ID NO: 4, SEQ ID NO: 9, or a portion or combination thereof). In various embodiments, the substantially identical protein includes an amino acid sequence that is at least about 30%, about 35%, about 40%, about 45%, about 50%, about 55%, about 60%, about 65%, about 70%, about 75%, about 80%, about 85%, about 90%, about 95%, about 99%, or 99% or more identical to SEQ ID NO: 4, SEQ ID NO: 9, or a portion or a combination thereof.

The term “surface plasmon resonance” means an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson et al. (1993) Ann. Biol. Clin. 51: 19-26; Jonsson et al. (1991), BioTechniques 11: 620-627; Johnsson et al. (1995) J. Mol. Recognit. 8: 125-131; and Johnsson et al. (1991) Anal. Biochem. 198: 268-277.

The terms“Kₒₙ” “Kon,” and “kon” mean the on rate constant for association or “association rate constant,” of a binding protein (e.g., an antibody) to an antigen to form an association complex, e.g., antibody/antigen complex, as is known in the art. The term “Kₒₙ” also is known by the terms “association rate constant” or “ka”. This value indicates the binding rate of an antibody to its target antigen or the rate of complex formation between an antibody and antigen as is shown by the equation below:

Antibody (“Ab”)+Antigen (“Ag”)→Ab−Ag

The terms “K_{off},” “Koff,” and “koff” mean the off rate constant for dissociation, or “dissociation rate constant,” of a binding protein (e.g., an antibody) from an association complex (e.g., an antibody/antigen complex) as is known in the art. This value indicates the dissociation rate of an antibody from its target antigen or separation of Ab-Ag complex over time into free antibody and antigen as shown by the equation below:

Ab+Ag←Ab−Ag

The terms “K_{D}” and “K_{d}”, and the “equilibrium dissociation constant,” and mean o the value obtained in a titration measurement at equilibrium, or by dividing the dissociation rate constant (Koff) by the association rate constant (Kon). The association rate constant (Kon), the dissociation rate constant (Koff), and the equilibrium dissociation constant (K are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used. Additionally, a KinExA® (Kinetic Exclusion Assay) assay, available from Sapidyne Instruments (Boise, Id.) can also be used.

The terms “AUC” and “area under the curve” mean the area under the plasma drug concentration-time curve, and reflects the actual body exposure to drug after administration of a dose of the drug. AUC is typically related to clearance. A higher clearance rate is related to a smaller AUC, and a lower clearance rate is related to a larger AUC value. The AUC higher values represent slower clearance rates.

The term “volume of distribution” means the theoretical volume of fluid into which the total drug administered would have to be diluted to produce the concentration in plasma. Calculating the volume of distribution may in various embodiments involve the quantification of the distribution of a drug, e.g., a TNFα/IL-17 DVD-Ig binding protein, or antigen-binding portion thereof, between plasma and the rest of the body after dosing. The volume of distribution is the theoretical volume in which the total amount of drug would need to be uniformly distributed in order to produce the desired blood concentration of the drug.

The terms “half-life” and “T1/2” mean the time for half of a drug's concentration or activity (e.g., pharmacologic or physiologic) to be measurable compared to a previously measured peak concentration or activity. In various embodiments, the quantification of the half-life may involve determining the time taken for half of the concentration or activity a dose of a drug to be measurable, e.g., in the blood, or other body fluid, in a subject or same over time. For example, the half-life may involve the time taken for half of the dose to be eliminated, excreted or metabolized.

The term “Cmax” means the peak concentration that a drug is observed, quantified or measured in a specified fluid or sample after the drug has been administrated. In various embodiments, determining the Cmax involves in part quantification of the maximum or peak serum or plasma concentration of a drug/therapeutic agent observed in a sample from a subject administered the drug.

The term “bioavailability” means the degree to which a drug is absorbed or becomes available to cells or tissue after administration of the drug. For example, bioavailability in certain embodiments involves quantification of the fraction or percent of a dose which is absorbed and enters the systemic circulation after administration of a given dosage form. See international publication number WO2013078135 published May 30, 2013, which is incorporated by reference herein in its entirety.

The terms “label” and “detectable label” mean a moiety attached to a specific binding partner, such as an antibody or an analyte, e.g., to render the reaction between two specific binding partners (specific binding pair) detectable. The specific binding partner so labeled is referred to as “detectably labeled”. Thus, the term “labeled binding protein” means a protein with a label incorporated that provides for the identification of the binding protein or the ligand to which it binds. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin or streptavidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm), chromogens, fluorescent labels (e.g., FITC, rhodamine, lanthanide phosphors), enzymatic labels (e.g., horseradish peroxidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), and magnetic agents (e.g., gadolinium chelates). Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety. Use of the term “detectably labeled” is intended to encompass the latter type of detectable labeling.

The term “binding protein conjugate” means a binding protein that is chemically linked to a second chemical moiety, such as a therapeutic or cytotoxic agent.

The term “agent” means a chemical compound, a mixture of chemical compounds, a biological macromolecule, or an extract made from biological materials. The therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, a binding protein conjugate may be a detectably labeled antibody, which is used as the detection antibody.

The terms “crystal” and “crystallized” mean an agent in the form of a crystal. Crystals are one form of the solid state of matter that is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. See Giege et al., Chapter 1, In Crystallization of Nucleic Acids and Proteins, a Practical Approach, 2nd ed., (Ducruix and Giege, eds.) (Oxford University Press, New York, 1999) pp. 1-16.

The term “polynucleotide” means a polymer of two or more nucleotides, e.g., ribonucleotides or deoxynucleotides or a modified form of nucleotide. The term includes single and double stranded forms of DNA.

The term “isolated polynucleotide” means a polynucleotide (e.g., of genomic, cDNA, or synthetic origin, or some combination thereof) that, by virtue of its origin, is not associated with all or a portion of a polynucleotide with which the polynucleotide is found in nature; is operably linked to a polynucleotide that it is not linked to in nature; or does not occur in nature as part of a larger sequence.

The term “vector” means a nucleic acid molecule capable of transporting another nucleic acid to which it has been linked One type of vector is a “plasmid”, which refers to a circular double stranded DNA loop into which additional DNA segments may be ligated. Another type of vector is a viral vector, wherein additional nucleic acid segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked (“recombinant expression vectors” or “expression vectors”). In general, expression vectors are often in the form of plasmids. Vectors may also be viral vectors (e.g., replication defective retroviruses, adenoviruses and adeno-associated viruses).

The term “operably linked” refers to a juxtaposition wherein the components described are in a relationship permitting them to function in their intended manner. A control sequence that is “operably linked” to a coding sequence is ligated in such a way that expression of the coding sequence is achieved under conditions compatible with the control sequences. Operably linked sequences include both expression control sequences that are contiguous with the gene of interest and expression control sequences that act in trans or at a distance to control the gene of interest. The term “expression control sequence” means a polynucleotide sequence that is necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., Kozak consensus sequence); sequences that enhance protein stability; and sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequence” means a sequence whose presence is essential for expression and processing, and can also include additional components whose presence is advantageous, for example, leader sequences and fusion partner sequences.

The term “transformation” means a process by which exogenous DNA enters a host cell. Transformation may occur under natural or artificial conditions using various methods well known in the art. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells that transiently express the inserted DNA or RNA for limited periods of time.

The terms “recombinant host cell” and “host cell” mean a cell into which exogenous DNA has been introduced. In an embodiment, the host cell comprises two or more (e.g., multiple) nucleic acids encoding antibodies. Such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term host cell. In an embodiment, host cells include prokaryotic and eukaryotic cells selected from any of the Kingdoms of life. In another embodiment, eukaryotic cells include protist, fungal, plant and animal cells. In another embodiment, host cells include but are not limited to the prokaryotic cell line Escherichia coli; mammalian cell lines CHO, HEK 293, COS, NS0, SP2 and PER.C6; the insect cell line Sf9; and the fungal cell Saccharomyces cerevisiae.

Standard techniques may be used for recombinant DNA, oligonucleotide synthesis, tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual, 2nd ed. (Cold Spring Harbor Laboratory Press, Cold Spring Harbor, N.Y., 1989).

The term “modulator” means a compound capable of changing or altering an activity or function of a molecule of interest (e.g., the biological activity of hTNF-α and hIL-17). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in PCT Publication No. WO 01/83525.

The term “agonist” means a modulator that, when contacted with a molecule of interest, causes an increase in the magnitude of a certain activity or function of the molecule compared to the magnitude of the activity or function observed in the absence of the agonist. Particular agonists of interest may include, but are not limited to, TNF-α and IL-17 polypeptides, nucleic acids, carbohydrates, or any other molecule that binds to hTNF-α and hIL-17.

The terms “antagonist” and “inhibitor” mean a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of human TNF-α and IL-17. Antagonists and inhibitors of human TNF-α and IL-17 may include, but are not limited to, proteins, nucleic acids, carbohydrates, or any other molecules, which bind to human TNF-α and IL-17.

The term “effective amount” means the amount of a therapy that is sufficient to reduce or ameliorate the severity and/or duration of a disorder or one or more symptoms thereof; prevent the advancement of a disorder; cause regression of a disorder; prevent the recurrence, development, onset, or progression of one pr more symptoms associated with a disorder; detect a disorder; or enhance or improve the prophylactic or therapeutic effect(s) of another therapy (e.g., prophylactic or therapeutic agent).

The terms “patient” and “subject” mean an animal, such as a mammal, including a primate (for example, a human, a monkey, and a chimpanzee), a non-primate (for example, a cow, a pig, a camel, a llama, a horse, a goat, a rabbit, a sheep, a hamster, a guinea pig, a cat, a dog, a rat, a mouse, a whale), a bird and a fish. In an embodiment, the patient or subject is a human, such as a human being treated or assessed for a disease, disorder or condition; a human at risk for a disease, disorder or condition; and/or a human having a disease, disorder or condition.

The term “sample” means a quantity of a substance. The term “biological sample means a quantity of a substance from a living thing or formerly living thing. Such substances include, but are not limited to, blood, plasma, serum, urine, amniotic fluid, synovial fluid, endothelial cells, leukocytes, monocytes, other cells, organs, tissues, bone marrow, lymph nodes and spleen.

The term “component” means a portion of a mixture, composition, system or kit, for example a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), an analyte, a stop solution, and the like that can be included in a kit for assay of a test sample, such as a patient urine, serum or plasma sample, in accordance with the methods described herein and other methods known in the art. Some components can be in solution or lyophilized for reconstitution for use in an assay.

The term “control” means a component or composition that is not, or does not contain, an analyte (“negative control”) or is or contains analyte (“positive control”). A positive control can comprise a known concentration of analyte. A “calibrator” means a composition comprising a known concentration of analyte. A positive control can be used to establish assay performance characteristics and is a useful indicator of the integrity of reagents (e.g., analytes).

The terms “predetermined cutoff” and “predetermined level” mean an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). While the present disclosure may provide exemplary predetermined levels, cutoff values may vary depending on the nature of the immunoassay (e.g., antibodies employed). It is well within the ordinary skill of one in the art to adapt the disclosure herein for other immunoassays to obtain immunoassay-specific cutoff values for those other immunoassays. Whereas the precise value of the predetermined cutoff/level may vary between assays, correlations as described herein (if any) should be generally applicable.

The term “risk” means the possibility or probability of a particular event occurring either presently or at some point in the future. The term “risk stratification” means an array of known clinical risk factors that allows physicians to classify patients into a low, moderate, high or highest risk of developing a particular disease, disorder or condition.

The terms “DMARD resistance” and “resistance to a DMARD” means an observed or demonstrated loss of efficacy over time to treatment of a disorder (e.g., RA) using a DMARD. DMARDs resistance may be a multifactorial event including enhanced drug efflux via ABC transporters, impaired drug uptake and drug activation, enhanced drug detoxification etc. In various embodiments, the subject is observed to have a RA symptom that is not reduced by DMARD treatment.

The term “antibody” means any immunoglobulin (Ig) molecule comprised of four polypeptide chains, two heavy (H) chains and two light (L) chains, or any functional fragment, mutant, variant, or derivative thereof, which retains the essential epitope binding features of an Ig molecule. Such mutant, variant, or derivative antibody formats are known in the art, non-limiting embodiments of which are discussed below.

A number of abbreviations are used herein to describe aspects of the invention. Below is a list of commonly used abbreviations.
ACR—American College of Rheumatology
ADA—Anti-drug antibody
AE—Adverse event
ALT—Alanine aminotransferase
ANC—Absolute neutrophil count
AUC—Area under the serum concentration-time curve; e.g., (μg·hr/mL or mg·hr/mL)
BA—Bioavailability
BQL—Below quantitation limit
BUN—Blood Urea Nitrogen
Cl/F—Apparent clearance
C1M—Matrix metalloproteinase-mediated degradation of type I collagen
C2M—Matrix metalloproteinase-mediated degradation of type II collagen
C3M—Matrix metalloproteinase-mediated degradation of type III collagen
CD—Crohn's disease
CDAI—Clinical Disease Activity Index
CH50—50% hemolytic complement activity (assay)
CIA—Collagen-induced arthritis
CIC—Circulating immune complex
Cmax—Maximum observed serum concentration
COX—Cyclooxygenase
CR—Clinical Remission
CRPM—Matrix metalloproteinase-mediated C-reactive protein
Ctrough—Trough concentration; lowest concentration of the drug in the blood that is measured after a dose
CTX-I—C-terminal telopeptide type I collagen
CTX-II—C-terminal telopeptide type II collagen
DAS-28—Disease activity score 28
DB—Double-blind
DMARD—Disease-modifying antirheumatic drug
DR—Disease Response
DVD-Ig™—Dual-variable domain immunoglobulin
ECG—Electrocardiogram
eCRF—Electronic case report form
ED50—Dose required to produce a 50% reduction in response
EDC—Electronic data capture
ELISA—Enzyme-linked immunosorbent assay
EOW—Every other week
ESRB—External Safety Review Board
EULAR—European League against Rheumatism
EW—Every Week
F—Bioavailability
FACIT-F—Functional Assessment of Chronic Illness Therapy-Fatigue
FIH—First-in-human
FITC—Fluorescein isothiocyanate
GCP—Good Clinical Practice
GLP—Good Laboratory Practice
HAQ-DI—Health Assessment Questionnaire Disability Index
Hrs—Hours
hsCRP—High sensitivity C-reactive protein
IC50—Inhibitory concentration 50 percent
ICH—International Conference on Harmonisation
IEC—Independent Ethics Committee
IgG—Immunoglobulin G
IgG1—Immunoglobulin G1
IHC—Immunohistochemical
IL—Interleukin
IL-17—Interleukin 17
IP—Intraperitoneal
IRB—Institutional Review Board
IUD—Intrauterine Device
IV—Intravenous(ly)
IVRS—Interactive voice response system
IWRS—Interactive web response system
JAK—Janus kinase
KC—Keratinocyte-derived chemokine
KD—Dissociation constant
LDA—Low Disease Activity
mAb—Monoclonal antibody
MAD—Multiple ascending dose
MAS—Mean arthritic score
MedDRA—Medical Dictionary for Regulatory Activities
mg/kg—Milligrams per kilogram
micro-CT—Micro-computed tomography
MMP—Matrix metalloproteinases
MMP-3—Matrix metalloproteinase 3
MRNA—Messenger ribonucleic acid
MRT—Mean residence time
MSD—Meso Scale Discovery
MTX—Methotrexate
NA—Not applicable
NOAEL—No-observed-adverse-effect-level
NSAID—Nonsteroidal anti-inflammatory drugs
OLE—Open-Label Extension
PD—Premature Discontinuation or Pharmacodynamic
PDR—Post-dose reaction
PEF—Peak Expiratory Flow
PGA—Physician's Global Assessment of Disease Activity
PK—Pharmacokinetic(s)
PT—Preferred term
PtGA—Patient's Global Assessment of Disease Activity
RA—Rheumatoid arthritisRA-WIS—Rheumatoid Arthritis Work Instability Scale
RBC—Red blood cells
RCT—Randomized Controlled Trial
rIL-17—Recombinant interleukin-17
rTNF—Recombinant tumor necrosis factor
SAD—Single ascending dose
SAE—Serious adverse event
SC—Subcutaneous(ly)
SCR—Screening
SD—Standard deviation
SF-36v2—Short form health surveySGPT/ALT—Serum glutamic-pyruvic transaminase
SGOT/AST—Serum glutamic-oxaloacetic transaminase
SJC—Swollen joint count
SOC—System organ class
SUSAR—Suspected unexplained serious adverse reaction
TB—Tuberculosis
TJC—Tender joint count
Tmax—Time to reach maximum concentration
TNF—Tumor necrosis factor
t1/2—Terminal phase elimination half-life
μg/mL—Micrograms per milliliter
ULN—Upper limit of normal
VAS—Visual analog scale
VICM—Citrullinated and matrix metalloproteinase—degraded vimentin
Vss—Volume of distribution
Vss/F—Volume of distribution at steady-state
WBC—White blood cell
Pharmacokinetic and Statistical Abbreviations
ANCOVA Analysis of covariance
AUC Area under the curve
AUC_{∞} Area under the curve from time zero to infinity
AUCt Area under curve from time zero to time t
C Concentration
Ct Concentration at a specified time t after the administration of a dose
CL/F Apparent clearance
Cmax Maximum observed plasma concentration
Ctrough Observed serum concentration at the end of dosing interval
ECDF Empirical cumulative distribution function
F Bioavailability
ITT Intent-to-treat
KS Kolmogorov-Smirnov
LLQ Lower limit of quantification
LOCF Last observation carried forward
IR Insufficient responder
MAT Mean absorption time
NR Non-responder
NRI Non-responder imputation
OC Observed cases
pKa Acid dissociation constant at logarithmic scale
Rac Accumulation ratio
Rac(AUC) Accumulation ratio calculated from AUCτ,ss and AUCτ after single dosing
t1/2 half-life
tabs Absorption half-life
Tmax Time to maximum observed plasma concentration
ULQ Upper limit of quantification
V/F Apparent volume of distribution

### Anaphylaxis Abbreviations

1. Acute onset of an illness (minutes to several hours) with involvement of the skin, mucosal tissue, or both (e.g., generalized hives, pruritus or flushing, swollen lips-tongue-uvula) and at least one of the following: a. Respiratory compromise (e.g., dyspnea, wheeze bronchospasm, stridor, reduced peak expiratory flow, hypoxemia). b. Reduced BP or associated symptoms or end-organ dysfunction (e.g., hypotonia [collapse], syncope, incontinence).
2. Two or more of the following that occur within minutes to several hours to study drug. a. Involvement of the skin-mucosal tissue (e.g., generalized hives, itch-flush, swollen lips tongue-uvula). b. Respiratory compromise (e.g., dyspnea, wheeze-bronchospasm, stridor, reduced PEF, hypoxemia). c. Reduced BP or associated symptoms (e.g., crampy abdominal pain, vomiting). d. Persistent gastrointestinal symptoms (e.g., crampy abdominal pain, vomiting).
3. Reduced BP after exposure to study drug (within minutes to several hours), with systolic BP of less than 90 mmHg or greater than 30% decrease from that person's baseline. Sampson et al. (2006) J. Allergy Clin. Immunol. 117(2):391-7.

Serious Systemic Hypersensitivity Reaction: A hypersensitivity reaction is a clinical sign or symptom, or constellation of signs or symptoms, caused by an inappropriate and excessive immunologic reaction to study drug administration. A systemic hypersensitivity reaction is a hypersensitivity reaction that does not occur at the local site of study drug administration (e.g., not an injection site reaction). A serious systemic hypersensitivity reaction is a systemic hypersensitivity reaction that fulfills criteria for a serious adverse event.

It will be readily apparent to those skilled in the art that other suitable modifications and adaptations of the methods of the invention described herein are obvious and may be made using suitable equivalents without departing from the scope of the invention or the embodiments disclosed herein. The invention will be more clearly understood by reference to the following examples, which are included for purposes of illustration only and are not intended to be limiting.

### EXEMPLIFICATION

### Example 1

### Serum Exposure Analysis of a Repeat Dose Pharmacokinetic Study of a IL-17/TNF DVD-Ig Protein in Cynomolgus Monkeys

TNF and IL-17 are independently involved in pathophysiology of rheumatoid arthritis. Pre-clinical testing was performed using TNF antibodies and IL-17 antibodies. The corresponding data supported administering a combination of TNF antibodies and IL-17 antibodies in order to obtaining greater treatment efficacy. A mouse arthritis model was used to analyze arthritic score for 21 days for subjects treated with anti-TNF antibodies, anti-Il-17 antibodies, or a mixture of anti-TNF antibodies and anti-IL-17 antibodies. TNF-antibodies or IL-17 antibodies reduced arthritic scores compared to control subjects not administered the antibodies. Most importantly, dual inhibition of TNF and IL-17 using a mixture of antibodies provided greater efficacy against inflammation, and cartilage and bone erosion than treatment with anti-TNF antibody only or anti-IL-17 antibody only. In vitro studies of fibroblasts from rheumatoid arthritis patients indicated that TNF and IL-17 acted in synergy and induced mediators of inflammation and joint destruction.

ABT-122 is a human anti-human TNF/IL-17 dual variable domain immunoglobulin (DVD-Ig™) protein under evaluation for immunology indications. ABT-122 is a recombinant DVD-Ig comprised of two identical kappa (κ) light chains and two identical IgG1 heavy chains covalently attached through a full complement of inter—and intra-molecular disulfide bonds. The disulfide linkage pattern is structurally similar to that of natural IgG1 antibodies. The heavy chain is post—translationally modified by addition of N-linked glycans to the heavy chain at the same asparagine location commonly modified on IgG1 antibodies. The major glycans are fucosylated biantennary oligosaccharides containing zero, one or two galactose residues. Each light chain and heavy chain contains two variable domains connected in tandem by flexible glycine-serine peptide linker regions enabling dual specificity capable of binding both IL-17 and TNF-α in a tetravalent manner. Except for these linkers, the heavy chain and light chain variable and constant regions of ABT-122 have fully human amino acid sequences. ABT-122 DVD-Ig™ binding protein has a molecular weight of approximately 198.5 kilodaltons.

The ABT-122 drug substance is a DVD-Ig™ binding protein in a formulation buffer suitable for manufacturing the pharmaceutical dosage form. Solubility is 65 mg/ml at a minimum in formulation buffer. The dosage form is a powder for solutions for injection in a glass vial. The strength is 50/mg/mL and 100 mg/mL after reconstitution. Excipients include histidine, sucrose and polysorbate-80. The drug product, (ABT-122 powder for solution for injection, 50 and 100 mg/mL, in vials) was stored and refrigerated at 2° to 8° C. Vials were protected from light. The reconstitution of the lyophilizate vials and the preparation of dose solutions are described herein.

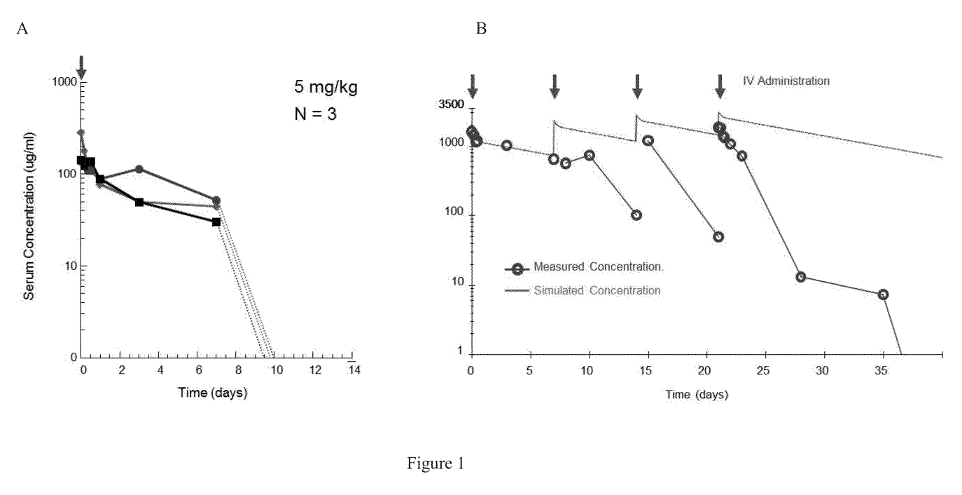

The dual neutralization of TNF and IL-17 may provide superior efficacy to the current standard of care treatments for rheumatoid arthritis and other inflammatory diseases. The pharmacokinetic study described herein was conducted to assess the pharmacokinetic profile and the effects of ABT-122 on cynomolgus (cyno) monkeys administered intravenously with a single dose at 5 mg/kg (FIG. 1, panel A) or administered intravenously at 45 mg/kg once weekly for four weeks (FIG. 1 panel B). Serum samples were collected throughout the study and serum ABT-122 concentrations were measured by biotinylated human TNF-α capture MSD assay. Pharmacokinetic parameters were calculated from serum test article concentrations using WinNonlin software with non-compartmental analysis. Following intravenous administration at 45 mg/kg in cyno, ABT-122 exposures were reduced from first to fourth dose. These reduced test article concentrations from first to fourth dose may be due to anti-drug antibody (ADA) formation.

The amino acid sequence of ABT-122 is provided in Table 3. CDRs for the variable domains are in bold.

TABLE 3
Heavy Variable Domain And Light Variable
Domain Amino Acid Sequences of ABT-122

| | | |
|---|---|---|
| DVD HEAVY | SEQ ID | EVQLVESGGGLVQPGRSLRL |
| VARIABLE | NO.: 4 | SCAASGFTFDDYAMHWVRQA |
| D2E7-GS10-B6-17 | | PGKGLEWVSAITWNSGHIDY |
| DVD-Ig Protein\| | | ADSVEGRFTISRDNAKNSLY |
| | | LQMNSLRAEDTAVYYCAKVS |
| | | YLSTASSLDYWGQGTLVTVS |
| | | SGGGGSGGGGSEVQLVQSGA |
| | | EVKKPGSSVKVSCKASGGSF |
| | | GGYGIGWVRQAPGQGLEWMG |
| | | GITPFFGFADYAQKFQGRVT |
| | | ITADESTTTAYMELSGLTSD |
| | | DTAVYYCARDPNEFWNGYYS |
| | | THDFDSWGQGTTVTVSS |
| D2E7 VH | SEQ ID | EVQLVESGGGLVQPGRSLRL |
| | NO.: 5 | SCAASGFTFDDYAMHWVRQA |
| | | PGKGLEWVSAITWNSGHIDY |
| | | ADSVEGRFTISRDNAKNSLY |
| | | LQMNSLRAEDTAVYYCAKVS |
| | | YLSTASSLDYWGQGTLVTVS |
| | | S |
| LINKER | SEQ ID | GGGGSGGGGS |
| | NO.: 6 | |
| B6-17 VH | SEQ ID | EVQLVQSGAEVKKPGSSVKV |
| | NO.: 7 | SCKASGGSFGGYGIGWVRQA |
| | | PGQGLEWMGGITPFFGFADY |
| | | AQKFQGRVTITADESTTTAY |
| | | MELSGLTSDDTAVYYCARDP |
| | | NEFWNGYYSTHDFDSWGQGT |
| | | TVTVSS |
| CH | SEQ ID | ASTKGPSVFPLAPSSKSTSG |
| | NO.: 8 | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPELLGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSRDE |
| | | LTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| DVD LIGHT | SEQ ID | DIQMTQSPSSLSASVGDRVT |
| VARIABLE | NO.: 9 | ITCRASQGIRNYLAWYQQKP |
| D2E7-GS10-B6- | | GKAPKLLIYAASTLQSGVPS |
| 17 DVD-Ig | | RFSGSGSGTDFTLTISSLQP |
| Protein\| | | EDVATYYCQRYNRAPYTFGQ |
| | | GTKVEIKRGGSGGGGSGEIV |
| | | LTQSPDFQSVTPKEKVTITC |
| | | RASQDIGSELHWYQQKPDQP |
| | | PKLLIKYASHSTSGVPSRFS |
| | | GSGSGTDFTLTINGLEAEDA |
| | | GTYYCHQTDSLPYTFGPGTK |
| | | VDIKR |
| D2E7 | SEQ ID | DIQMTQSPSSLSASVGDRVT |
| VL | NO.: 10 | ITCRASQGIRNYLAWYQQKP |
| | | GKAPKLLIYAASTLQSGVPS |
| | | RFSGSGSGTDFTLTISSLQP |
| | | EDVATYYCQRYNRAPYTFGQ |
| | | GTKVEIKR |
| LINKER | SEQ ID | GGSGGGGSG* |
| | NO.: 11 | |
| B6-17 VL | SEQ ID | EIVLTQSPDFQSVTPKEKVT |
| | NO.: 12 | ITCRASQDIGSELHWYQQKP |
| | | DQPPKLLIKYASHSTSGVPS |
| | | RFSGSGSGTDFTLTINGLEA |
| | | EDAGTYYCHQTDSLPYTFGP |
| | | GTKVDIKR |
| CL | SEQ ID | TVAAPSVFIFPPSDEQLKSG |
| | NO.: 13 | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

*Note that in some embodiments, the C-terminus includes a serine (i.e., the light chain linker amino acid is GGSGGGGSGS(SEQ ID NO: 18))

A stock of ABT-122 at 3.9 mg/mL protein concentration was prepared in 15 mM Histidine, 80 mg/mL Sucrose, 0.2 g/L Polysorbate 80, pH 5.25. This stock was diluted from an original 27.46 mg/mL stock. A sulfo-tag labeled goat-anti human IgG antibody was purchased from Meso Scale Discovery (Gaithersburg, Md.). MSD standard 96 well streptavidin plates, MSD block buffer and 4×MSD read buffer with surfactant were purchased from Meso Scale Discovery (Gaithersburg, Md.). Plate sealers and 96-well deep well plates (2 mL) were obtained from Corning (Corning, N.Y.). Tween 20 was purchased from Sigma-Aldrich Inc. Phosphate buffered saline (PBS), MilliQ water, and tris buffered saline with 0.05% Tween-20 were prepared in a media room. The wash buffer contained 0.05% Tween-20 in PBS. Blocking Buffer contained 3% MSD Block in PBS. Assay Buffer was prepared by diluting blocking buffer three-fold in tris buffered saline containing 0.05% Tween-20. The 2× read buffer was prepared by diluting MSD 4× read buffer with surfactant 1:1 with MilliQ water.

A Freedom EVO (Tecan, Research Triangle Park, NC) automated liquid handling device was used to dilute samples and to serially dilute standards in 96-well deep well plates. A VWR Microplate Shaker (Henry Troemner LLC, Thorofare, NJ) was used to incubate the MSD assay plates with shaking at 600 rpm at room temperature. An ELx405 Plate washer apparatus (BioTek Instruments, Winooski, Vt.) was used to wash the plates with five wash cycles. A MSD Sector Imager 6000 (Meso Scale Discovery, Gaithersburg, Md.) was used to read/detect the plates by electro-chemiluminescence. Standard curves were analyzed using four-parameter logistic fit and sample concentrations were calculated by XLfit4 software (Version 4.2.1 Build 16).

### Sampling and Storage of Serum Samples

Blood samples were collected at pre-dose on Day 1; 0.1, 4, 9, 12, 24, 48, 72, and 168 hours post-dose on Day 1; 24, 72, and 168 hours following dosing on Day 8; 24 and 168 hours post-dose on Day 15; and 0.1, 4, 9, 12, 24, 48, 72 and 168 hours following dosing on Days 22, 28, 35, 42, and 50. Samples 24 and 48 hours after dosing on Day 1, and Days 18 and 50 were not sent for analysis. Serum samples were stored at −80° C. until analysis was performed.

### Biotinylated Human TNFα Capture MSD Assay

The study serum samples were analyzed for ABT-122 serum concentration using an MSD assay employing biotinylated human TNFα and sulfo-tag labeled goat anti human IgG antibody. The assay procedure involved washing MSD standard streptavidin plates five times with wash buffer. The plates were then blocked at room temperature with blocking buffer (150 μL/well) for one hour with shaking (600 rpm). High, medium, and low control samples (150 μg/mL, 20 μg/mL, and 1.5 μg/mL of ABT-122 spiked into cyno serum respectively) were prepared in batches, aliquoted for individual plate use, and stored at 80° C. Standard curve samples with a starting concentration of 175 μg/mL were prepared in cynomolgus monkey serum, aliquoted and stored at −80° C. until use.

Just prior to analysis, cynomolgus monkey serum samples were thawed at 4° C., mixed gently, and centrifuged at 14,000 rpm for two minutes at 24° C. in an eppendorf centrifuge. All samples were pre-diluted ten-fold in serum. All samples and both assay buffers (with and without 1% serum) were kept on ice prior to use. A Tecan Evo automated liquid handling station was utilized to dilute the samples. All standards, samples, and controls were prepared as duplicates in deep well plates by an initial one hundred fold (100×) dilution in assay buffer, followed by an additional seven-fold dilution in assay buffer containing 1% cyno serum. The standard curve covered a concentration range from 0.34 ng/mL to 250 ng/mL.

Diluted study samples, standard curve samples, control samples and blanks were added to a 2 mL deep well 96-well plate in duplicate (70 μL/well). Amounts of 0.1 μg/mL biotinylated TNFα (70 μL) and 1 μg/mL sulfo-tag labeled goat anti-human antibody (70 μL) were added to each well. The plates were covered, shaken for 20 seconds (600 rpm), and incubated at room temperature for one hour. The blocked MSD plates were washed and the samples, standards, and controls were transferred from the deep well plate to the MSD plate (75 μL/well). The MSD plates were incubated for one hour at room temperature with shaking (600 rpm). After incubation, the MSD plates were washed. Detection was performed by adding 150 μL/well 2× read buffer to the plate for 10 minutes. The chemiluminescence of each sample was detected using a MSD sector Imager 6000 system.

### Quantitation

Standard curve fitting and data evaluation were performed using XLfit4 software. A calibration curve was plotted from MSD luminescence signal versus theoretical standard concentrations. A four-parameter logistic model was used for curve fitting. The regression equation for the calibration curve was then used to back calculate the measured concentrations. Plates were deemed to have passed performance criteria if at least ⅔ of the quality controls (QCs) were within 30% of the expected values. The lower limit of quantitation (LLOQ), based on the 7000× dilution factor, was 2.38 μg/mL. Values below the quantitation limit (BQL) were omitted from calculations.

Pharmacokinetic parameters were calculated using WinNonlin software Version 5.0.1 (Pharsight Corporation, Mountain View, Calif.) by non-compartmental analysis with the NCA Model 201 and linear trapezoidal method. For calculations in WinNonlin, the time of dosing was defined as

Day 0, Time 0 and the graphs are depicted accordingly.

### Results and Discussion

The main pharmacokinetic parameters and trough concentrations of ABT-122 are summarized in Tables 4 and Table 5, respectively. The serum concentration-time profile observed after once a week dosing is depicted in FIG. 1. The serum concentration of ABT-122 from an individual animal subject is presented in Table 6. The raw data for standard curve back calculations and controls are presented in Table 7 and Table 8, respectively. Following repeat administration at 45 mg/kg IV, serum trough concentrations were reduced and AUC₀₋₁₆₈ₕᵣ measured after the fourth dose was lower compared to that measured after the first dose. The reduced test article concentrations following repeat dosing may be due to ADA formation.

TABLE 4
Main Pharmacokinetic Parameters of ABT-122 Following 45 mg/kg Weekly
Intravenous Administration for Four Weeks in Female Cynomolgus Monkey

| | | Cₘₐₓ | Cₘₐₓ/Dose | AUC_{0-168 hr} | AUC_{0-168 hr}/Dose |
|---|---|---|---|---|---|
| Animal | Dose # | (mg/mL) | (mg/mL/mg/kg) | (hr · mg/mL) | (hr · mg/mL/mg/kg) |
| 1 | 1^{st} | 1.60 | 0.036 | 162 | 3.6 |
| | 4^{th} | 1.83 | 0.041 | 98 | 2.2 |

TABLE 5
Serum C_{trough} Concentrations of ABT-122 Following
45 mg/kg Weekly Intravenous Administration for
Four Weeks in Female Cynomolgus Monkey

| C_{trough} (μg/mL) | | | | C_{trough}/Dose (μg/mL/mg/kg) | | | |
|---|---|---|---|---|---|---|---|
| 168 | 336 | 504 | 1008 | 168 | 336 | 504 | 1008 |
| hr | hr | hr | hr | hr | hr | hr | hr |
| 644.1 | 100.7 | 49.3 | BQL | 14.3 | 2.2 | 1.1 | N/a |

TABLE 6
ABT-122 Serum Concentrations Following 45 mg/kg IV Dosing
Once Weekly for Four Consecutive Weeks in Cynomolgus Monkey

| Time | ABT-122 Concentration | |
|---|---|---|
| (hrs) | (μg/mL) | Injection |
| 0 | 5.30 | 1 |
| 0.1 | 1599.81 | 1 |
| 4 | 1442.83 | 1 |
| 9 | 1135.70 | 1 |
| 12 | 1178.96 | 1 |
| 72 | 1021.95 | 1 |
| 168 | 644.07 | 1 |
| 192 | 562.50 | 2 |
| 240 | 726.32 | 2 |
| 336 | 100.72 | 2 |
| 360 | 1190.69 | 3 |
| 504 | 49.33 | 3 |
| 504.1 | 1832.63 | 4 |
| 508 | 1799.42 | 4 |
| 513 | 1367.18 | 4 |
| 516 | 1327.61 | 4 |
| 528 | 1063.35 | 4 |
| 552 | 709.89 | 4 |
| 672 | 13.00 | 4 |
| 840 | 7.24 | 4 |
| 1008 | BQL | 4 |

BQL: Below quantitation limit

TABLE 7
Summary Of ABT-122 Standard Samples

| | Theoretical Std. Conc. | | | | | | | | | | | | | |
|---|---|---|---|---|---|---|---|---|---|---|---|---|---|---|
| | 250 ng/mL | | 83.33 ng/mL | | 27.78 ng/mL | | 9.26 ng/mL | | 3.09 ng/mL | | 1.03 ng/mL | | 0.34 ng/mL | |
| Plate | Measured | % | Measured | % | Measured | % | Measured | % | Measured | % | Measured | % | Measured | % |
| # | Conc. | Diff. | Conc. | Diff. | Conc. | Diff. | Conc. | Diff. | Conc. | Diff. | Conc. | Diff. | Conc. | Diff. |
| 1 | 238.40 | −4.6 | 83.48 | 0.2 | 27.59 | −0.7 | 8.96 | −3.2 | 2.95 | −4.4 | 1.04 | 0.9 | 0.32 | −5.3 |
| | 255.05 | 2.0 | 88.01 | 5.6 | 27.91 | 0.5 | 9.13 | −1.4 | 3.26 | 5.7 | 1.06 | 2.8 | 0.36 | 5.1 |

TABLE 8
Summary Of ABT-122 Control Samples

| | | Theoretical Control Conc. | | | | | |
|---|---|---|---|---|---|---|---|
| | | 150 μg/mL | | 20 μg/mL | | 1.5 μg/mL | |
| Assay | Plate | Measured | % | Measured | % | Measured | % |
| Date | # | Conc. | Diff. | Conc. | Diff. | Conc. | Diff. |
| | 1 | 139.27 | −7.2 | 22.30 | 11.5 | 1.47 | −1.9 |
| | | 132.52 | −11.7 | 21.05 | 5.2 | 4.19 | 179.5 |

The safety and tolerability profile of ABT-122 was further analyzed by intravenous (IV) or subcutaneous (SC) injection of ABT-122 into cynomolgus monkeys. The subjects were administered different dosages (5, 45, 60, or 200 mg/kg/week) and various groups were administered dosages per week. Pharmacokinetics data are shown in Table 9 below.

TABLE 9
Summary Of TK/Tolerability Studies
Using ABT-122 in Cynomolgus Monkeys

| | ABT-122 Dosage | Mean Ctrough | | Mean AUC | |
|---|---|---|---|---|---|
| | (mg/kg/wk) | (μg/ml) | | (mg · hr/mL) | |
| Route | Dose | 1 | 4 | 1 | 4 |
| IV | 5 | 42 | — | 12.1 | — |
| IV | 45 | 644 | 13 | 162 | 98 |
| IV | 60 | 300 | 330 | 104 | 127 |
| IV | 100 | 580 | 1680 | 180 | 370 |
| IV | 200 | 1270 | 2690 | 380 | 630 |
| IV | 100 × 2 | 1320 | 1780 | 70 | 190 |
| IV | 200 × 2 | 3520 | 4600 | 200 | 480 |
| SC | 20 | 90 | BQL | 20 | na |
| SC | 60 | 240 | 320/BQL | 50 | 77/na |

### Example 2

### Product Metabolism and Pharmacokinetics of ABT-122 after Repeated Doses in Animals

In a 13-week Good Laboratory Practice (GLP) toxicity study, the PK parameters after repeated doses of ABT-122 DVD-Ig™ binding protein were assessed in groups of cynomolgus monkeys. Groups of male and female animals received once weekly IV doses (at 0, 20, 60, or 200 mg/kg/week) of the binding protein for a total of 14 doses. Additional groups of monkeys received a once weekly SC dose at 200 mg/kg/week over the same time interval. The data from this study indicated that AUC and Cmax values increased in a dose-related fashion. The harmonic mean of the terminal half-life calculated for recovery animals in the 200 mg/kg IV and SC groups was 8.0 days. Serum concentrations and AUC values for ABT-122 generally did not exhibit any sex-specific differences. The toxicokinetic parameters for females and males combined are presented in Table 10. In a separate 26-week GLP toxicity study in cynomolgus monkeys, toxicokinetic parameters from 60 and 200 mg/kg/week IV and 200 mg/kg/week SC dose groups were comparable to those observed in the 13-week study.

TABLE 10
ABT-122 Toxicokinetic Parameters After IV and SC Injection in
Cynomolgus Monkey Following 3 Months of Once Weekly Dosing

| | ABT-122 Dose (mg/kg/week) | | | |
|---|---|---|---|---|
| Toxicokinetic Parameter | 20 IV* | 60 IV | 200 IV | 200 SC |

Day 1

| | | | | |
|---|---|---|---|---|
| Number animals/group | 8* | 8 | 12 | 12 |
| Cₘₐₓ (mg/mL) | 0.997 (0.211) | 1.98 (0.40) | 8.55 (2.06) | 4.75 (0.53) |
| Cₘₐₓ/Dose (mg/mL/mg/kg) | 0.0499 (0.0105) | 0.0331 (0.0066) | 0.0428 (0.0103) | 0.0238 (0.0027) |
| AUC_{0-168 hr} (mg · hr/mL) | 50.3 (8.02) | 141 (33.4) | 549 (117) | 489 (53.9) |
| AUC_{0-168 hr}/D (mg · hr/mL/mg/kg) | 2.52 (0.40) | 2.35 (0.56) | 2.75 (0.58) | 2.45 (0.27) |
| Tₘₐₓ (hr) | NA | NA | NA | 38.0 (21.6) |

Day 50

| | | | | |
|---|---|---|---|---|
| Number animals/group | 2* | 8 | 12 | 12 |
| Cₘₐₓ (mg/mL) | 0.606 (0.016) | 2.43 (0.44) | 10.6 (5.5) | 4.74 (1.70) |
| Cₘₐₓ/Dose (mg/mL/mg/kg) | 0.0304 (0.00078) | 0.0405 (0.0074) | 0.0531 (0.0274) | 0.0237 (0.0085) |
| AUC_{0-168 hr} (mg · hr/mL) | 34.9 (12.1) | 172 (47.2) | 786 (393) | 525 (171) |
| AUC_{0-168 hr}/D (mg · hr/mL/mg/kg) | 1.74 (0.61) | 2.87 (0.78) | 3.93 (1.96) | 2.63 (0.86) |
| Tₘₐₓ (hr) | NA | NA | NA | 28.3 (13.2) |

Day 85

| | | | | |
|---|---|---|---|---|
| Number animals/group | NA | 8 | 12 | 12 |
| Cₘₐₓ (mg/mL) | NA | 2.45 (0.46) | 8.89 (3.38) | 4.85 (1.61) |
| Cₘₐₓ/Dose (mg/mL/mg/kg) | NA | 0.0409 (0.0076) | 0.0445 (0.0169) | 0.0243 (0.0080) |
| AUC_{0-168 hr} (mg · hr/mL) | NA | 177 (39.5) | 662 (335) | 540 (189) |
| AUC_{0-168 hr}/D (mg · hr/mL/mg/kg) | NA | 2.95 (0.66) | 3.31 (1.68) | 2.70 (0.95) |
| Tₘₐₓ (hr) | NA | NA | NA | 22.3 (5.8) |

Day 92

| | | | | |
|---|---|---|---|---|
| Number animals/group | NA | NA | 4 | 4 |
| Cₘₐₓ (mg/mL) | NA | NA | 9.71 (4.77) | 5.40 (2.09) |
| Cₘₐₓ/Dose (mg/mL/mg/kg) | NA | NA | 0.0486 (0.0238) | 0.0270 (0.0104) |
| AUC_{0-168 hr} (mg · hr/mL) | NA | NA | 651 (215) | 648 (277) |
| AUC_{0-168 hr}/D (mg · hr/mL/mg/kg) | NA | NA | 3.26 (1.08) | 3.24 (1.39) |
| Tₘₐₓ (hr) | NA | NA | NA | 30.0 (12.0) |

Recovery phase

| | | | | |
|---|---|---|---|---|
| Number animals/group | NA | NA | 1† | 4 |
| t_{1/2} | NA | NA | 8.3 | 7.9 (0.8) |

Data provided as mean (SD);
NA = not applicable
*On Day 50, only toxicokinetic parameters from Animals 2501 and 2504 are displayed, since all other animals within the 20 mg/kg dosegroup exhibited anti-ABT-122 ADA formation. Due to post-dose reactions (PDRs), a shortened administration scheme was applied to these animals and thus no toxicokinetic profiles after Day 50 are available.
†Three animals exhibited formation of ADAs and were excluded from half-life calculations.
§ Harmonic Mean ± Pseudo SD: 8.0 ± 0.8 days across both 200 mg/kg dosegroups.

ADAs measured in the 13-week cynomolgus GLP toxicity study indicate that the reductions in drug exposure were most likely indicative of ADA occurrence. In the 20 mg/kg/week IV dose group, the presence of ADAs in six of eight animals corresponded to decreased ABT-122 serum concentrations. In the higher dose groups, test item exposure was maintained during the dosing phase. Confirmed positive ADA titers were found during the recovery phase in three out of four animals in the 200 mg/kg/week IV dose group. ABT-122 induced ADA formation was not observed in any of the other treatment groups and exposure of the test item was maintained.

### Example 3

### ABT-122 Toxicology in Cynomolgus Monkeys

The safety profile of ABT-122 DVD-Ig™ binding protein was evaluated in two 13-week and one 26-week repeat-dose cynomolgus monkey toxicology studies, as well as in human and monkey tissue cross-reactivity studies. Intravenous and SC injection site tolerability was assessed during all GLP-compliant repeat-dose toxicology studies. Cynomolgus monkey was the only species utilized for toxicology studies due to insufficient cross reactivity of ABT-122 protein to both TNF-α and IL-17 from rodent, dog, and rabbit species.

No adverse toxicities were attributed to unintended pharmacology of ABT-122 among any of the repeat-dose toxicology studies, utilizing dose levels as high as 200 mg/kg via intravenous or subcutaneous administration.

Immune complex-mediated hypersensitivity responses were observed among some of the cynomolgus monkeys repeatedly administered weekly doses of ABT-122 via IV administration, only at 20 or 60 mg/kg, but were not observed with SC route of administration at any dose level. Hypersensitivity reactions are not an unexpected phenomenon for cynomolgus monkeys repeatedly administered humanized monoclonal antibodies.

Non-adverse test article-related findings were limited to minimal inflammation only at SC, but not IV, test article injection sites, and reduced size and numbers of splenic lymphoid follicles. These data were considered test article-related but non-adverse given the magnitude and reversibility of these changes. A disseminated fungal infection was observed in one animal at 60 mg/kg/week. The fungal infection may be related to the intended pharmacologic immunosuppressive properties of the test article. Among the over 90 animals treated with ABT-122, one animal treated with 200 mg/kg/week IV exhibited an idiosyncratic drug-induced immune-mediated thrombocytopenia that was resolving upon test article clearance during the recovery period.

No test item-related changes were observed among reproductive organs from sexually mature cynomolgus monkeys utilized during the GLP-compliant 13-week repeat dose toxicology study. Test article-treated (60 mg/kg IV, 200 mg/kg IV, 200 mg/kg SC) animals during the 26-week repeat dose toxicology study produced an anti-KLH IgM and IgG responses (T-cell dependent antibody responses) comparable to control animals. These data indicate that ABT-122-treated animals can produce an immune response to a neoantigen. A summary of all pivotal toxicology studies conducted with ABT-122 and a placebo local tolerability study are presented in Table 11.

TABLE 11
List of Toxicology Studies Conducted with ABT-122 DVD-Ig ™ Binding Protein

| Study | Species | Dosage/Route | Duration | NOAEL |
|---|---|---|---|---|
| Toxicology | Cynomolgus | 20, 60, 200/IV; | 13 weeks with 8-week | 200 mg/kg |
| | monkey | 200/SC; | recovery | once/week |
| | | mg/kg once/week | | |
| Repeat-Dose | Cynomolgus | 20 mg/kg | 13 weeks with 8-week | NA |
| Toxicokinetic | monkey | once/week; IV | recovery | |
| and Tolerability | | bolus, 2-hour | | |
| | | IV infusion or SC | | |
| Toxicology | Cynomolgus | 60, 200/IV; | 26 weeks with 15-week | Not |
| | monkey | 200/SC; | recovery | determined^{a,b} |
| | | mg/kg once/week | | |
| Tissue cross- | Human | NA | NA | NA |
| reactivity | | | | |
| Tissue cross- | Cynomolgus | NA | NA | NA |
| reactivity | monkey | | | |

IV = intravenous;
NA = not applicable;
NOAEL = no observed adverse effect level;
SC = subcutaneous
^{a}One 200 mg/kg IV monkey developed an idiosyncratic drug-induced immune-mediated thrombocytopenia (DITP) first detected at Day 113, which was resolving upon test article clearance during the recovery period.
^{b}One 60 mg/kg IV animal was euthanized on Day 205 due to complications associated with a disseminated fungal infection.

13-Week Toxicology Study of ABT-122 by IV and SC Injection in Cynomolgus Monkeys with an 8-Week Recovery Period

A 13-week GLP-compliant toxicity study was conducted in sexually mature cynomolgus monkeys at ABT-122 dose levels of 0 (placebo/vehicle; IV and SC), 20, 60, or 200 mg/kg once/week by IV injection (three to five minute bolus). The study also involved administering ABT-122 at 200 mg/kg once/week by SC injection (14 total doses/regimen). Dose levels for this study were selected based upon results from two four-week non-GLP studies which indicated that these doses would be well tolerated by cynomolgus monkeys, but dose levels at or below 60 mg/kg once/week could be immunogenic.

Designated subsets of animals during the 13-week GLP study were necropsied the day after Dose 14 (four/sex/group), or at the end of an eight-week recovery period (two/sex; control, 200 mg/kg IV, and 200 mg/kg SC groups only). Study parameters included clinical observations, injection site observations, body weight, food consumption, ophthalmologic and electrocardiologic examinations, clinical pathology (hematology, coagulation, clinical chemistry, and urinalysis), peripheral blood immunophenotyping, toxicokinetic and ADA analyses, gross necropsy, organ weight, routine histopathology, immunohistochemical evaluation of immune complex deposition in tissues, and electron microscopy evaluation of kidneys.

No adverse ABT-122 test article-related effects were observed among body weight, food consumption, injection site evaluations, ophthalmologic and electrocardiologic examinations, clinical pathology (hematology, coagulation, clinical chemistry, and urinalysis), peripheral blood immunophenotyping, gross necropsy, organ weight, and routine histopathology datasets for any individual animal at 20, 60, or 200 mg/kg that exhibited sustained serum ABT-122 levels during the dosing phase. No test item-related changes were observed in reproductive organs from these sexually mature animals.

Six of eight animals administered test article at 20 mg/kg IV exhibited PDRs consisting of shallow breathing, decreased activity, and/or retching/emesis after four to six doses. These were self-limited episodes with animals generally recovered within a few hours of dose administration, and were otherwise normal between weekly doses. With successive dose administrations the severity of PDR episodes often progressed and was not consistently ameliorated by diphenhydramine pretreatment. In one animal the progressive severity of the PDRs resulted in its moribund euthanasia following Dose 6 (Day 36). Test article administration was subsequently discontinued following Dose 7 or Dose 9 for all remaining 20 mg/k IV animals due to the prevalence and generally progressive severity of the observed PDRs (six of eight animals), the presumption that most animals were exhibiting exposure-altering ADA responses, and veterinary assessment of animal clinical signs.

Once dose administration was discontinued for animals at 20 mg/kg, the remaining 20 mg/kg animals were electively euthanized in healthy condition in order to conduct postmortem procedures proximal to the final dose administration and thus evaluate any observed histopathologic changes in PDR-affected animals relative to the concurrent non-PDR animals (only two of eight). No ABT-122 or PDR-related changes were observed among gross necropsy, organ weight, or routine histopathology (H&E) for any 20 mg/kg animal. Immunohistochemical (IHC) techniques revealed granular deposits containing human IgG (interpreted as ABT-122) and monkey immunoglobulins (interpreted as ADA) within neutrophils and macrophages of multiple tissues of 20 mg/kg monkeys with ADA/PDR. These IHC findings are consistent with an immune complex (ABT-122/ADA) association to the observed post-dose infusion reactions. Immune complexes were not observed via IHC in 20 mg/kg animals without PDR or in 200 mg/kg animals. No test article-related changes were observed among kidney glomeruli specimens examined by electron microscopy.

Test article exposures (AUC) were increased in a dose level-related fashion for animals that exhibited sustained serum ABT-122 levels during the dosing phase. Maximum exposures were achieved at 200 mg/kg IV, resulting in a Dose 13 (Day 85) AUC 0-168 of 662 mg·hr/mL. Only the individual 20 mg/kg animals with PDRs exhibited markedly decreased/absent serum ABT-122 concentrations with concurrent ADA.

One 60 mg/kg male exhibited slightly decreased body weight (approximately 11%) during Days 26 to 83 and was observed to have a mass in the mouth/throat that was cultured positive for streptococci and staphylococcus. Although these organisms are common incidental flora in cynomolgus monkeys based on experience at the testing facility, they contributed to the clinical condition of this particular animal. Concurrent throat cultures were not obtained from remaining study animals (including control) for comparison. The animal was given dietary treats, as well as antibiotics (Days 44 to 71). Body weight increased, the mass substantially resolved by Day 68 (data in clinical records), and the animal was otherwise unremarkable for the remainder of the study. Given the evident recovery of the animal following antibiotic treatment during test article administration, these findings are not considered adverse. Given the lack of comparable findings among other ABT-122-treated animals at this and higher dose levels, the observed clinical findings are of uncertain relationship to ABT-122 binding protein administration.

In conclusion, the NOAEL was 200 mg/kg based upon the lack of adverse test article-related effects in animals with sustained test article exposures at all dose levels/regimens. The PDR were not a direct effect of test article administration, but rather an indirect effect of the animal generating an antibody-dependent immune response to the humanized biologic test article.

13-Week Tolerability and Toxicokinetic Study Evaluating ABT-122 Administration by IV Bolus, IV Infusion, and SC Injection in Cynomolgus Monkeys with an 8-Week Recovery Period

In order to further characterize the PDRs observed during the preceding 13-week toxicology study, an additional 13-week non-GLP toxicity study was conducted in cynomolgus monkeys at the dose level of 20 mg/kg administered once/week to three cohorts of animals via either two-hour IV infusion, bolus IV injection (IV bolus), and SC injection (13 total doses/regimen) of ABT-122. All surviving animals were maintained for an eight-week recovery period following the last scheduled dose administration and were returned to the testing facility at the completion of the recovery period. Study parameters included clinical observations, body weight, food consumption, clinical pathology (hematology, coagulation, clinical chemistry, urinalysis), scheduled serum/plasma banking following test article and sham (vehicle only) dose administrations, toxicokinetic and ADA (titer, circulating immune complex [CIC] formation, isotyping, and epitope mapping) analyses, cytokine analyses, and complement activation analyses.

This study evaluated whether the severity and incidence of ADA-dependent hypersensitivity reactions using a 20 mg/kg dose (as observed in the first 13-week toxicology study) of ABT-122 could be influenced by dosing regimen (e.g., IV bolus, IV infusion, SC). The study was also designed to prospectively collect specific in vivo samples in order to better characterize the pathophysiology of any observed hyper sensitivity reactions; particularly the potential involvement of cytokines, complement, and circulating serum immune complex formation. No vehicle control group was included in this study as animals treated only with vehicle could not develop immunogenicity to ABT-122. However, each test article treated animal was given a sham (placebo) dose, and prospective in vivo samples were collected relative to this dose in order to ascertain procedural-dependent changes in any observed injection-dependent pathophysiologic endpoints.

No adverse ABT-122-related effects were observed among the individual animals in all dose regimens that exhibited sustained serum ABT-122 levels during the dosing phase. Two IV bolus animals were removed from continued dose administrations following repeated PDR during Doses 3 through 6 based upon the progressive severity of the post-dose clinical signs, unresponsiveness to prophylactic prednisolone administration, and veterinary assessment of animal clinical signs. One IV infusion animal was euthanized in moribund condition following Dose 13 due to the severity of the post-dose clinical signs. ADA and decreased serum test article exposures were evident in these animals relative to the observed PDR. The clinical observations for these animals were attributed to ADA-dependent hypersensitivity reactions. All other IV bolus animals were unremarkable during the dosing period, with no test article-related effects among study endpoints. No test article-related changes were observed among any animals in the SC dosing regimen.

Mean exposure profiles within each group were consistent with the route of administration among animals without detectable ADA. Individual animals in each 20 mg/kg dose regimen exhibited sustained ABT-122 exposures throughout the 13-week dosing period. No remarkable sex-specific differences were observed for toxicokinetic parameters among individual animals of a given dose route exhibiting sustained test article levels. No test article accumulation was observed following repeated dose administration.

However, multiple animals in each dosing regimen exhibited sustained ADA levels during the dosing phase without any evident PDRs. Importantly, no PDRs were observed among SC animals during the dosing phase despite this dose route producing the highest titer and highest incidence of ADA. The three animals with PDRs (i.e., animals described above) exhibited exposure-altering ADA throughout the majority of the dosing phase, consistent with an ADA-dependent hypersensitivity etiology for the observed post-dose clinical effects.

Additional analyses indicated that the ADAs were complexed with test article. The ADAs were determined to be IgG isotype (IgG subtypes not evaluated). No IgE or IgM (IgA reagents were cross-reactive to IgG) isotypes were identified among the ADA samples. Epitope competition assays indicate that the ADAs are directed against multiple epitopes of the test article, and were thus polyclonal. No particular ADA epitope phenotype was identified as specific/unique to animals with PDRs.

All animals with detectable ADA exhibited a marked increase in complement activation as measured by C3a generation and a corresponding decrease in 50% hemolytic complement (CH50) activity when administered ABT-122. All analytes returned to baseline/pre-dose ranges within 24 hours post-dose. These analyte changes did not differentiate between animals with or without PDR episodes. These changes were not observed for animals without ADA that were administered ABT—122. These changes were not_observed in any animal administered sham dose together indicating that both ADA and test article administration were required to produce the complement activation.

Further experiments were conducted to evaluate the relationship of cytokine profiles (IL-1Ra, IL-113, IL-2, IL-6, and IL-8) and histamine release relative to test article and sham (placebo) injections. However, no meaningful results were generated.

Other than findings described above, no treatment-related effects were observed among individual animals (all dose routes) for body weight, food consumption, or clinical pathology (hematology, coagulation, and clinical chemistry) evaluations.

The key results from this mechanistic ABT-122 study can be summarized as follows.

(1) Multiple animals in each dosing regimen (IV bolus, IV infusion, SC) exhibited sustained ADA levels without PDRs. (2) No PDRs were observed via the SC dose route, the route which produced the highest ADA titers. (3) None of the individual animals with ADA exhibited sustained serum ABT-122 exposures. (4) Three animals (two IV bolus and one IV infusion) exhibited PDRs characterized by ADA/ABT-122 complex-mediated complement activation. However, animals with ADA but no PDR also exhibited ADA/ABT-122 complexes and complement activation. (5) ADA responses were characterized as IgG isotype directed against multiple epitopes of ABT-122.

The data may indicate that the observed cynomolgus monkey PDRs are not a direct effect of test article administration, but rather an indirect effect of the animals generating an antibody-dependent immune response to the humanized biologic test article.

26-Week Toxicology Study of ABT-122 by IV and SC Injection in Cynomolgus Monkeys with a 15-Week Recovery Period

A 26-week cynomolgus monkey toxicology study was conducted in cynomolgus monkeys at dose levels of 0 (placebo/vehicle; via IV and SC administration), 60, or 200 mg/kg once/week IV bolus injection (60 minutes) and 200 mg/kg once/week SC injection (26 total doses/regimen) of ABT-122. Designated subsets of animals during the 26-week GLP study were to be necropsied the day after Dose 26 (4/sex/group), or at the end of a 15-week recovery period (2/sex; control, 200 mg/kg IV, and SC groups only). Study parameters included clinical observations, injection site observations, body weight, food consumption, ophthalmologic and electrocardiologic examinations, clinical pathology (hematology, coagulation, clinical chemistry, and urinalysis), peripheral blood immunophenotyping, toxicokinetic and ADA (titer, CIC formation, and isotyping) analyses, complement activation analyses, T-cell dependent antibody response, gross necropsy, organ weight, routine histopathology, and immunohistochemical evaluation of immune complex deposition in tissues.

One 60 mg/kg/week IV animal exhibited a PDR after Dose 5 and this animal was euthanized in moribund condition 2 days later based upon the severity of the observed post-dose reaction. The cause of moribundity for this animal was multisystemic hemorrhage and renal dysfunction secondary to immune complex (ABT-122/ADA) formation (observed in both serum and tissues). These effects were not a direct effect of test article administration, but rather an indirect effect of the animals generating an antibody-dependent immune response to the humanized biologic test article.

Administration of ABT-122 was associated with ADA-dependent decreases in serum test article exposures for six of eight animals (including the moribund euthanasia above) dosed at 60 mg/kg/week IV. The ADA was IgG isotype (not IgA, IgM, or IgE), was complexed with test article (ABT-122/ADA CIC), and correlated with acute complement activation. Dose administration was discontinued following Dose 16 for all surviving 60 mg/kg/week IV animals with detectable CIC, all of which were exhibiting a lack of sustained serum test article exposures during Weeks 3 to 10 and/or post-dose hypersensitivity reactions coincident with the CIC formation.

Other CIC-concurrent post-dose clinical signs among 60 mg/kg/week animals included lethargy and/or facial/generalized reddening. No ADA or post-dose reactions were observed during the dosing phase among animals given 200 mg/kg/week by IV or SC injection.

No sex-specific differences were observed among toxicokinetic parameters. Exposure (AUC and Cmax) values increased in a dose-related fashion among the IV infusion dose groups. The highest exposures were achieved at 200 mg/kg/week IV corresponding to a Day 176 (Dose 26) AUC0-166 of 616 mg·hr/mL.

One 60 mg/kg/week IV animal was euthanized on Day 205 due to complications associated with a disseminated fungal infection. The test article did not cause the fungal infection, but rather the intended pharmacology of the test article predisposed the animal to recrudescence of a latent fungal infection. No evidence of fungal infections was observed among remaining study animals during this study.

A single animal at 200 mg/kg/week IV experienced thrombocytopenia first detected on Day 113, attributable to an etiology of drug-induced immune thrombocytopenia (DITP). This animal had moderately decreased platelets of 105,000/μL on Day 113 that progressed to 10,000/μL on Day 127, concurrent with an increase in mean platelet volume and no change in mean platelet component. The initial decrease was associated with petechiae but normal prothrombin time and activated partial thromboplastin (APTT) clotting times. Dosing for this animal was discontinued following Dose 18 (Day 120) due to the low platelet count. With the exception of petechiae, this animal was clinically asymptomatic throughout its course.

During the recovery phase, the platelets remained in a range of 18,000-53,000/μL concurrent with detectable test article, although no petechiae were noted. The prolonged decrease in platelets was consistently associated with an increase in mean platelet volume (MPV), consistent with ongoing bone marrow platelet production, and normal mean platelet concentration (MPC), indicating normal activation of platelets. The test item was undetectable in serum as of Day 225. The platelet count increased to 107,000/μL and the mean platelet volume recovered by Day 231. Adequate numbers of megakaryocytes were observed microscopically in the bone marrow at necropsy on Day 232. Taken together, the constellation of clinical signs and laboratory abnormalities reported for this thrombocytopenia animal were strongly consistent with a DITP, which is an idiosyncratic drug—sensitivity reaction caused by drug-dependent antibodies that bind to platelets (Arnold et al. (2002) J. Thromb. Haemost. 11(1):169-76). DITP can be directly confirmed through laboratory testing to detect drug-dependent anti-platelet antibodies; however, validated assays are difficult to achieve and not routinely available or reliable (Heikal et al. (2013) Am. J. Hematol. 88(9):818-21).

Consequently, the diagnosis of DITP is frequently achieved by considering not only established clinical criteria but also the temporal relationship to drug administration and the absence of associated laboratory abnormalities that would indicate other pathologic mechanisms. In this animal, the onset of thrombocytopenia following multiple intravenous infusions of ABT-122, lack of platelet activation (as indicated by stable MPC levels and the absence of systemic sequelae of activation), evidence of effective thrombopoeisis (increased MPV and adequate megakaryocytes), adequate secondary coagulation (normal prothrombin time and APTT) and partial recovery of platelet counts following decrease of test article plasma exposure were strongly suggestive of DITP, and the absence of other tissue pathologies strongly support a DITP etiology. The absence of effects on RBC and WBC lineages either in the bone marrow or the periphery are also consistent with this etiology.

To date, over 90 cynomolgus monkeys have received multiple injections of ABT-122 at doses ranging from 20 to at least 200 mg/kg/week. Across all of the other cynomolgus monkey studies there have been no other observed platelet effects, including a variety of doses and durations of ABT-122 administration. The above described animal, administered test article via the IV route, was the only incidence of DITP. This observation is consistent with the occurrence of DITP and other types of idiosyncratic drug-sensitivity reactions, in which only a small number of subjects develop the disorder (Aster et al. (2007) N. Engl. J. Med. 357(6):580-587). No changes in platelet values have been observed in any other ABT-122 treated cynomolgus monkey across the various repeat dose toxicology studies.

Another test article, but non-adverse finding, in this 26-week study was a minimal to moderate reduction in the size and number of splenic lymphoid follicles observed among two to three animals in each test article-treated group as compared to control animals, with occasional loss of definition between the splenic follicular germinal centers and mantle zones. These findings were considered test article-related but non-adverse given the potential relationship to the intended pharmacologic immunosuppression properties of the test article and evidence of reversibility. In addition, there were no test article alterations in anti-KLH IgM or IgG antibody responses (T-cell dependent antibody responses; TDAR). All test article-treated animals produced an anti KLH IgM and IgG response after KLH immunization comparable to the control animals, indicating that ABT-122-treated animals can produce an immune response to a neoantigen. Minimal perivascular mononuclear cell infiltrates were observed at the final test article SC injection sites among 200 mg/kg/week SC animals. These findings were considered non-adverse given the small magnitude of the change and evidence of reversibility. There were no ABT-122-related changes among any other study parameter in the aforementioned animals, nor in any study parameter in the remaining test article-treated animals.

In conclusion, no toxicities were attributed to unintended pharmacology of ABT-122 administration. Non-adverse and reversible test article-related changes were limited to minimal inflammation only at SC (but not IV) test article injection sties, and reduced size and numbers of splenic lymphoid follicles observed among two to three individual animals in each test article dose group. A disseminated fungal infection in one animal at 60 mg/kg/week IV was attributed to pharmacologic immunosuppression of this animal resulting from test article administration. There were two types of immune-mediated effects observed in this study. ADA-dependent immune responses following repeated ABT-122 administration at 60 mg/kg/week IV (but not 200 mg/kg IV or 200 mg/kg/week SC) were associated with immune complex-mediated hypersensitivity events only at this 60 mg/kg/week IV dosage, consistent with an indirect effect of the animal generating an antibody-dependent immune response to the humanized biologic test article. One animal at 200 mg/kg/week IV exhibited a clinically asymptomatic episode of an idiosyncratic drug-induced immune-mediated thrombocytopenia after repeated test article exposure, which was resolving upon test article clearance during the recovery period.

### Tissue Cross-Reactivity

Tissue cross-reactivity studies were conducted using ABT-122 and cryo-preserved tissues from human and cynomolgus monkey species. When observed, the test article cross reactivity was restricted to the cytoplasm without evidence of membrane staining Test article cross-reactivity was evident in myocytes of intestinal smooth muscle and colon epithelium from both human and cynomolgus monkey tissues. Test article cross-reactivity was also observed in smooth myocytes from cynomolgus monkey cervix. These results indicate comparable staining among representative human and cynomolgus tissues, and a lack of evident membrane staining among immunoreactive tissue types. The lack of membrane staining is consistent with the pharmacologic binding of ABT-122 to soluble TNF and IL-17.

No local intolerance was observed at IV and/or SC injection sites during the GLP-compliant 13-week repeat-dose toxicology study. Minimal perivascular mononuclear cell infiltrates were observed at the final test article SC injection sites among 200 mg/kg/week SC animals during the 26-week repeat-dose toxicology study. These findings were considered non-adverse given the small magnitude of the change and evidence of reversibility.

### Example 4

### Study M12-704-ABT-122 Demonstrates Dual Binding of TNF and IL-17 In Vitro and Dual Functional Activity in Serum from Subjects in a Phase I Study

Levels of TNF and IL-17 protein are increased in rheumatoid synovial tissue compared to normal tissue. Superior therapeutic responses may be achieved in RA patients by neutralizing TNF and IL-17 simultaneously compared with neutralizing either cytokine alone. See Table 1.

This Example assessed whether ABT-122 in sera would bind and neutralize TNF and IL-17 in vitro. Accordingly, sera from subjects treated with a single dose of ABT-122 were analyzed in a Phase 1 study to determine whether the sera neutralized both cytokines.

Surface plasmon resonance (SPR) assessed the sequential binding of ABT-122 to human TNF and IL-17. Stoichiometry was calculated based on the respective molecular weights of the antigens and DVD-Ig protein, as well as their respective binding and capture levels expressed in resonance units according to the manufacturer's guidelines (See Biacore Inc.).

Functional activity of ABT-122 was determined using an in vitro assay of human fibroblast-like synoviocytes (FLS) derived from rheumatoid arthritis patients. The FLS were stimulated with recombinant human TNF and IL-17 resulting in a synergistic production of IL-6. Inhibition of the IL-6 response was evaluated using either recombinant ABT-122 or with serum samples from healthy volunteers who had received ABT-122 as a single intravenous (IV) infusion (0.1 mg/kg-10 mg/kg) or subcutaneous (SC) administration (0.3 mg/kg-3.0 mg/kg) in a Phase I study.

Human FLS secrete IL-6 in response to TNFα and IL-17 stimulation. Human FLS (Cell Applications Inc., catalogue number 408RA-05a, donor 1982) cells were maintained in Synoviocyte Growth Media (Cell Applications Inc., catalogue number 415-500). Cells were grown the day of the assay in T150 flasks until the cells were about 70-80% confluent. The assay media used included RPMI media (Invitrogen Inc., catalogue number 11875-093) with 10% human serum (Sigma Inc., catalogue number H3667, lot number SLBF2562V), 2 mM L-glutamine (Invitrogen Inc., catalogue number 25030-081, and 1% P/S (Invitrogen Inc., catalogue number 15140-122). The FLS were trypsinized and placed in assay media at a concentration of 2.5×10⁵ cells/mL. A volume (50 μL) of the FLS in assay media was transferred into each well of a 96-well flat-bottom tissue culture plate (Costar Inc., product number 3599). Subject serum was serially diluted into RPMI and human serum for a final concentration of 10% human serum. Subject serum dilutions were calculated from the pK data provided.

Human IL-17his protein (product A1174832.0, Lot#1746670) was diluted to 8 ng/mL (four fold; 4×) in assay media. Human TNF-α protein (product A869094, Lot#1276829) was diluted to 0.8 ng/mL (4×) in assay media. A volume (65 μl) of diluted subject serum was incubated with 65 μl of IL-17 antigen and TNF-α antigen for one hour at room temperature. Control FLS samples (lacking ABT-122) were incubated with either: a combination of TNF-α and IL-17, TNF alone, IL-17 alone, or neither TNF-α or IL-17. A volume (fifty microliters) of this serum/IL-17/TNF mixture was added to each well of plated human FLS cells. In each well the final concentration of IL-17his protein was 2 ng/ml (60 pM). The final concentration of TNF-α protein was 0.2 ng/mL (3.84 pM). Cells were incubated at 37° C. for about 24 hours. The media supernatants were then collected and analyzed for IL-6 expression using a commercial Meso Scale Discovery kit (catalogue K112AKA-4) according to the manufacturer's instruction. IC50 values were obtained using logarithm of antibody versus IL-6 amount variable slope fit.

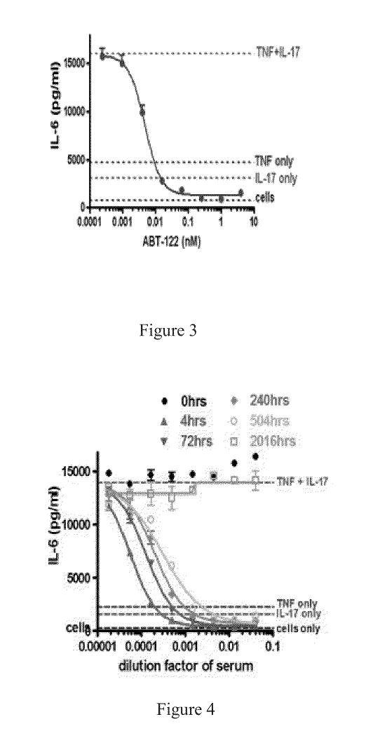

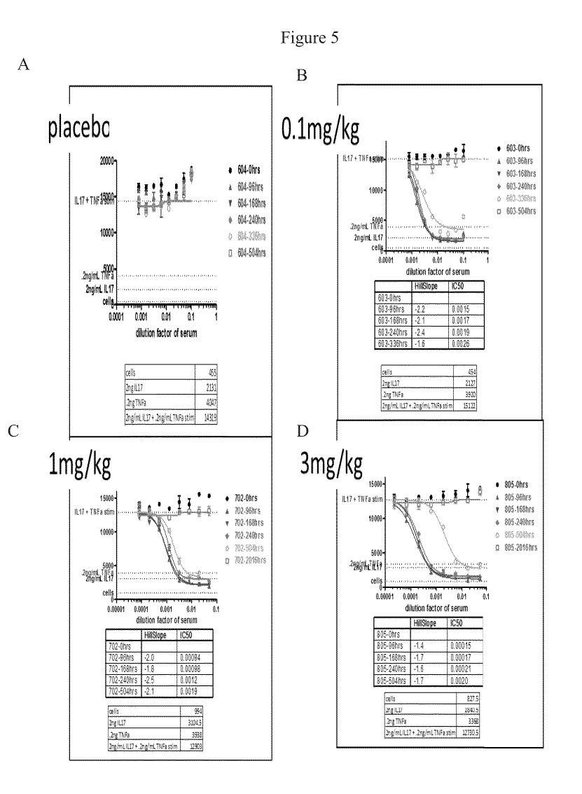

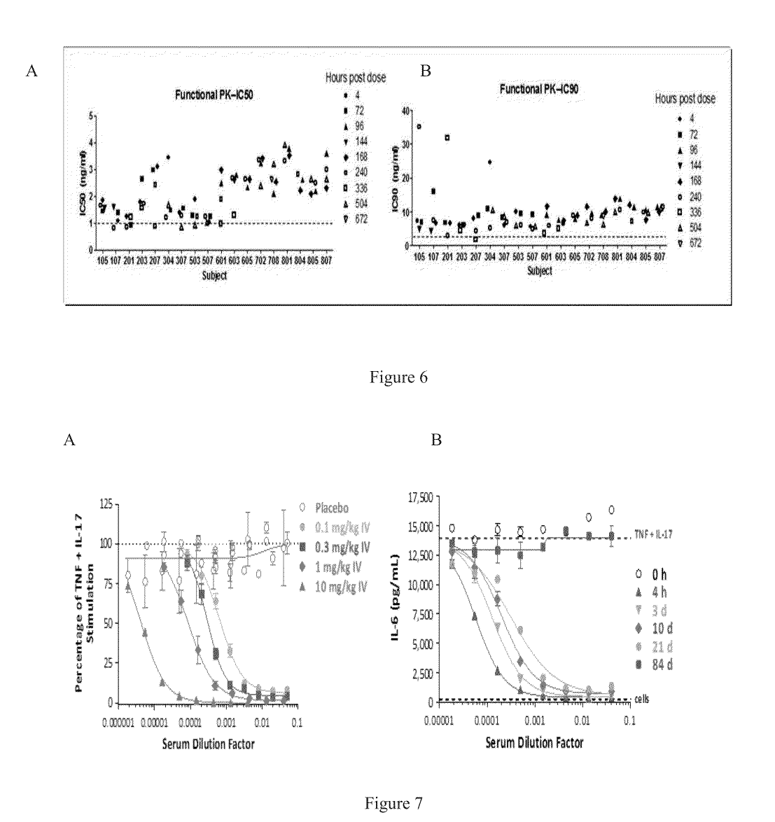

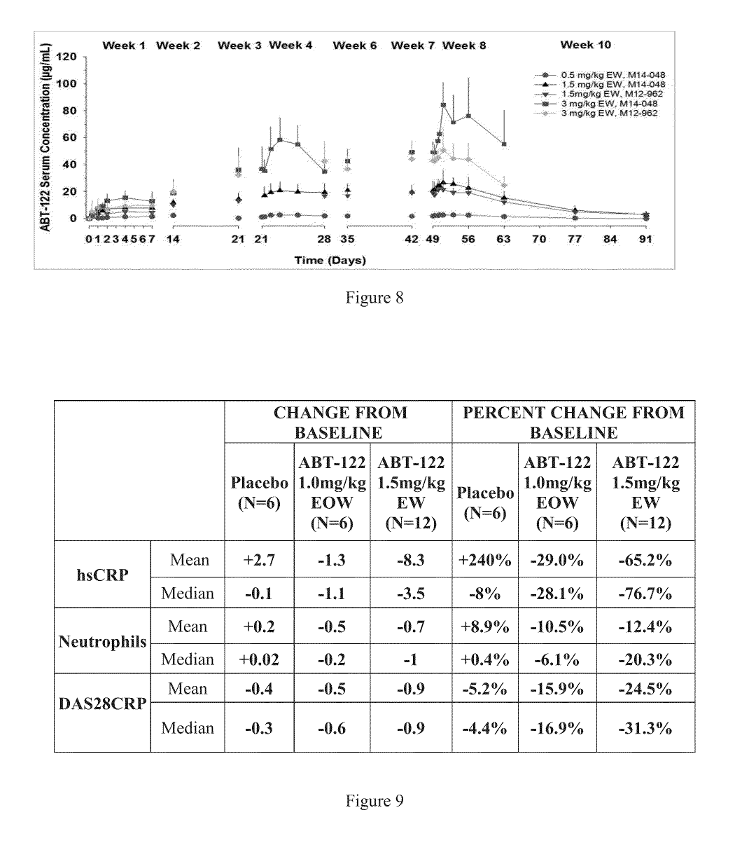

ABT-122 simultaneously bound to TNF and IL-17, and data showed binding of a similar amount of TNF per ABT-122 molecule regardless of the occupancy of the IL-17 binding sites and vice versa. From the SPR sensogram data it was observed that the binding kinetics of one antigen binding domain was not significantly affected by the presence of the second antigen at the other binding domain. Irrespective of whether or not IL-17 is saturated, data show that the TNF signals (measured in resonance units; RU), were 43 RU and 45 RU. The IL-17 SPR values were 56 RU and 61 RU. In the functional assay, only ABT-122 fully neutralized the synergistically-induced IL-6 release from FLS stimulated by the combination of TNF and IL-17, whereas individual monoclonal antibodies against either cytokine only partially neutralized the IL-6 production. Sera from subjects receiving ABT-122 demonstrated comparable potency (IC₅₀ and IC₉₀) to that defined by ABT-122 in the assay and was consistent across dose groups. See FIGS. 3-6. Data from FIG. 3 indicate that ABT-122 had an IC₅₀ of 1 ng/mL and an IC₉₀ of 3.4 ng/mL. The neutralization results for ABT-122 were improved compared to results observed for both adalimumab, and the IL-17 antibody of ABT-122 alone. Furthermore, serum samples from healthy subjects were able to block TNF/IL-17-induced IL-6 release in a dose-dependent manner (FIG. 7 panel A). Data show that ABT-122 has a dose-dependent pharmacologic activity and engaged both serum TNF and IL-17 targets in healthy subjects three weeks after a single dose of ABT-122 (FIG. 7 panel B). Full neutralization was time- and dose-dependent through 21 days, and up to 10 mg/kg, respectively.

Thus, ABT-122 was effective for dual targeting of TNF and IL-17. Data show that ABT-122 simultaneously bound to and neutralized TNF and IL-17 in vitro. The dual cytokine neutralization by ABT-122 in sera from healthy subjects was also confirmed in an ex vivo assay where fully functional levels of this ABT-122 were maintained for up to three weeks after a single dose. The dose range of ABT-122 in this phase 1 study was appropriate for continuing to further characterize the clinical profile of dual TNF/IL-17 inhibition in RA and other inflammatory diseases.

Data herein demonstrate that ABT-122 simultaneously bound to and neutralized TNF-α and IL-17 in vitro. Furthermore, functional levels of ABT-122 are maintained for up to three weeks in healthy subjects. Examples herein also show that ABT-122 demonstrated an acceptable safety profile following single dose administration up to 3 mg/kg SC and 10 mg/kg IV.

### Study M12-704 Involving Pharmacokinetics and Product Metabolism of ABT-122 in Humans

ABT-122 DVD-Ig™ binding protein has been evaluated in four Phase 1 studies where study conduct is complete and in two ongoing Phase 2 studies. Final PK data are available for the First in Human (FIH) Study M12-704 and Study M14-346. Preliminary pharmacokinetic data are available for Phase 1 Studies M14-048 and for the first 3 dose groups in the ongoing Phase 1 Study M1-962. See Table 12.

In Study 1 (Study M12-704), single-ascending IV doses (0.1, 0.3, 1, 3 and 10 mg/kg) and SC doses (0.3, 1 and 3 mg/kg) doses were administered and evaluated in healthy volunteers. In Studies 2 and 3 (Study M12-962 and M14-048; and M12-963 respectively) multiple ascending SC doses (1 mg/kg biweekly, 0.5 to 3 mg/kg weekly for 8 weeks) were evaluated in subjects with rheumatoid arthritis on stable methotrexate (MTX) therapy. ABT-122 pharmacokinetic parameters were estimated using non-compartmental analyses.

TABLE 12
ABT-122 DVD-Ig ™ Binding Protein Clinical Studies

| | | Number of Subjects | | | |
|---|---|---|---|---|---|
| Protocol | Phase | (Actual/Planned)^{a} | Study Design | Primary Objective | Status |
| M12-704 | 1 | 64/64 | Single-ascending dose, placebo-controlled, | Safety, tolerability, PK, and | Completed; |
| | | | double-blind (DB), randomized study to | immunogenicity of ABT-122 with | clinical study |
| | | | assess the safety, tolerability, PK and | a single dose IV infusion or SC | report complete. |
| | | | immunogenicity of single IV infusion and | injection. | |
| | | | single SC injection of ABT-122 in healthy | | |
| | | | subjects. | | |
| M12-962 | 1 | 25/24^{b} | Randomized, DB, placebo-controlled multiple | Safety, tolerability and PK | Ongoing; |
| | | | dose study with 4 parallel treatment groups to | following multiple SC injections | preliminary data |
| | | | assess the safety, tolerability, PK and | of ABT-122 in subjects with RA. | are available for |
| | | | immunogenicity of varying doses of ABT-122 | | the 3 groups of |
| | | | given with MTX to subjects with RA. | | subjects with RA. |
| M14-048 | 1 | 19/24 | Randomized, DB, placebo-controlled multiple | Safety, tolerability and PK | |
| | | | dose study to assess the safety, tolerability and | following multiple SC injections | Completed; |
| | | | PK of multiple SC injections of ABT-122 in | of ABT-122 in subjects with RA. | clinical study |
| | | | subjects with RA. | | report ongoing. |
| M14-346 | 1 | 24/24 | Single SC dose, open-label, parallel group, | Bioavailability, safety and | Completed; |
| | | | randomized, single-center study to evaluate | tolerability of a 100 mg/mL | clinical study |
| | | | the bioavailability of a high concentration | concentration formulation of | report ongoing. |
| | | | formulation compared to the current low | ABT-122 compared to the current | |
| | | | concentration formulation of ABT-122 in | 50 mg/mL concentration | |
| | | | healthy subjects. | formulation of ABT-122. | |
| M12-963 | 2 | 12/160 | Randomized, DB, double-dummy, | Estimation of ABT-122 treatment | Ongoing |
| | | | active-controlled, parallel-group, multicenter | assessed by American College of | |
| | | | study of ABT-122 given with MTX in subjects | Rheumatology response rates | |
| | | | with active RA who have an inadequate | (ACR20) at Week 12, safety and | |
| | | | response to MTX | tolerability of ABT-122 in | |
| | | | | subjects with RA | |
| M12-965 | 2 | 0/80 | 24-week open-label extension in subjects who | Assess the long-term safety and | Ongoing |
| | | | have completed Study M12-963 | tolerability of ABT-122 in | |
| | | | | subjects with RA | |

US = United States
PK = pharmacokinetics;
IV = intravenous;
SC = subcutaneous;
RA = rheumatoid arthritis;
EU = European Union;
MTX = methotrexate
^{a}Enrollment as of 1 Jan. 2015.
^{b}One subject in the placebo group withdrew consent and, in accordance with the protocol, was replaced following agreement between the investigator and AbbVie. This accounts for 25 subjects enrolled versus the projected enrollment of 24 subjects.

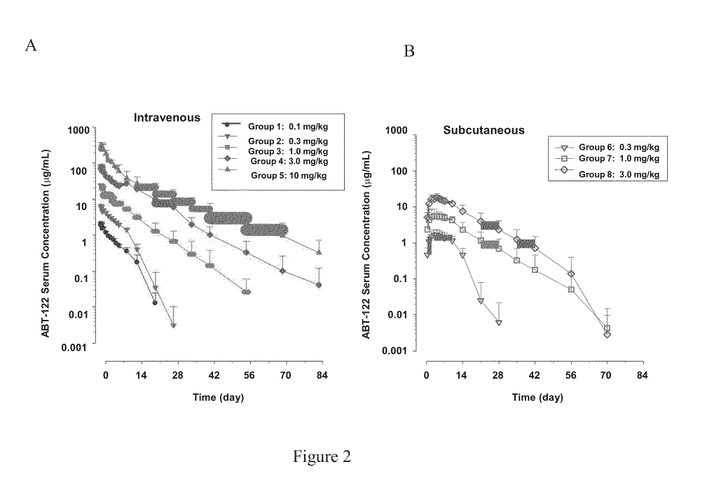

Study M12-704, a randomized, DB, placebo-controlled study in healthy adult subjects to assess the safety, tolerability, and PK of escalating, single doses (administered IV as a continuous 2-hour infusion or SC) of ABT-122, was conducted in 2 parts (see Table 13 panel A and Table 13 panel B). Part 1 consisted of 5 IV dose groups (0.1, 0.3, 1, 3, and 10 mg/kg), and Part 2 consisted of 3 SC dose groups (0.3, 1, and 3 mg/kg). The mean±SD serum concentration-time profiles for the PK parameters from Study M12-704, following a single IV or SC dose of ABT-122, are presented in FIG. 2 panel A and FIG. 2 panel B on a log-linear scale (N=6 subjects per dose group). The mean and standard deviation (SD) values for the PK parameters of ABT-122 after a single dose are shown in Table 13 below. Pharmacokinetic data were available from 48 healthy and 31 RA subjects with median body weights of 78 (range; 52-98) and 79 (range; 47-111) kg, respectively. ABT-122 pharmacokinetic profile showed multi-exponential disposition with more than dose-proportional exposure at the lower doses (0.1-1 mg/kg) and approximately dose-proportional exposure at doses >1 mg/kg. With SC dosing, ABT-122 absolute bioavailability was approximately 50% and maximum serum concentrations were reached 3-4 days after dosing. After the last dose, ABT-122 AUC accumulation ratio was 3.8 to 5.6 with steady-state appearing to be achieved by 5 weeks of SC dosing. ABT 122 Cmax to Ctrough ratio was 2.6 and 1.3 for biweekly and weekly dosing, respectively.

TABLE 13A
Dose Groups in Parts 1 And Part 2 of Study M12-704

| | | | Number of |
|---|---|---|---|
| | Group | Regimen | Subjects |
| Part I | Group 1 | 0.1 mg/kg ABT-122 IV or placebo | 8 (6:2) |
| | Group 2 | 0.3 mg/kg ABT-122 IV or placebo | 8 (6:2) |
| | Group 3 | 1 mg/kg ABT-122 IV or placebo | 8 (6:2) |
| | Group 4 | 3 mg/kg ABT-122 IV or placebo | 8 (6:2) |
| | Group 5 | 10 mg/kg ABT-122 IV or placebo | 8 (6:2) |
| Part II | Group 6 | 0.3 mg/kg ABT-122 SC or placebo | 8 (6:2) |
| | Group 7 | 1 mg/kg ABT-122 SC or placebo | 8 (6:2) |
| | Group 8 | 3 mg/kg ABT-122 SC or placebo | 8 (6:2) |

TABLE 13B
Pharmacokinetic Parameters Of ABT-122 Following
A Single Dose Of ABT-122 (Mean ± SD)

| | | | | | |
|---|---|---|---|---|---|
| Pharmacokinetic | Group 1 | Group 2 | Group 3 | Group 4 | Group 5 |
| Parameter (Units) | 0.1 mg/kg | 0.3 mg/kg | 1.0 mg/kg | 3.0 mg/kg | 10 mg/kg |

Intravenous Doses

| N | 6 | 6 | 6 | 6 | 6 |
|---|---|---|---|---|---|
| Tₘₐₓ (hr) | 4.3 ± 0.8 | 5.0 ± 2.7 | 5.0 ± 2.8 | 4.7 ± 1.0 | 6.1 ± 2.2 |
| Cₘₐₓ (μg/mL) | 2.4 ± 0.3 | 7.5 ± 1.0 | 27.2 ± 2.5 | 81.5 ± 19.1 | 348 ± 92.7 |
| AUC_{∞} (μg · hr/mL) | 276 ± 35.3 | 931 ± 95 | 4016 ± 524 | 16067 ± 3624 | 43733 ± 9741 |
| t_{1/2}^{a} (hr) | 85.3 ± 26.5 | 69.0 ± 21.5 | 124 ± 37.1 | 151 ± 48.5 | 93.9 ± 24.1 |
| | [3.6 days] | [2.9 days] | [5.2 days] | [6.3 days] | [3.9 days] |
| Cₘₐₓ/Dose | 23.7 ± 2.7 | 25.0 ± 3.3 | 27.2 ± 2.5 | 27.2 ± 6.4 | 34.8 ± 9.3 |
| (μg/mL)/(mg/kg) | | | | | |
| AUC_{∞/}Dose | 2759 ± 353 | 3102 ± 317 | 4016 ± 524 | 5356 ± 1208 | 4373 ± 974 |
| (μg · hr/mL)/(mg/kg) | | | | | |
| | | Group 6 | Group 7 | Group 8 | |
| | | 0.3 mg/kg | 1.0 mg/kg | 3.0 mg/kg | |

Subcutaneous Doses

| | | | |
|---|---|---|---|
| N | 6 | 6 | 6 |
| Tₘₐₓ (hr) | 76 ± 18.1 | 100 ± 46.6 | 92 ± 9.8 |
| | [3.2 days] | [4.2 days] | [3.8 days] |
| Cₘₐₓ (μg/mL) | 1.7 ± 0.7 | 6.1 ± 3.1 | 18.3 ± 4.7 |
| AUC∞ (μg · hr/mL) | 496 ± 130 | 2138 ± 690 | 6659 ± 1803 |
| t_{1/2}^{a} (hr) | 88.1 ± 59.0 | 75.3 ± 123 | 75.6 ± 78.3 |
| | [3.7 days] | [3.1 days] | [3.2 days] |
| Cₘₐₓ/Dose | 5.8 ± 2.2 | 6.1 ± 3.1 | 6.1 ± 1.6 |
| (μg/mL)/(mg/kg) | | | |
| AUC_{∞}/Dose | 1652 ± 433 | 2138 ± 690 | 2220 ± 601 |
| (μg · hr/mL)/(mg/kg) | | | |

Note:
Values in days presented in [ ] were calculated using the average or harmonic mean value/24.
^{a}Harmonic mean and pseudo standard deviation.

Following a single dose of ABT-122, mean Cmax and AUC at the highest dose of 10 mg/kg IV were 348 μg/mL and 43,733 μg·hr/mL, respectively. ABT-122 harmonic mean half-lives ranged from 2.9 to 6.3 days following IV administration, with longer half-lives observed following the 1.0 and 3.0 mg/kg doses. Following SC administration, mean half-lives ranged from 3.1 days to 3.7 days. Concentrations appear to be sustained near Cmax for approximately 7 days after dosing. The estimated relative bioavailability after SC administration was 48%. The PK (Cmax and AUC) of ABT-122 appears to be more than dose proportional following 0.3 mg/kg to 10 mg/kg single IV doses and dose proportional following 0.3 mg/kg to 3 mg/kg single SC doses.

The presence of ADA was measured with a validated immunoassay. Sampling for ADA occurred prior to ABT-122 dosing (pre-dose) and following the single dose of ABT-122 on days 15, 22, 29, 36, 43, 57, 71 and 85. There was a high incidence of detectable ADA titer after ABT-122 administration in all dose groups. However, in the majority of subjects, ABT-122 exposure was achieved in the presence of ADA. None of the subjects in the placebo IV or SC groups had a positive ADA titer at any time during the study. Most subjects had detectable titer at the first time point post ABT-122 dosing (Study Day 15). One subject in Group 5 (10 mg/kg IV) had a high ADA titer (range: 49700-129000 titer units) and a decrease in ABT-122 serum concentration on Day 15. There was no difference in ADA titer incidence following IV versus SC administration, no clear correlation between ADA titer and dose administered, and no association with the safety or tolerability profile.

### Study M12-704 Involving Safety of ABT-122 in Humans

Study M12-704, a FIH, Phase 1, randomized, DB, placebo-controlled study in healthy adult subjects was performed to assess the safety, tolerability, and PK of escalating, single doses (administered IV as 2-hour infusion or SC) of ABT-122. The primary objective of this study was to determine safety, tolerability, PK, and immunogenicity of ABT-122 with a single dose IV infusion or SC injection. The completed study involved a total of 64 subjects. The study was conducted in 2 parts. Part 1 consisted of 5 IV sequential dose groups, and was followed by Part 2, which consisted of 3 SC sequential dose groups as presented in Table 13 panel A. If acceptable safety, tolerability, pharmacokinetics, and immunogenicity results were obtained from IV doses (i.e., Groups 1-5), then subjects were allowed to proceed to SC doses (i.e., Groups 6-8). Eight subjects in each group were randomized in a 6:2 ratio to receive ABT-122 or to receive a placebo. Subjects are male or female; 18-55 years old; in good general health; have a negative pregnancy test results for female; and no previous exposure to the biologic. Table 13 panel B compiles the pharmacokinetic parameters of ABT-122 following single doses of ABT-122 (Mean±SD).

ABT-122 harmonic mean half-lives (t_{1/2}) ranged from 2.9 to 6.3 days following IV administration. The estimated bioavailability after SC administration was 48%. The PK (Cₘₐₓ and AUC_{∞}) of ABT-122 appears to be more than dose proportional following 0.3 mg/kg to 10 mg/kg single IV doses and dose proportional following 0.3 mg/kg to 3 mg/kg single SC doses.

The presence of ADA was measured with a validated immunoassay. Sampling for ADA occurred prior to ABT-122 dosing (pre-dose) and following the single dose of ABT-122 on Days 15, 22, 29, 36, 43, 57, 71, and 85. There was a high incidence of detectable ADA titer after ABT-122 administration in all dose groups. However, in the majority of subjects, ABT-122 exposure was achieved in the presence of ADA. None of the subjects in the placebo IV or SC groups had a positive ADA titer at any time during the study.

Most subjects had detectable titer at the first time point post ABT-122 dosing (Study Day 15). One subject in Group 5 (10 mg/kg IV) had a high ADA titer (range: 49700-129000 titer units) and a decrease in ABT-122 serum concentration on Day 15. There was no difference in

ADA titer incidence following IV versus SC administration, no clear correlation between ADA titer and dose administered, and no association with the safety or tolerability profile.

### Part 1—IV Dosing

Twenty-one of the 30 subjects (21/30, 70.0%) who received ABT-122 IV reported 1 or more AEs compared to the 9 of 10 subjects (9/10, 90.0%) who received placebo IV. The most frequently reported AEs for subjects given ABT-122 included upper respiratory tract infection (5 subjects) and headache (3 subjects). The most frequently reported AEs for subjects given placebo included upper respiratory tract infection (3 subjects). The incidence of upper respiratory tract infections was higher in the placebo subjects. All other events were reported in ≦2 subjects each for ABT-122 and placebo. No deaths or serious AEs were reported for subjects dosed with ABT-122 or placebo IV, and no subject dosed with ABT-122 or placebo IV discontinued the study due to an AE. No AEs were reported during the 2-hour infusion of study drug and there were no reports of hypersensitivity reactions. The majority (18/21) of the events assessed were not related or probably not related to ABT-122. The three remaining events were assessed as possibly related to ABT-122 IV administration and were nausea, headache, and pruritus. All AEs were either mild (18 of the 21 AEs) or moderate (3 of the 21 AEs) in severity. Thus, data show similar AE profiles between placebo and ABT-122 (90.0% vs 70.0%) with IV dosing. The only AEs considered possibly related to ABT-122 IV were headache, nausea, and pruritus, These AEs were reported in 1 patient case each.

### Part 2—SC Dosing

Six of the 18 subjects (6/18, 33.3%) who received ABT-122 SC reported 1 or more AEs compared to the 1 subject (1/6, 16.7%) who received placebo SC. All AEs were reported by no more than 1 subject each in the ABT-122 group and placebo dose group. No deaths or serious AEs were reported for subjects treated with ABT-122 or placebo SC, and no subject administered ABT-122 or placebo SC discontinued the study due to an AE. There was one AE classified as a localized hypersensitivity reaction remote from the injection site in an ABT-122 treated subject, and there were no injection site reactions related to SC dosing. The majority of the AEs in the ABT-122 SC (5/6) assessed by the investigators were not related or probably not related to study drug. The AE of hypersensitivity was possibly related to ABT-122. All AEs were mild in severity. Similar AE profiles between placebo and ABT-122 (16.7% vs 33.3%) with SC dosing. The only AE even possibly related to ABT-122 was localized hypersensitivity reported in 1 subject (mild itching and erythema on cheeks and forehead).

No apparent dose relationship was observed in the frequency, type, or intensity of AEs for either the IV or SC dose groups in Study M12-704. Most AEs were assessed as either not related or probably not related to study drug by the investigator. No severe AEs or SAEs were observed. No subject died in the study or was discontinued from the study due to an AE.

### Adverse Events (AEs) of Special Interest

AEs of special interest were evaluated based on the toxicology profile of ABT-122 in nonclinical models, and the clinical profiles associated with anti-TNF and anti-IL-17 monotherapy, which included hypersensitivity reactions and infections.

### Hypersensitivity Reactions

Analysis of the AEs of special interest suggested that there were no AEs or patterns of AEs indicative of systemic hypersensitivity reaction. Howeverone female subject (0.3 mg/kg ABT-122 SC dose group) experienced an AE of localized hypersensitivity, described as an allergic skin reaction remote from the site of administration, which occurred approximately two hours after dosing on Study Day 1 and lasting a single day. The reaction was associated with mild itching, localized to both cheeks and the forehead, and described as mild erythema. The subject did not report any other systemic symptoms including gastrointestinal, respiratory, cardiovascular, or musculoskeletal symptoms, and her vital signs remained normal. This subject underwent protocol-directed activities for a possible hypersensitivity reaction, which included the collection of a panel of cytokines and complement factors at the time of her allergic skin reaction. This evaluation revealed no clinically relevant change in C-reactive protein, complement C3, complement C4, tryptase, CH50, IL-2, TNF, or IL-6 levels. No clinically relevant changes were noted in the laboratory values, including red blood cells, platelets, eosinophils and basophils for this subject. The subject recovered after two days without medical treatment. This event did not result in subject discontinuation from the study. This AE was not considered a systemic hypersensitivity reaction because there was only evidence for a single organ system (i.e., skin) involvement.

### Infections

The most common infectious AE reported was upper respiratory tract infection, which had a higher frequency within the placebo IV dose group compared to the ABT-122 IV dose group; observed in 5 (16.7%) subjects in the ABT-122 dose groups and 3 (30.0%) subjects in the placebo dose group. With the exception of the subcutaneous abscess, which was considered moderate in severity, the remaining events of infection were considered mild in severity. No AE was reported by 1 subject with placebo or ABT-122 SC dosing. Overall, there was no apparent dose response relationship in the rate of infectious AEs with ABT-122 therapy compared to placebo. The types and severity of infectious AEs observed are consistent with those that one would expect in the general healthy population.

### AE Profile Versus ADA Titer

In this study population, ADA was detectable in all subjects. No AEs were associated with the single-dose administration of ABT-122 DVD-Ig™ binding protein, regardless of ADA status.

### Laboratory, Vital Sign, and Electrocardiogram Evaluations

Overall there were no patterns of laboratory value changes or correlation with clinical AEs. The following laboratory related AEs were reported: two subjects reported AEs of blood creatine phosphokinase increased (0.1 mg/kg and 0.3 mg/kg IV ABT-122 dose groups), 1 subject each reported aspartate aminotransferase increased (0.3 mg/kg IV ABT-122 dose group), blood glucose increased (3.0 mg/kg IV ABT-122 dose group), and blood potassium increased (0.1 mg/kg IV ABT-122 dose group).

Although not judged to be clinically significant, the data in the ABT-122 IV and SC groups gave documentary evidence of an increasing effect on mean lymphocyte count and a decreasing effect on the neutrophil and monocyte counts, and perhaps on eosinophil count. There were no noted platelet effects or other formed blood elements effects observed during the study. Overall, data show no clinically significant changes in laboratory, vital sign, or electrocardiogram parameters. No infusion, systemic hypersensitivity, or injection-site reactions were observed.

### Adverse Drug Reaction Listing

Adverse drug reactions are defined as AEs experienced by subjects receiving ABT-122 that one considers expected for the purpose of determining expedited reporting requirements. At this time, no adverse drug reactions have been identified. All adverse drug reactions are considered as unexpected. ADAs observed in the study did not affect the safety/tolerability profile of ABT-122.

All treatment-emergent adverse events experienced by at least 1 subject receiving ABT-122, regardless of causality, include blood and lymphatic system disorders (neutropenia), cardiac disorders (palpitations), eye disorders (eye pruritus), gastrointestinal disorders constipation, diarrhoea (dyspepsia, glossodynia, nausea, vomiting), general disorders and administration site conditions (fatigue, injection site bruising, injection site rash, injection site reaction), hepatobiliary disorders (cholecystitis), immune system disorders (hypersensitivity, seasonal allergy), infections and infestations (body tinea, hordeolum), localised infection (nasopharyngitis, oral herpes, subcutaneous abscess, upper respiratory tract infection, urinary tract infection, viral infection, viral upper respiratory tract infection, vulvovaginal mycotic infection), injury, poisoning and procedural complications (arthropod bite, fall, muscle strain, post-traumatic pain, thermal burn, traumatic haematoma), investigations (aspartate aminotransferase increased, blood creatine phosphokinase increased, blood glucose increased, blood potassium increased, blood uric acid increased, hepatic enzyme increased), musculoskeletal and connective tissue disorders (arthralgia, back pain, muscle spasms, musculoskeletal discomfort, musculoskeletal pain, myalgia, pain in extremity), neoplasms benign, malignant and unspecified (cysts and polyps), rectal cancer, nervous system disorders (headache, paraesthesia, somnolence), psychiatric disorders (mood altered), renal and urinary disorders (dysuria), respiratory, thoracic and mediastinal disorders (cough, nasal congestion, oropharyngeal pain), skin and subcutaneous tissue disorders (dermal cyst, dry skin, erythema, pruritus, rash), and vascular disorders (hypertension, phlebitis). No events have resulted in death.

Summary of Data from Study M12-704: Single Ascending Dose Study in Normal Healthy Volunteers

A total of 48 healthy subjects received active treatment with ABT-122 IV (0.1 to 10 mg/kg IV) or SC (0.3 to 3 mg/kg SC) in this randomized, DB, single ascending dose study (Study M12-704). There were no SAEs or discontinuations due to AEs in the study and no dose limiting toxicities were identified. No patterns of AEs appeared to be dose-related or associated with route of administration (IV or SC). The incidence of AEs was lower following SC administration compared to the IV route. No systemic hypersensitivity reactions, infusion reactions or injection site reactions occurred. There were no observed effects on platelets, or bone marrow derived formed elements. The rate of reported infections among the treatment groups was not dose related and the types of infections observed are common in the healthy general population. Abnormal laboratory values observed were mostly asymptomatic, transient and did not require medical intervention. The analysis of ABT-122 safety data supported the further investigation of ABT-122 in subjects with RA.

### Example 5

### Study M14-346—Bioavailability, Safety and Tolerability of 100 mg/Ml of ABT-122

Study M14-346, a Phase 1, randomized, parallel group study in healthy adult subjects evaluated the bioavailability, safety and tolerability of a higher concentration formulation (100 mg/mL) of ABT-122 compared to the current lower concentration formulation (50 mg/mL) of ABT-122. Study drug was administered in the morning on Study Day 1 as follows: Regimen A (N=12) was a single dose of 1.5 mg/kg using 50 mg/mL low concentration formulation (Reference). Regimen B (N=12) was a single dose of 1.5 mg/kg using 100 mg/mL high concentration formulation (Test).

### Adverse Effects

Adult subjects (23 males and 1 female) were enrolled, received study drug regimen according to their randomized assignment, and completed the study. A total of 9/24 (37.5%) subjects reported at least 1 treatment-emergent AE. A total of 12 treatment-emergent AEs by MedDRA term were reported during the 57-day post dosing monitoring period. All AEs were mild in severity with the exception of a single Grade 3 AE of neutropenia occurring in 1 subject who received Regimen B (high concentration). This subject had a Grade 1 neutropenia (1520 cells/mm³) at baseline prior to dosing. The neutrophil levels decreased to 770 cells/mm³ on Study Day 2, approximately 24 hours after dosing, corresponding to Grade 3 neutropenia, and returned to baseline value on Study Day 8. Throughout the remainder of the study (Study Days 11 through 56), the absolute neutrophil count ranged from 860 to 1970 cells/mm³. The subject demonstrated no clinical signs or symptoms of illness, other significant laboratory findings, vital signs abnormalities, ECG changes, or physical examination findings. The subject remained afebrile and was otherwise well throughout the course of the study and had no other reported AEs during the 57-day post dosing period.

Five of the 12 AEs reported during the study were considered to have a reasonable possibility of being related to study drug. All five were in subjects receiving treatment Regimen B (high concentration) and consisted of Grade 1 injection site reactions in three subjects, Grade 1 headache in 1 subject, and the Grade 3 neutropenia described above. All three of the injection site reactions were localized and resolved without treatment within 2.5 hours.

There were no hypersensitivity reactions or severe infections reported in the study. All infections reported were mild in nature and resolved by standard therapy. No deaths, SAEs, or discontinuations due to AEs occurred during the study.

### Laboratory, Vital Sign, and Electrocardiogram Evaluations

Six subjects (one subject in Regimen A and five subjects in Regimen B) had hemoglobin values that met potentially clinically significant criteria ≧Grade 2. Upon review of the data, the values were not considered to be clinically significant and were not as sociated with any AEs. The Grade 3 neutropenia occurring on Study Day 2 was the only hematology variable reported as an AE. Values outside the reference ranges in serum chemistry and urinalysis variables reported after study drug administration were of an isolated nature or were already present at baseline, and were not considered by the investigator to be associated with study drug administration or clinically significant.

No potentially clinically significant values were observed during the study for any of the vital signs parameters. No subject had a QTc interval of more than 500 milliseconds (msec) or an increase in QTc of more than 60 msec from baseline. No AEs were associated with any of the ECG changes throughout the study.

### Pharmacokinetics

Study M14-346 was a Phase 1, single-dose, randomized, parallel-group, open-label, single-center study in healthy volunteers to evaluate the bioavailability of a high concentration (100 mg/mL to be used in Phase 2a study) relative to that of the low concentration formulation (50 mg/mL) utilized in Phase 1. Twenty-four subjects who fulfilled the eligibility criteria were equally randomized to receive a single 1.5 mg/kg SC dose of either the high- or low-concentration formulations (N=12 per arm). Serial PK samples were collected for 57 days. The bioavailability after a single 1.5 mg/kg SC dose of the 100 mg/mL test formulation of ABT-122 relative to that of the 50 mg/mL ABT-122 reference formulation was 80% for Cₘₐₓ and 83% for AUC.

### Example 6

### Study M12-962—Phase 1 Analysis of Human Patients with RA and Methotrexate

Study M12-962 is a Phase 1, randomized, DB, placebo-controlled, multiple dose study designed to assess the safety, tolerability, pharmacokinetics, and immunogenicity of varying doses of ABT-122 given with MTX to subjects with RA in three dose groups which have completed dosing, and without MTX to subjects with psoriasis in one dose group.

In Group 1, ABT-122 (or placebo) was administered once every other week (EOW) for a total of four doses to subjects with RA. In Groups 2 and 3, ABT-122 (or placebo) was administered once a week for a total of eight doses to subjects with RA. Subjects with RA continued on their stable dose of MTX weekly throughout participation in the study. In Group 4, ABT-122 (or placebo) is being administered once every week (EW) for a total of eight doses to subjects with psoriasis, without concomitant MTX treatment. The tested ABT-122 doses in study M12-962 are shown in Table 14.

TABLE 14
Dose Groups In Study M12-962

| | | Planned Number |
|---|---|---|
| | | Of Subjects |
| Group^{a} | Regimen^{b,c} | Active:Placebo^{a} |
| Group 1 | 1.0 mg/kg ABT-122 or placebo SC, | 6:2 |
| | once every other week | |
| Group 2 | 1.5 mg/kg ABT-122 or placebo SC, | 6:2 |
| | once a week | |
| Group 3 | 3.0 mg/kg ABT-122 or placebo SC, | 6:2 |
| | once a week | |
| Group 4^{a} | 1.5 mg/kg ABT-122 or placebo SC, | 6:2 |
| | once a week | |

^{a}Subjects do not participate in more than 1 dosing group.
^{b}Dose level or dosing frequency adjustment allowed based on the available safety, tolerability, and PD data from previous dosegroups.
^{c}Subjects with RA also received their stable MTX dose weekly.
d. In practice in the first 3 groups, 7 subjects (instead of 6) were randomized and received at least 1 placebo dose. One subject in the placebo group withdrew consent and, in accordance with the protocol, was replaced following agreement between the investigator and AbbVie.
e. Subjects with psoriasis in Group 4 do not receive MTX during the study.

Preliminary safety data are provided for Groups 1, 2, and 3, which was conducted in subjects with RA receiving their stable background MTX dose weekly. Overall there were no patterns of AEs or dose relationship of AEs with ABT-122. At least one treatment-emergent AE was reported by 5/7 (71.4%) placebo recipients and by four subjects (66.7%) in the 1.0 mg/kg and 1.5 mg/kg ABT-122 dose groups, and by three subjects (50%) in the 3.0 mg/kg ABT-122 dose group. Headache, reported by four ABT-122 recipients (two each in the 1.5 mg/kg and 3.0 mg/kg dose groups) and by two placebo recipients, was the most commonly reported AE during the study. None of these cases was considered to be ABT-122-related. Oral herpes reported by one subject in the 1.5 mg/kg dose group was the only AE that had a reasonable possibility of being related to ABT-122 and MTX. Of the 11 subjects in the ABT-122 dose groups with AEs, seven experienced Grade 2 (moderate) AEs, three experienced Grade 1 (mild) AEs, and one experienced a Grade 3 (severe) AE. The Grade 3 (severe) AE occurred in the ABT-122 1.5 mg/kg group (rectal cancer with diagnosis on Study Day 65, after completion of 8 weekly doses). The Grade 3 rectal cancer led to discontinuation of the subject from further study participation as he had already completed dosing. The AE of rectal cancer was assessed by the investigator as not related to study drug. Of the five placebo recipients reporting AEs, three experienced Grade 2 (moderate) AEs and two experienced Grade 1 (mild) AEs. No death or SAE occurred in this study.

### Laboratory, Vital Sign, and Electrocardiogram Evaluations

Overall there were no clinically relevant patterns of laboratory abnormalities or associated clinically relevant events. Hematology parameters meeting the criteria for potentially clinically significant values were observed in nine subjects. All were isolated, single occurrences with the exception of Grade 2 decreased hemoglobin recorded on Study Day 15 and Study Days 36 to 64 (one subject in the ABT-122 1.5 mg/kg dose group), returning to normal range at the last study test on Day 74.

Chemistry variables meeting the criteria for potentially clinically significant values were observed in eight subjects (three in the Placebo group, 2 each in the ABT-122 1.0 mg/kg and 3.0 mg/kg dose groups, 1 in the ABT-122 1.5 mg/kg dose group). All were Grade 2 with the exception of Grade 3 elevated ALT seen on Day 43 in a subject in the ABT-122 3.0 mg/kg group. All serum chemistry values ≧Grade 2 were isolated events occurring on 1 day only, or were present at baseline, or, if observed on multiple days, were not associated with other potentially clinically significant laboratory results or any AE. No potentially clinically significant urinalysis variables were observed.

No potentially clinically significant values were recorded during the study for any of the vital signs parameters. ECG results identified as clinically significant abnormal were reported for one subject (data remain blinded for treatment). A PR interval >220 msec was recorded on a single 12-lead ECG with no association with clinical symptoms or other clinically relevant ECG parameter. No QT interval was identified as prolonged by the local reader; no value ≧500 msec was recorded.

### Example 7

### Study M14-048—Phase 1 Analysis of Human Patients with RA and Methotrexate

Study M14-048 was a Phase 1, randomized, DB, placebo-controlled, multiple dose study designed to assess the safety, tolerability, PK, and immunogenicity of varying doses of ABT-122 given with MTX (Table 15). The study enrolled subjects with a diagnosis of RA who had been on stable dose of MTX for greater than or equal to (>) four weeks. The protocol also included an exploratory assessment of the disease response for multiple SC injections of ABT-122 in subjects with RA. The study is complete and the analyses underway.

Nineteen adult subjects (twelve females and seven males) were enrolled and received study drug regimen according to their randomized assignment with six subjects receiving placebo and 13 subjects receiving ABT-122. There were no patterns of AEs evident, nor evidence of a dose relationship with the safety profile. There were only two AEs which were reported in more than one subject, injection site bruising (two subjects in the 1.5 mg/kg group) and headache (two subjects in the 1.5 mg/kg group).

TABLE 15
Dose Groups In Study M14-048

| | | Number of Subjects |
|---|---|---|
| Group^{a} | Regimen^{b} | Active:Placebo |
| 1^{c} | 0.5 mg/kg ABT-122 or placebo SC, | 6:2 |
| | once a week | |
| 2 | 1.5 mg/kg ABT-122 or placebo SC, | 6:2 |
| | once a week | |
| 3 | 3.0 mg/kg ABT-122 or placebo SC, | 6:2 |
| | once a week | |

^{a}Subjects may not participate in more than one dosing group.
^{b}Subjects receive their stable MTX dose weekly.
^{c}Group 1 enrolled 2 subjects only.

The intensity of all treatment-emergent AEs was categorized as Grade 1 (mild) with the exception of Grade 2 (moderate) AEs reported by four subjects in the Placebo group (toothache, injection site reaction, local swelling, and ankle fracture), one subject in the ABT-122 1.5 mg/kg group (hypertension), and 3 subjects in the ABT-122 3.0 mg/kg group (injection site reaction, cholecystitis, upper respiratory tract infection).

The Grade 2 cholecystitis event occurring in one subject in the ABT-122 3.0 mg/kg group resulted in an unscheduled cholecystectomy and was the only SAE reported during the study. This event was considered not related to study drug by the investigator. Grade 2 injection site reactions were reported in two subjects after the second dose of study drug (one placebo recipient and one subject in the ABT-122 3 mg/kg dose group), both self-limited without sequelae resulted in treatment discontinuation of both subjects.

Other treatment-emergent AEs with a reasonable possibility of being related to study drug as assessed by the investigator were one case each of nausea, injection site reaction, decreased appetite, and lethargy in the Placebo group; two cases of injection site bruising and one case each of rash and hypertension in the ABT-122 1.5 mg/kg group. No treatment-emergent AE was attributed a reasonable possibility of being related to MTX by the investigator.

There were no systemic hypersensitivity reactions or severe infections that occurred in the study. All infections reported were mild in nature and resolved by standard therapy. No deaths occurred during the study.

### Laboratory, Vital Sign, and Electrocardiogram Evaluations

Overall there were no patterns of laboratory value changes or evident dose relationship. No changes in hematology values were reported as AEs. Seven subjects (one placebo recipient and six ABT-122 recipients, one subject in the 1.0 mg/kg dose group, three subjects in the 1.5 mg/kg dose group, and two subjects in the 3.0 mg/kg dose group) had decreased hemoglobin values that met potentially clinically significant criteria ≧Grade 2. Lymphocyte counts met potentially clinically significant criteria ≧Grade 2 in four subjects in the Placebo group and 3.0 mg/kg groups, five subjects in the ABT-122 1.5 mg/kg and in the single subject in the ABT-122 1.0 mg/kg group. However, in eleven of the total 14 subjects, the value was seen prior to dosing (all four Placebo recipients, three subjects in 1.5 mg/kg group and all four subjects in the 3.0 mg/kg dose group).

Grade 2 neutrophil counts were seen in four subjects (2 each in the ABT-122 1.5 mg/kg and 3.0 mg/kg dose groups). All four cases were single isolated events.

A single subject in the 1.5 mg/kg dose group, experienced Grade 2 and 3 elevated alanine aminotransferase (ALT) onset Day 36 and Day 47, respectively, Grade 2 elevated aspartate aminotransferase (AST) onset Day 43, which were still at a Grade 2 on Day 91 and Grade 2 elevated creatine phosphokinase (CPK) were seen on Days 24 and 25. The laboratory events of Grade 1 (mild) elevated hepatic enzyme and blood uric acid were reported as treatment-emergent AEs in this subject. Neither event was considered to have a reasonable possibility of being study drug related by the investigator nor was associated with any reported clinical symptoms. There were no other clinically relevant serum chemistry or urinalysis values reported. No vital signs parameters recorded for any subjects during the study met the criteria for potentially clinically significant. However, Grade 2 (moderate) hypertension was reported as an ABT-122-related AE in one subject (1.5 mg/kg group). No other AE was reported by this subject. No ABT-122 recipient had a QTc interval of more than 470 msec or an increase in QTc of more than 30 msec.

There have been no reported SAEs, deaths, or severe AEs from preliminary blinded data from Groups 1 and 2. Dosing has advanced to subjects in Group 3 at 3 mg/kg every week.

Summaries of the preliminary multiple dose PK results from Studies M12-962 and M14-048 are shown in Table 16 (following multiple doses).

TABLE 16
ABT-122 Pharmacokinetic Parameters (Mean, Coefficient of Variation) Following
Multiple Escalating SC ABT-122 Doses in Studies M12-962 and M14-048

| | 0.5 mg/kg EW | 1 mg/kg EOW | | 1.5 mg/kg EW | | 1.5 mg/kg EW | | 3 mg/kg EW | | 3 mg/kg EW | |
|---|---|---|---|---|---|---|---|---|---|---|---|
| | (Study M14-048) | (Study M12-962) | | (Study M14-048) | | (Study M12-962) | | (Study M12-962) | | (Study M14-048) | |
| | 8^{th} Dose | 4^{th} Dose | | 8^{th} Dose | | 8^{th} Dose | | 8^{th} Dose | | 8^{th} Dose | |
| Parameter (Units) | (N = 1)^{a} | (N = 6) | | (N = 5)^{b} | | (N = 5)^{c} | | (N = 6) | | (N = 5)^{d} | |
| Cₘₐₓ (μg/mL) | 2.69 | 5.96 | (37) | 27.3 | (16) | 25.7 | (50) | 54.7 | (20) | 80.9 | (27) |
| Tₘₐₓ (day)^{e} | 1.5 | 3 | (1-4) | 4 | (1.5-7) | 2 | (0-4) | 2 | (2-4) | 2 | (1.5-7) |
| AUCₜₐᵤ (μg · day/mL) | 17.8 | 60.5 | (39) | 172.6 | (17) | 137.9 | (43) | 308.5 | (20) | 448.2 | (21) |
| C_{trough} (μg/mL) | 2.49 | 2.31 | (64) | 23.8 | (25) | 19.0 | (68) | 41.2 | (30) | 60.0 | (36) |
| t½ (day)^{f} | 5.11 | 5.8 | (56) | 11.5 | (40)^{g} | 11.4 | (22)^{h} | 11.1 | (23) | 8.9 | (20) |
| CLₛₛ/F (L/day) | 2.24 | 1.6 | (59) | 0.71 | (17) | 1.01 | (45) | 0.81 | (21) | 0.55 | (20) |
| Cₘₐₓ/Dose (μg/mL/mg/kg) | 5.38 | 5.96 | (37) | 18.2 | (16) | 17.1 | (50) | 18.2 | (20) | 27.0 | (27) |
| AUCₜₐᵤ/Dose | 35.7 | 60.5 | (39) | 115.0 | (17) | 92.0 | (43) | 102.8 | (20) | 149.4 | (21) |
| (μg · day · mL/mg/kg) | | | | | | | | | | | |
| C_{trough}/Dose (μg/mL/mg/kg) | 4.98 | 2.31 | (64) | 15.8 | (25) | 12.7 | (68) | 13.7 | (30) | 20.0 | (36) |

| | | | | | | |
|---|---|---|---|---|---|---|
| AUCₜₐᵤ Racⁱ | 2.55 | 1.57 | 4.15 | 3.79 | 5.64 | 5.39 |
| | | (0.79-2.95) | (3.03-7.19) | (3.11-10.2) | (2.93-10.05) | (2.87-7.16) |

EW = every week;
EOW = every week
^{a}Group 1 (0.5 mg/kg EW) of Study M14-048 enrolled only 2 subjects (1 active, 1 placebo) since adequate data on an equivalent dose level (1 mg/kg every 2 weeks) was already obtained from Study M12-962.
^{b}One subject missed the 6^{th} ABT-122 dose due to an infection event and was excluded from the summary of PK parameters.
^{c}One subject did not receive the 7^{th} ABT-122 dose due to moderate common cold and was excluded from the summary of the pharmacokinetic parameters.
^{d}One subject was discontinued from the study after the 2^{nd} ABT-122 dose due to an injection site reaction, and 1 subject had a drug holiday after the 3^{rd} ABT-122 dose due to unplanned surgery. Both subjects were excluded from the summary of the PK parameters after the 4^{th} dose, and only Subject 3001 was excluded from the summary PK parameters after the 8^{th} ABT-122 dose.
^{e}Median (range).
^{f}Harmonic mean and PseudoCV. g.
N = 6.
^{h}N = 4; in addition to the above exclusion, one subject was excluded from the summary statistics of half-life due to lack of washout data after the 8^{th} ABT-122 dose.
ⁱR_{ac} = Accumulation ratio (Cₘₐₓ, 4^{th} or 8^{th} Dose/Cₘₐₓ, 1^{st} Dose or AUCₜₐᵤ, 4^{th} or 8^{th} Dose/AUCₜₐᵤ, 1^{st} Dose).
Note:
Tau is 14 days for EOW and 7 days for EW regimens.

Steady-state appears to be established within 4 to 5 weeks of dosing, and ABT-122 steady-state exposure appears to be proportional to dose at the evaluated dose range. ABT-122 Cₘₐₓ to C_{trough} ratio is 2.6 after bi-weekly dosing and 1.3 after the weekly dosing regimen; the corresponding mean effective t1/2 is 10 to 34 days when co-administered with MTX. Overall, detectable ADA titers were observed in 58% of subjects (18/31) who received ABT-122 in the multiple ascending dose studies. In the majority of subjects with detectable ADA, the ADA titer values were close to the lower limit of detection (10 titer units) and did not appear to influence ABT-122 exposure. One subject developed high ADA titer which was associated with loss of ABT-122 exposure.

### Safety

The potential safety concerns for administration of ABT-122 are the risk of systemic hypersensitivity reactions and an increased risk for infection. Although there was no evidence for either of these safety concerns in Study M12-704, several precautions were taken in Study M12-962 and Study M14-048 to mitigate the risk of potential systemic hypersensitivity reactions with ABT-122. To address the risk for infection or hypersensitivity reactions in humans who receive ABT-122, study protocols implement enrollment criteria, screening procedures, and the clinical schedule and monitoring plan to mitigate, monitor, and manage potential hypersensitivity reactions, other systemic reactions, and infections.

The risk of other AEs that have been associated with the anti-TNF agents, including malignancy, central nervous system demyelinating disease, pancytopenia (including aplastic anemia), worsening or new onset heart failure, and lupus-like syndrome, is low given the limited duration of exposure in this study, and application of protocol-specified exclusion criteria, and safety monitoring procedures in both Study M12-962 and Study M14-048.

No systemic hypersensitivity reactions have been seen in Study M12-962 or Study M14-048 in subjects with RA receiving multiple ABT-122 doses with weekly background MTX. In FIH Study M12-704, healthy volunteers were given single doses of ABT-122 DVD-Ig™ binding protein without concomitant MTX. There were no systemic hypersensitivity reactions seen in subjects given a single dose of ABT-122 including some subjects with positive pre-dose ADA titers in Study M12-704.

Occasional infections were reported during the course of the studies. Overall, there was no apparent dose response relationship in the rate of infectious AEs with ABT-122 therapy compared to placebo. The types and severity of infectious AEs observed have been consistent with those that one would expect in the general population similar to study subjects. Although not judged to be clinically significant, the data in the single-dose groups gave evidence of an increasing effect on mean lymphocyte count and a decreasing effect on the neutrophil and monocyte counts, and perhaps on eosinophil count. There were no observed platelets or other formed blood elements effects observed during either study.

Subsequent multiple dose administration in subjects with RA have not found dose limiting toxicities and apparent association of particular AEs with dose. There has been a high prevalence of detectable ADA across the dose groups, with the majority of subjects exhibiting a low titer. For most subjects, the presence of ADA did not influence drug clearance and did not correlate with any systemic or AE profiles.

The risk for infections clinical study thus far has been minimized due to the short dosing duration, close monitoring of subjects, and enrollment criteria excluding subjects who might have a greater propensity for infection.

Available Pharmacokinetic and Dosing Data

ABT-122 is a high-affinity recombinant human molecule with TNF-binding properties comparable to those of the monoclonal anti-TNF antibody adalimumab. Affinities for TNF are 8 pM with ABT-122, and 30 pM with adalimumab (Kamyakcalan et al. (2009) Clin. Immunol. 131(2):308-316). In a Phase 1 clinical trial of patients with RA treated with a single IV dose of adalimumab, a clinical response was observed at the lowest dose tested, 0.5 mg/kg, with greater response observed at all higher doses tested, up to 10 mg/kg. In a Phase 3 study of multiple, SC doses of adalimumab monotherapy for RA, a successively greater clinical response was observed with increasing exposure in doses corresponding to a dose range of approximately 0.3 to 1.14 mg/kg (van de Putte et al. (2004) Ann. Rheum. Dis. 63(5):508-516). This range encompasses the indicated starting dose for adalimumab in RA, 40 mg EOW, which corresponds to approximately 0.6 mg/kg.

Neutralization of IL-17 has been clinically examined with 2 monoclonal antibodies, AIN457 (secukinumab) and LY2439821 (ixekizumab). See Table 17 and Genovese et al. (2010) Arthr. Rheum. 62(4):929-939). In Phase 1 studies in patients, AIN457 showed evidence of clinical effect at 3 mg/kg (for treatment of psoriasis) and 10 mg/kg (for the treatment of RA and uveitis), while LY2439821 showed evidence of clinical effect in RA with multiple weekly doses ranging from 0.2 to 2.0 mg/kg (Hueber et al. (2010) Sci. Transl. Med. 2(52):52ra72; Genovese et al. (2010) Arthr. Rheum. 62(4):929-939). Subsequent published results of long-term 52-week Phase 2 studies in both secukinumab and ixekizumab have demonstrated an acceptable safety profile for IL-17 inhibition, without evidence of an increased signal of SAEs.

Data compiled from studies M12-962 and M14-04 that show that ABT-122 demonstrated effective dose-dependent responses in rheumatoid patients with stable disease. The compiled data show changes in baseline values for hsCRP, neutrophils and DAS28-CRP. Data shows that both hsCRP (a systemic marker of inflammation) and DAS28-CRP (a clinical Disease Activity Score) are TNF and IL-17 responsive.

CXCR4 is a receptor for CXC chemokine family involved in immune cell trafficking. CXCR4 gene expression is upregulated in rheumatoid arthritis. Combination of TNF+IL-17 increased CXCR4 in rheumatoid joint synovial fibroblasts. However, a TNF inhibitor alone does not modulate CXCR4 on T cells in RA. A decreased in vivo CXCR4 expression was observed on multiple immune cells from samples collected from ABT-122 treated patients compared to control patients not administered ABT-122. A 52%, 16% and 45% decrease in CXCR4 expression was observed for B cells, monocytes and T cells. These early studies have demonstrated an acceptable safety profile for IL-17 inhibition, without evidence of an increased signal of SAEs.

Overall, these results indicate that the dose range evaluated for repeat dose regimens of ABT-122, 0.5 mg/kg weekly to 3.0 mg/kg weekly, encompasses the range that has been assessed for monospecific antibodies targeting IL-17, although it is not known whether the anti-IL-17 activity of ABT-122 in humans will resemble that of either of these comparator molecules.

Together, the available data for TNF and IL-17 inhibitors, along with the safety and PK profile of ABT-122 through the regimen of about 1.5 mg/kg administered once weekly support further clinical studies of ABT-122 DVD-Ig binding protein.

TABLE 17
Phase 1 Dose Levels For Comparator
Monoclonal Antibodies (mAbs)

| Target | Compound | Sponsor | IV Dose Levels, Applications |
|---|---|---|---|
| TNF | adalimumab | Abb Vie | 0.5, 1, 3, 5, 10 mg/kg; |
| | | | RA |
| IL-17 | AIN457 | Novartis | 3 mg/kg, psoriasis; 10 mg/kg; |
| | (secukinumab) | | RA and uveitis |
| IL-17 | LY2439821 | Eli Lilly | 0.06, 0.2, 0.6, 2.0 mg/kg; |
| | (ixekizumab) | | RA |

Current pharmacokinetic data from Phase 1 study of multiple SC doses of ABT-122 in subjects with RA support the dosing regimen up to every other week and continued Phase 2 development. Following multiple ABT-122 doses ranging from 0.5 to 3.0 mg/kg in subjects with RA, steady-state appears to be established within 4 to 5 weeks of dosing, and ABT-122 steady-state exposure appears to be proportional to dose at the evaluated dose range. ABT-122 Cmax to Ctrough ratio is 2.6 after bi-weekly dosing and 1.3 after weekly dosing (the corresponding mean effective half-lives of 10.2 and 16.1 to 34.6 days, respectively, when co-administered with MTX).

Preliminary data from the evaluation of the bioavailability of a high concentration (100 mg/mL to be used in Phase 2a study) relative to that of the low concentration formulation (50 mg/mL) utilized in Phase 1 has shown the bioavailability after a single 1.5 mg/kg SC dose of the 100 mg/mL test formulation of ABT-122 relative to that of the 50 mg/mL ABT-122 reference formulation was 80% for Cmax and 83% for AUC. In addition, there was no clinically significant adverse effect on the safety and tolerability of ABT-122 following a single dose of the higher concentration formulation.

Overall, detectable ADA titers are observed in 58% of subjects (18/31) who received ABT-122 in the multiple ascending dose studies. ADA titer values were principally close to the lower limit of detection (10 titer units) and did not appear to influence ABT-122 exposure.

### Example 8

### Study M12-963—Phase 2 Study to Investigate the Safety and Efficacy of ABT-122 Administered with Methotrexate in Subjects with Active Rheumatoid Arthritis Who have an Inadequate Response to Methotrexate

A study M12-963 is performed primarily to estimate the effect of ABT-122 by measuring the percentage of subjects achieving an American College of Rheumatology 20% (ACR20) response at Week 12, and to assess the safety and tolerability of ABT-122 in subjects with RA. The study involves analysis efficacy of ABT-122 administered with methotrexate in subjects with active RA who have an inadequate response to methotrexate. Study M12-963 explores a range of ABT-122 exposures (120 EW, 120 EOW or 60 EOW) to determine whether the regimen is comparable to different adalimumab (Humira®) exposures, e.g., 40 mg EW or 40 mg EOW.

Secondary objectives include the determination of the ACR20, ACR50, and ACR70 responder rates at Weeks 2, 4, 8 and 12; change from Baseline in Physician Global Assessment of Disease Activity (VAS) at Weeks 2, 4, 9 and 12; change from Baseline in Patient Reported Outcomes at Weeks 2, 4, 8, and 12 (e.g., using a Health Assessment Questionnaire (HAQ-DI); and a patient global assessment of disease activity (VAS)); pharmacokinetics of multiple dosing of ABT-122 in subjects with RA; and measurement of ADA following multiple SC injections of ABT-122 in subjects with RA.

Exploratory objectives include an analysis of the following: change from Baseline in tender joint count (TJC) at Weeks 2, 4, 8 and 12; change from Baseline in swollen joint count (SJC) at Weeks 2, 4, 8 and 12; change from Baseline in patient's assessment of pain (VAS) at Weeks 2, 4, 8 and 12; change from Baseline in Work Instability Scale for RA (WIS) at Weeks 2, 4, 8 and 12; change from Baseline in Short Form Health Survey (SF-36) at Weeks 2, 4, 8 and 12; ACRn at Week 12; proportion of subjects achieving Low Disease Activity (LDA) using various parameters (i.e., 2.6≦Disease Activity Score 28 (DAS28) based on C-reactive protein (CRP)<3.2; or 2.8<Clinical Disease Activity Index (CDAI)<10; or 3.3<simple disease activity index (SDAI)<11) at Week 12 and Week 16 (for those subjects who do not participate in the Open

Label Extension); proportion of subjects achieving Clinical Remission using various parameters (i.e., DAS28 (CRP)<2.6, or CDAI≦2.8, or SDAI <3.3) at Week 12 (for subjects who do not participate in the OLE); change from Baseline in DAS28 (CRP) at Weeks 2, 4, 8 and 12; change from Baseline in CRP at Week 2, 4, 6, 8 and 12; and to explore the pharmacokinetic/pharmacodynamic relationship.

The study is a multicenter investigation involving approximately 75 study sites globally. The study population includes adult female and male human subjects who are at least 18 years of age, have a diagnosis of RA for at least 6 months and have not responded adequately to methotrexate (MTX) treatment. The number of subjects enrolled is approximately 120 human patients.

This study is a randomized, double-blind, double-dummy, parallel-group, active-controlled study designed to assess the safety, tolerability and efficacy of multiple doses of the ABT-122 in subjects with active RA who are inadequately responding to MTX treatment (FIG. 14). The study is conducted in approximately 120-160 subjects.

The study is a 16 to 24 week study. The study includes a 30-day screening period (including 2 visits: Screening [within 30 days prior to the first dose of study drug] and a Baseline Visit [within 2 days prior to the first dose of study drug]); and a 12 week double-blind, active-controlled treatment period. See also Table 18.

Study visits occur at Screening, Baseline, Day 1, and weekly through Week 12. Upon completion of the study (Week 12 study visit), subjects responding to treatment are offered the option to continue receiving study drug in an open label extension study, with a primary objective to evaluate the long-term safety, tolerability and efficacy of the ABT-122 in patients with RA. Subjects who decide not to participate in or do not meet the selection criteria for the open-label extension study have a follow-up visit occurring at approximately 4 weeks following receipt of his/her last dose of study drug (Week 16) and a phone call 4 weeks later (Week 20) (Follow-up Period).

On Day 1, the eligible subjects are randomized in a 1:1:1:1 fashion to one of three doses of ABT-122 or adalimumab (Humira®). The human subject completes baseline procedures within two days (Baseline Visit) prior to the administration of the first dose of study drug. The Baseline Visit procedures are conducted on Day 1 prior to administration of the first dose of study drug.

The following are the dosing arms: ABT-122 low-dose: administered every week (EW); the ABT-122 mid-dose: administered EW; ABT-122 high dose: administered EW; or adalimumab (Humira®) 40 mg administered every other week (EOW). Each of these are administered by subcutaneous injection.

In other studies, the following are the dosing arms for ABT-122: ABT-122 60 milligram (mg) dose: administered every week (EW); ABT-122 120 mg dose: administered EW; ABT-122 120 mg dose: administered EW; or adalimumab (Humira®) 40 mg dose administered every other week (EOW). Each of these are administered by subcutaneous injection. FIG. 14 shows a study schematic used in the current example including the 30 days screening period, then 12 week double blind treatment period, and a follow-up visit period after six weeks and 70 days after the last treatment visit. See also Table 18.

Subjects receive study drug on a weekly (EW) or every other week (EOW) basis. For the adalimumab arm, subjects receive matching placebo EOW, alternating with the study drug. Subjects are required to return to the investigator site for study drug administration and stay for a minimum of 2 hours post injection for observation. Randomized subjects receive study medication for up to 12 weeks (Treatment Period). Subjects who complete 12 weeks of the study have an opportunity to enter Part 2 of the study where they receive a higher dose of ABT-122 for 36 weeks.

### Use of Concomitant Medications:

Subjects who are enrolled in the study must have been on MTX therapy for >3 months and on a stable prescribed dose of MTX for at least 4 weeks prior to the first dose of study drug. Subjects continue taking MTX as prescribed in addition to receiving study drug (ABT-122 or adalimumab). Reduction in the dose of MTX is not allowed. If the subject cannot tolerate his or her dose of MTX, he/she is discontinued from the study.

Subjects are required to use folic acid (or the equivalent) during the study, with the dose and regimen chosen per investigator's judgment. If the subject is already taking folic acid or the equivalent, the dose should remain stable through study participation.

TABLE 18
Study information and Treatments administered for Study M12-963

| Investiga- | ABT-122 100 mg Powder | Placebo for ABT-122 Powder |
|---|---|---|
| tional Product | for solution for Injection Vial | for solution for Injection Vial |
| Dosage Form | Lyophilized powder for injection in | Lyophilized powder for injection |
| | vials | in vials |
| Formulation | ABT-122, Sucrose, Histidine, | Sucrose, Etistidine, |
| | Polysorbate 80, Water for | Polysorbate 80, Water for |
| | injections, Hydrochloric Acid | injections, Hydrochloric Acid |
| | added as necessary to adjust pH | added as necessary to adjust pH |
| Strength | 100 mg/mL when reconstituted | N/A when reconstituted with |
| (mg) | with 1.2 mL of sterile water for | 1.2 mL of sterile water for |
| | injection | injection |
| Mode of | Subcutaneous injection | Subcutaneous injection |
| Administration | | |
| Investiga- | Adalimumab 40 mg | Placebo for Adalimumab 40 mg |
| tional Product | Pre-filled Syringe | Pre-filled Syringe |
| Dosage Form | Solution for injection in pre-filled | Solution for injection in pre-filled |
| | syringe | syringe |
| Formulation | Adalimumab/Mannitol, Citric acid | Mannitol, Citric acid monohydrate, |
| | monohydrate, Sodium citrate, | Sodium citrate, Disodium |
| | Disodium phosphate dihydrate, | phosphate dihydrate, Sodium |
| | Sodium dihydrogen phosphate | dihydrogen phosphate dihydrate, |
| | dihydrate, Sodium chloride, | Sodium chloride, Polysorbate 80, |
| | Polysorbate 80, Water for | Water for injections, Sodium |
| | injections, Sodium Hydroxide | Hydroxide added as necessary to |
| | added as necessary to adjust pH | adjust pH |
| Strength | 40 mg/0.8 mL | N/A |
| (mg) | | |
| Mode of | Subcutaneous injection | Subcutaneous injection |
| Administration | | |

ABT-122 100 mg powder for solution for injection vial and matching placebo for ABT-122 100 mg powder for solution for injection vial are reconstituted with sterile water for injection.
Mode of administration for adalimumab and placebo for adalimumab is subcutaneous injection. Adalimumab 40 mg/0.8 mL and placebo for adalimumab 0.8 mL do not require any reconstitution before use.

### Collection of Blood Samples for Pharmacokinetic Assessments:

Blood samples from all subjects enrolled are taken to measure pharmacokinetic variables.

All subjects have blood drawn for pharmacokinetic assessment at specified study visits collected pre-dose (0 hour), no more than 30 minutes before the dose as trough plasma concentrations (Ctrough). In addition, a subset of subjects (approximately 30%) participate in a more extensive pharmacokinetic investigation, where serial PK samples are drawn pre-dose (0 hour) on Day 1 and at the following time points post-dose on Day 1 and Week 12 visits: 2 hours and 72 to 120 hours.

Blood samples are collected to assess the mechanism of action of ABT-122 and a disease response. Samples are analyzed for measurement of non-genetic markers related to disease activity/prognosis of RA, autoimmunity/inflammation, and/or response to anti-RA medications, including ABT-122 or drug of this class. Blood samples are also collected to determine the presence of ADA and measurement of ADA titers for the assessment of immunogenicity. DNA samples are collected from subjects who provide informed consent. These samples may be analyzed for genetic factors contributing to the subject's response to ABT-122, or other study treatment, in terms of pharmacokinetics, immunogenicity, tolerability and safety. Such genetic factors may include genes for drug metabolizing enzymes, drug transport proteins, genes within the target pathway, or other genes believed to be related to drug response. Some genes currently insufficiently characterized or unknown may be understood to be important at the time of analysis. The samples are analyzed as part of a multi-study assessment of genetic factors involved in the response to ABT-122 or drugs of this class. The samples may also be used for the development of diagnostic tests related to ABT-122 (or drugs of this class). The results of pharmacogenetic analyses may be reported with the study summary.

The use of NSAIDs (up to 125% the recommended dose), and cyclooxygenase (COX)-2 inhibitors are allowed during the study if patients are receiving stable prescribed doses for >4 weeks before the first study drug administration. Analgesic and anti-inflammatory agents, including NSAID and COX-2 inhibitors cannot be taken within 12 hours before efficacy evaluations. Narcotics are prohibited.

### Diagnosis and Main Criteria for Inclusion/Exclusion:

### Main Inclusion:

1. Adult male or female, 18 to 75 years of age
2. Diagnosis of RA based on the 2010 American College of Rheumatology (ACR)/European League against Rheumatism (EULAR) criteria
3. Disease duration of at least 6 months
4. ≧6 Swollen joints (based on 66 joint counts) at baseline
5. ≧6 Tender joints (based on 68 joint counts) at baseline
6. Inadequate response to MTX treatment defined as oral or parenteral treatment with an unchanged mode of application and stable prescribed MTX dose for at least 4 weeks prior to baseline of ≧15 mg/week or ≧10 mg/week for MTX intolerance and <the upper limit of the applicable approved local label.

### Additional Inclusion:

RA diagnosis for at least 3 months from date of first screening.

Documented positive rheumatoid factor (RF) or anti-cyclic citrullinated peptide (anti-CCP) antibody levels prior to or at screening.

If RF or anti-CCP tests are negative, then documented evidence of prior erosions or radiographs or CT/MRI of hands is an acceptable alternative for this criterion.

### Main Exclusion:

1. The subject has previous exposure to any biologic DMARD treatment for RA or former randomization in an anti-TNF trial (e.g., infliximab, etanercept, adalimumab, golimumab, certolizumab pegol, or TNF biosimilar).
2. The subject has previous exposure to any Jak Kinase Inhibitor treatment for RA or former randomization in a Jak-inhibitor trial (e.g., tofacitinib [Xeljanz®], GLPG0624, ABT494, VX-509, Baricitinib, or ASP015K).
3. Treatment with traditional DMARDs apart from MTX 12 weeks prior to Day 1 and for leflunomide 24 weeks (except for specific leflunomide wash-out procedure, i.e., 11 days with colestyramine or activated charcoal plus 30 days wash-out) prior to Day 1.
4. Stable prescribed dose of oral prednisone or prednisone equivalent >10 mg/day within the preceding 30 days of first dose of study drug.
5. Intra-articular or parenteral administration of corticosteroids in the preceding 4 weeks of first dose of study drug. Inhaled corticosteroids for stable medical conditions are allowed.
6. Laboratory values of the following at the Screening Visit:

Confirmed hemoglobin <9 g/dL for males and 8.5 g/dL for females
Absolute neutrophil count (ANC)<1500 μ/L

AST or ALT >1.5× the upper limit of normal (ULN) or bilirubin >3 mg/dL Serum creatinine >1.5× the ULN

### Alternative Exclusions

1. Subject has previous exposure to Humira®, other TNF inhibitors or other biological DMARDs.
2. Current treatment with traditional oral DMARDs (except for concomitant treatment with sulfasalazine and/or hydroxychloroquine in addition to MTX). Oral DMARDs must be washed out 5 times the mean terminal elimination half-life of a drug apart from MTX prior to Day 1.
Subject could have been exposed to prior JAK inhibitors so long as they have been off therapy for 5 half-lives.
   3. Stable prescribed dose of oral prednisone or prednisone equivalent >10 mg/day within the 30 days of first dose of study drug.
   4. Intra-articular or parenteral administration of corticosteroids in the preceding 4 weeks of first dose of study drug. Inhaled corticosteroids for stable medical conditions are allowed.
   5. Laboratory values of the following at the Screening Visit:
Confirmed hemoglobin <9 g/dL for males and <8.5 g/dL for females,
Absolute neutrophil count (ANC)<1500/L,
AST or ALT >1.5 Å˜the upper limit of normal (ULN) or bilirubin 3 mg/dL,
Serum creatinine >1.5 Δ˜the ULN,
Platelets <100,000 cells/mm³ (10⁹/L),
Clinically significant abnormal screening laboratory results as evaluated by the Investigator.

For all subjects, pharmacokinetic (PK) trough samples are collected at each specified visit beginning on Day 1 through Week 12. For 30% of subjects, in addition to trough PK samples at each visit, PK samples are collected on Day 1 and Week 12 at 2 hours and 72 to 120 hours post-dose. Statistical methods are used to determine the efficacy of ABT-122.

### Efficacy:

Thirty subjects per treatment group provide an estimate of the ACR20 response rate for the three ABT-122 dose groups (high dose, mid dose, and low dose) to establish proof of concept for ABT-122. Historical ACR-20 response rate for the reference arm, adalimumab 40 mg administered EOW, along with the ACR-20 response observed in this study are used to evaluate the relative efficacy of ABT-122 administered compared to adalimumab (40 mg) administered EOW.

### Pharmacokinetics:

ABT-122 plasma concentrations are listed for each subject by visit day and dose group. Ctroughs are summarized by visit day and dose group. The pharmacokinetic parameters such as Cmax, Tmax and AUC are estimated directly from the data in the 30% subjects who have more intensive PK sampling on Day 1 and Week 12 if appropriate. The PK data from all subjects are optionally analyzed using population approach. The relationship between ABT-122 exposure and clinical efficacy or safety response(s) is explored based on the data obtained.

### Additional Analysis/Data Based on Study Pharmacodynamic and Safety:

Data obtained in the study above are used to analyze parameters including pharmacodynamics and safety. The results of the studies described herein show that ABT-122 therapy according to the invention is effective as a treatment for treating RA in patients resistant to MTX treatment. ABT-122 protein therapy is used to treat RA in the subject suffering from any type of symptom/condition including, but not limited to, pain, joint inflammation, and joint damage. Therapeutic treatment can be provided to a subject by administering to the subject a combination (for example, a mixture, concurrent administration, or successive administration) of ABT-122 with MTX.

### Example 9

### Study M12-965—a Phase 2, Multicenter, Open-Label Extension (OLE) Study with ABT-122 in Rheumatoid Arthritis Subjects Who have Completed the Preceding M12-963 Phase 2 Randomized Controlled Trial (RCT)

The primary objective of this study is to assess the long-term safety and tolerability of ABT-122 in subjects with rheumatoid arthritis (RA) who have completed study M12-963 Phase 2 RCT described above. The secondary objectives include analyzing: the effect of continued dosing on anti-drug antibody (ADA) profiles for ABT-122; influence of ABT-122 dose on maintenance of efficacy, as assessed by American College of Rheumatology (ACR) response criteria and European League against Rheumatism (EULAR) remission criteria and the individual components of these measures; and longer term effects of ABT-122 on function, quality of life, fatigue and work instability.

The study population includes adult female and male RA subjects. At least about 80 subjects are enrolled in this OLE study. This is a 24-week open-label extension study to assess the safety and tolerability of ABT-122 in RA subjects who have completed the preceding study M12-963 RCT. Only those subjects who have met all of the specified inclusion and none of the exclusion criteria have an option to enter into the OLE study to receive ABT-122, as long as the subject is willing and the Investigator believes that continuing the therapy with ABT-122 is appropriate.

Subjects are on 120 mg ABT-122 every other week (EOW) in an open-label fashion with the possibility of an additional visit for one extra 120 mg dose based upon the loss of ACR20 response. The one time extra dose takes place anywhere between Weeks 12 through 20 of the study (within ±2 days of the regularly scheduled weekly visit). Subjects may down titrate once to 60 mg EOW due to safety and tolerability concerns based on the investigator's medical judgment anywhere between Weeks 2 through 20 of this study and remain on that 60 mg dose

EOW for the remainder of this study. If the subject down titrates to 60 mg EOW for safety or tolerability reason, that subject cannot receive the extra 120 mg dose following the down titration to the 60 mg EOW regimen. Subjects are only allowed one down titration of the dose and if they experience any additional safety or tolerability issues, they should be permanently discontinued.

The specifics for the study are described below in Table 19.

TABLE 19
Study Information and Treatments
Administered for Study M12-965

| Investiga- | ABT-122 100 mg Powder |
|---|---|
| tional Product | for solution for Injection Vial |
| Dosage Form | Lyophilized powder for injection in vials |
| Formulation | ABT-122, Sucrose, Histidine, Polysorbate 80, Water for |
| | injections, Hydrochloric Acid added as necessary to |
| | adjust pH |
| Strength | 100 mg/mL when reconstituted with 1.2 mL of sterile |
| (mg) | water for injection |
| Mode of | Subcutaneous injection |
| Administration | |

Approximately 80 subjects, who have completed Study M12-963 RCT are enrolled in this OLE study. Only those subjects who have met all of the specified inclusion and none of the exclusion criteria in described herein have an option to enter into this OLE study to receive ABT-122, as long as the subject is willing and the Investigator believes that continuing the therapy with ABT-122 is appropriate.

### Inclusion Criteria

1. Subjects who have completed the preceding Study M12-963 (ABT-122) RCT study and have not developed any discontinuation criteria, as defined in the criteria section for Study M12-963.
2. If female, subject must meet one of the following criteria: Postmenopausal (defined as no menses for at least 1 year); Surgically sterile (bilateral tubal ligation, bilateral oophorectomy or hysterectomy); Practicing appropriate birth control, from the time of enrollment in this study until at least 150 days after the last dose of study drug. Females who have undergone tubal ligation are required to agree to use a second form of contraception for the same period of time.
3. Male who agrees to follow protocol-specified pregnancy avoidance measures, including refraining from donating sperm, for up to 150 days post last dose of study drug.
4. Subjects must voluntarily sign and date an informed consent, approved by an Independent Ethics Committee (IEC)/Institutional Review Board (IRB), prior to the initiation of any screening or study-specific procedures.

5. Subject is judged to be in good health as determined by the Investigator based on the results of medical history, physical examination and laboratory profile performed.

### Exclusion Criteria

1. Pregnant or breastfeeding female.
2. Ongoing infections at Day 1 (Week 0) that have NOT been successfully treated within 14 days.
3. Anticipated requirement or receipt of any live vaccine during study participation including up to 120 days after the last dose of study drug.
4. Current enrollment in another investigational study; with the exception of Study M12-963, which is required.
5. Consideration by the Investigator, for any reason, that the subject is an unsuitable candidate to receive ABT-122.

### Prior and Concomitant Therapy

Any medication or live/attenuated vaccines (including over-the-counter or prescription medicines, vitamins and/or herbal supplements) that the subject is receiving during the study, must be recorded along with the reason for use, date(s) of administration including start and end dates, and dosage information including dose, route and frequency on the appropriate eCRF.

Subjects should remain on a stable dose of MTX throughout Study M12-965. However, if a subject experiences MTX-induced toxicity, MTX dose can be reduced or discontinued only after consulting with the study designated physician.

Subjects are required to use folic acid (or equivalent) during the study, with the dose and regimen chosen per investigator's judgment. If subject is already taking folic acid or equivalent, the dose should remain stable throughout study participation.

Stable doses of non-steroidal anti-inflammatory drugs (NSAIDs), cyclooxygenase (COX) 2 inhibitors, acetaminophen, or oral corticosteroids (equivalent to prednisone <10 mg) are allowed throughout the study. Subjects taking NSAIDs should be advised not to take NSAIDs at least 4-6 hours prior to study visit. Substitution with another NSAID is permitted.

Narcotics are prohibited except combinations with acetaminophen. Narcotics in combination with acetaminophen are prohibited to be taken within 24 hours prior to joint assessments at study visits. Sulfasalazine and/or hydroxychloroquine are allowed so long as they are used according to the local label and are used in addition to MTX.

Stable doses of oral corticosteroids (equivalent to prednisone <10 mg) are allowed as long as maintained at a stable dose throughout the study. If the subject is taking vitamin D supplements, the subject should continue on stable doses throughout the duration of the trial In addition for subjects aged ≦30 with a reported malignancy adverse event, prior exposure to, or current use of, antineoplastics, or other drugs which have a risk of malignancy as stated in their label and other relevant dosing information to estimate total exposure are collected in the source documents and appropriate eCRF pages. At the time of the reported malignancy adverse event, sites are asked if any of the prior and concomitant medications contributed to the event. Any medications used prior to the study are captured on the appropriate eCRF. Information on the reason for use, date(s) of administration including start and end dates, highest maintained dose, dosage information including dose, route and frequency, and reason for stopping the medication are collected in the source documents and appropriate eCRF pages.

Medications used to treat suspected hypersensitivity reaction or other post-dose systemic reaction are captured as concomitant therapy. The study designated physician should be contacted if there are any questions regarding concomitant or prior therapy or therapies.

Prohibited Therapy

The following medications are prohibited throughout the study: Oral Disease modifying antirheumatic drugs (DMARDs) (except MTX, sulfasalazine and/or hydroxychloroquine together with MTX); all biologic therapies; high potency opiates; live vaccines; anti-retroviral therapy; any experimental therapies, other than ABT-122.

### Efficacy and Safety Assessments/Variables and Efficacy and Safety Measurements Assessed and Flow Chart

All subjects must meet the study selection criteria outlined herein in order to be randomized in to the study. The study is 24 weeks in duration with study visits EOW (FIG. 9). The treatment period for OLE is defined as Day 1 (Week 0), and ends at Week 24. The Last Visit (Week 12) of Study M12-963, is the first visit Day 1 (Week 0) for the Study M12-965 OLE study. Subjects who meet all the inclusion criteria and none of the exclusion criteria described herein are eligible to enroll into this study. Subjects have up to 14 days between RCT Week 12 visit and the first visit of this OLE study. Subjects that are not enrolled within the 14 days are not eligible to participate in this OLE study. Subjects visit the study site at Day 1 (Week 0), Weeks 2, 4, 6, 8, 10, 12, 14, 16, 18, 20, 22, 24 or if they terminate early from the study. A ±3-day window is permitted around scheduled study visits. The first dose of study drug is given at Day 1 (Week 0) in this OLE study and the last dose of study drug is given at the Week 22 visit.

Subjects may discontinue study drug treatment at any time during study participation. Subjects who end study participation early have a Premature Discontinuation Visit and complete the procedures outlined for Premature Discontinuation Visit as soon as possible after the last dose of study drug and preferably prior to the administration of any new therapies.

### Treatments Administered

All subjects are on ABT-122 120 mg EOW in an open-label fashion with the possibility of an extra 120 mg dose based upon the loss of ACR20 response. This one time extra dose can take place anywhere between Weeks 12 through 20. Subjects may down titrate once to 60 mg EOW due to safety and/or tolerability concerns based on the investigator's medical judgment anywhere between Weeks 2 through 20 and remain on that 60 mg dose EOW for the remainder of the study. If the subject down titrates to 60 mg EOW for safety and tolerability and has not already received the single extra 120 mg dose, they cannot have the extra dose following down titration to 60 mg EOW. Subjects continue their weekly stable dose of MTX throughout the study. The areas to avoid for SC injections include: any blood vessels, thickening or tende mess of skin, scars, fibrous tissue, lesions, stretch marks, bruises, redness, nevi, or other skin imperfections. Injection sites should be at least 1 inch apart and at least 2 inches from the navel.

Blood Samples for Assays of ABT-122 and ABT-122 sADA/nADA

Blood samples for the ABT-122 PK, sADA and nADA assays are centrifuged within

30-60 minutes of collection to separate the serum. At time points where ABT-122 PK, sADA and nADA need to be collected the total volume of serum derived from the 10 mL draw are equally split using plastic pipettes over 6 (approximately 0.75 mL per vial) screw-capped polypropylene cryotubes. At screening time points the total volume of serum derived from the 3 mL draw are equally split using plastic pipettes over 2 (approximately 0.75 mL per vial) screw-capped polypropylene cryotubes. The tubes are labeled and the serum samples are placed in the freezer within 2 hours after collection and maintained at −20° C. or colder until shipped. Samples for the nADA assay are banked and analyzed upon request. The nADA samples, sADA, and PK samples collected may also be used for assay development.

### Blood Samples for ABT-122 Pharmacokinetic Assay

Blood samples for ABT-122 assay are collected by venipuncture into appropriately labeled evacuated 10 mL serum collection tubes without gel separators. Blood is allowed to clot for at least 30 to 60 minutes at room temperature before centrifgation. Blood samples for the PK assay are collected. Thirteen samples are collected per subject for pharmacokinetic analysis. An extra pre-dose PK sample is collected in the case of an unscheduled visit for an additional 120 mg ABT-122 dose.

### Blood Samples for ADA Assay

The serum samples for the screening (sADA) and neutralizing (nADA) assays are taken from the serum collected from the 10 mL venipuncture draw for ABT-122 (PK). Blood samples for the ADA assays are collected.

Six samples are collected per subject for ADA analysis during the dosing phase of the study. One additional sample is collected 70 days after last dose of study drug administration per subject. The total number of blood samples planned for ADA analysis is 7. An extra pre-dose ADA sample iscollected in case of an unscheduled visit for an additional 120 mg ABT-122 dose and/or at the dosing visit where ABT-122 dose is to be tapered to 60 mg EOW for safety reasons.

### Efficacy Variables

An objective of this study is to evaluate the safety and tolerability of ABT-122 in RA subjects who have completed Study M12-963 Phase 2 RCT. ACR20/50/70 response rate by visit is summarized. Change from baseline for ACR individual components, DAS28[hsCRP] and Patient Reported Outcomes including FACIT, RA-WIS, SF-36, proportion of subjects achieving Low Disease Activity (LDA) or Clinical Remission (CR) based on DAS28[hsCRP] and CDAI criteria are summarized by visit.

### Safety Variables

Incidence of AEs, changes in vital signs, physical examination results, and clinical laboratory data are assessed throughout the study.

### Pharmacokinetic Variables

Serum ABT-122 concentrations are determined every 2 weeks during the 24-week treatment period. PK and ADA data may be combined with data from other studies and analyzed using a mixed-effects modeling approach. This analysis estimates the population central value and the empirical Bayesian estimates of the individual values for ABT-122 apparent clearance (Cl/F) and volume of distribution (Vss/F). Additional parameters may be estimated if useful in the interpretation of the data. Data from this study may be combined with data from other ABT-122 studies for the population analysis.

Pharmacodynamic and mRNA Biomarkers

Blood samples are collected to assess the mechanism of action of ABT-122.

### Disease Response Biomarkers

Subjects have additional blood and urine samples collected to assess disease response. Samples are analyzed for measurement of non-genetic markers related to disease activity/prognosis of RA, autoimmunity/inflammation, and/or response to anti-RA medications, including ABT-122 or drug of this class.

### Clinical Assessments

To explore the potential disease response signals, the following clinical assessments are obtained: swollen joint count, tender joint count, physician's global assessment of disease activity using a visual analog scale (VAS), patient's global assessment of disease activity using VAS, patient's assessment of pain using VAS. As well as the following patient reported outcomes questionnaires HAQ-DI, RA-WIS, FACIT-F, and SF-36v2.

### Discontinuation of Individual Subjects

A subject may withdraw from the study at any time. An investigator may discontinue any subject's participation for any reason, including an AE, safety concerns or failure to comply with the protocol. Subjects are withdrawn from the study if any of the following occur: clinically significant confirmed abnormal laboratory results or AEs, which rule out continuation of the study medication, as determined by the Investigator and the study designated physician; a subject experiences a SAE for which there is no clear alternative explanation (e.g., the subject is a victim of a motor vehicle accident); a subject experiences a moderate/grade 2 or above non-serious AE of a systemic hypersensitivity reaction, for which there is no clear alternative explanation; a subject experiences a moderate/grade 2 AE of vasculitis for which there is no clear alternative explanation; the Investigator believes it is in the best interest of the subject; the subject requests withdrawal from the study; inclusion and exclusion criteria violation was noted after the subject started study drug, when continuation of the study drug would place the subject at risk as determined by the study designated physician; introduction of prohibited medications or dosages when continuation of the study drug would place the subject at risk as determined by the e study designated physician; the subject becomes pregnant while on study medication; subject has known dysplasia of the gastrointestinal tract (a colonoscopy is not required to enter the study) or malignancy, except for localized non-melanoma skin cancer. Discontinuation for carcinoma in-situ of the cervix is at the discretion of the Investigator; subject is diagnosed with lupus like syndrome, multiple sclerosis or demyelinating disease (including myelitis); subject is significantly non-compliant with study procedures which would put the subject at risk for continued participation in the trial in consultation with the study designated physician; subject experiences severe, grade 3 or greater, or life-threatening injection site reaction (ISR) which includes prolonged induration, superficial ulceration and includes thrombosis or major ulceration or necrosis requiring surgery; and a subject has a confirmed platelet count <50,000 cells/mm³

If, during the course of study drug administration, the subject prematurely discontinues study drug use, the procedures outlined for the PD visit must be completed within 2 weeks of the last dose of study drug, and preferably prior to the initiation of another therapy. However, these procedures should not interfere with the initiation of any new treatments or therapeutic modalities that the Investigator feels are necessary to treat the subject's condition. For subjects that prematurely discontinue, study drug is not given at the premature discontinuation visit.

Following discontinuation of the study drug, the subjects are treated in accordance with the Investigator's best clinical judgment.

A final visit takes place for all subjects 70 days after the last study drug administration to determine the status of any ongoing AEs/SAEs or the occurrence of any new AEs/SAEs.

### Criteria for Evaluation:

### Efficacy:

ACR20/50/70 response rate at all visits is summarized. Change from baseline for ACR individual components, DAS28[hsCRP] and Patient Reported Outcomes including FACIT, RA-WIS, SF-36, proportion of subjects achieving Low Disease Activity (LDA) or Clinical Remission (CR) based on Disease activity score 28 (DAS28)[hsCRP] and Clinical Disease Activity Index (CDAI) criteria are summarized by visit.

### Pharmacokinetic and Immunogenicity:

ABT-122 serum concentrations are determined A mixed-effects modeling approach are used to estimate the population central value and the empirical Bayesian estimates of the individual values for ABT-122 apparent clearance (Cl/F) and volume of distribution (Vss/F). Additional parameters may be estimated if useful in the interpretation of the data. Pharmacokinetic data from this study may be combined with data from other ABT-122 studies for the population pharmacokinetic analysis. Multiple measurements of ADA are collected for each patient during the treatment period and the follow-up visit. The percentage of subject with ADA are calculated. As appropriate, the effect of ADA on ABT-122 pharmacokinetics and efficacy are explored.

### Pharmacodynamics:

Multiple panels of serum/plasma biomarkers are collected for exploratory purposes. The samples are archived for potential testing at a later date. Data from these exploratory pharmacodynamic endpoints may not be part of the clinical study report. Pharmacokinetic/pharmacodynamics relationship is explored across several clinical laboratory endpoints.

### Disease Response Biomarkers

Blood and urine samples are collected. The panel may include, but is not limited to: Matrix metalloproteinase-mediated C-reactive protein (CRPM), Matrix metalloproteinase 3 (MMP-3), Matrix metalloproteinase-mediated degradation of type I collagen (C1M), Matrix metalloproteinase-mediated degradation of type II collagen (C2M), Matrix metalloproteinase-mediated degradation of type III collagen (C3M), C-terminal telopeptide type I collagen (CTX-I), C-terminal telopeptide type II collagen (CTX-II), Osteocalcin and Citrullinated and matrix metalloproteinase-degraded vimentin (VICM)

### Safety:

Safety evaluations include adverse event (AE) monitoring, physical examinations, vital sign measurements, electrocardiogram, and clinical laboratory testing (hematology, chemistry, and urinalysis) as a measure of safety and tolerability. Toxicity management guidelines are provided within the protocol. This study uses a safety review committee that is independent of the study team.

### Statistical Methods:

### Efficacy:

For analyses purposes, baseline data for each subject is the data collected at the baseline visit of the RCT, immediately prior to starting treatment with double-blind medication. The response rates of ACR20/50/70 are summarized with 95% confidence intervals by visit. Change from Day 1 (Week 0) is summarized with descriptive statistics for tender joint count (TJC), swollen joint count (SJC), Patient's Assessment of Pain, Patient's Global Assessment of Disease Activity, Physician's Global Assessment of Disease Activity, HAQ-DI, hsCRP, DAS28[CRP], CDAI by visit. The proportion of subjects achieving LDA (2.6<DAS28[CRP]<3.2 or 2.8<CDAI≦10) or CR (DAS28[CRP]<2.6 or CDAI≦2.8), and the proportion of subjects achieving CR (DAS28[CRP]<2.6 or CDAI≦2.8) is summarized with 95% confidence intervals by visit.

### Safety:

All subjects who receive at least one dose of ABT-122 during the OLE study are included in the safety analysis. Incidence of adverse events, serious adverse events (SAE), premature discontinuation, and changes from Day 1 (Week 0) in vital signs, physical examination results and clinical laboratory values are analyzed by visit. Treatment-emergent AEs are tabulated by system organ class and by MedDRA preferred term. Mean change from baseline for laboratory and vital signs data are summarized by visit. For analyses purposes, baseline for vital signs, physical examination results, and clinical laboratory results for subjects is the data collected at the visit immediately prior to starting treatment with double-blind medication.

### Example 10

### Physical, Chemical, and Pharmaceutical Properties and Formulation of ABBV-257, a DVD-Ig Binding Protein

The dual binding and/or neutralization of TNF and IL-17 may provide superior efficacy to the current standard of care treatments for autoimmune and inflammatory diseases. Shown in Table 20 below are amino acid sequences for ABBV-257, a DVD-Ig binding protein having heavy chain and light chain domains comprising humanized and affinity matured variable domain sequences from mouse anti-TNF and anti-IL-17 antibodies.

TABLE 20
Affinity Matured DVD-Ig ™ Protein Heavy
Variable Domain And Light Variable Domain
Of Anti-IL-17/TNF DVD-Ig ™ Protein ABBV-257

| | | |
|---|---|---|
| DVD HEAVY | SEQ ID | EVQLVQSGAEVKKPGASVKV |
| VARIABLE | NO.: 69 | SCKASGYTFANYGIIWVRQA |
| HMAK199-1-GS10- | | PGQGLEWMGWINTYTGKPTY |
| H10F7-M11 DVD | | AQKFQGRVTMTTDTSTSTAY |
| | | MELSSLRSEDTAVYYCARKL |
| | | FTTMDVTDNAMDYWGQGTTV |
| | | TVSSGGGGSGGGGSEVQLVQ |
| | | SGAEVKKPGSSVKVSCKASG |
| | | YTFTDYEIHWVRQAPGQGLE |
| | | WMGVNDPESGGTFYNQKFDG |
| | | RVTLTADESTSTAYMELSSL |
| | | RSEDTAVYYCTRYSKWDSFD |
| | | GMDYWGQGTTVTVSS |
| HMAK199-1VH | SEQ ID | EVQLVQSGAEVKKPGASVKV |
| | NO. 70 | SCKASGYTFANYGIIWVRQA |
| | | PGQGLEWMGWINTYTGKPTY |
| | | AQKFQGRVTMTTDTSTSTAY |
| | | MELSSLRSEDTAVYYCARKL |
| | | FTTMDVTDNAMDYWGQGTTV |
| | | TVSS |
| LINKER | SEQ ID | GGGGSGGGGS |
| | NO.: 71 | |
| H10F7-M11 VH | SEQ ID | EVQLVQSGAEVKKPGSSVKV |
| | NO.: 72 | SCKASGYTFTDYEIHWVRQA |
| | | PGQGLEWMGVNDPESGGTFY |
| | | NQKFDGRVTLTADESTSTAY |
| | | MELSSLRSEDTAVYYCTRYS |
| | | KWDSFDGMDYWGQGTTVTVS |
| | | S |
| CH CG1234, 235 | SEQ ID | ASTKGPSVFPLAPSSKSTSG |
| MUT Z NONA | NO.: 73 | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| DVD LIGHT | SEQ ID | DIQMTQSPSSLSASVGDRVT |
| VARIABLE | NO.: 74 | ITCRASQDISQYLNWYQQKP |
| HMAK199-1-GS10- | | GKAPKLLIYYTSRLQSGVPS |
| H10F7-M11DVD | | RFSGSGSGTDFTLTISSLQP |
| | | EDFATYFCQQGNTWPPTFGQ |
| | | GTKLEIKRGGSGGGGSGDIQ |
| | | MTQSPSSLSASVGDRVTITC |
| | | RASSGIISYIDWFQQKPGKA |
| | | PKRLIYATFDLASGVPSRFS |
| | | GSGSGTDYTLTISSLQPEDF |
| | | ATYYCRQVGSYPETFGQGTK |
| | | LEIKR |
| HMAK199-1 VL | SEQ ID | DIQMTQSPSSLSASVGDRVT |
| | NO.: 75 | ITCRASQDISQYLNWYQQKP |
| | | GKAPKLLIYYTSRLQSGVPS |
| | | RFSGSGSGTDFTLTISSLQP |
| | | EDFATYFCQQGNTWPPTFGQ |
| | | GTKLEIKR |
| LINKER | SEQ ID | GGSGGGGSG |
| | NO.: 76 | |
| H10F7-M11VL | SEQ ID | DIQMTQSPSSLSASVGDRVT |
| | NO. 77 | ITCRASSGIISYIDWFQQKP |
| | | GKAPKRLIYATFDLASGVPS |
| | | RFSGSGSGTDYTLTISSLQP |
| | | EDFATYYCRQVGSYPETFGQ |
| | | GTKLEIKR |
| CL | SEQ ID | TVAAPSVFIFPPSDEQLKSG |
| | NO.: 78 | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

*Note that the component CDRS of the VH and VL binders are in bold

ABBV-257 is a recombinant DVD-Ig comprised of 2 identical×light chains and 2 identical IgG1 heavy chains covalently attached through a full complement of inter- and intra-molecular disulfide bonds. The disulfide linkage pattern is structurally similar to that of natural IgG1 antibodies. The heavy chain is post-translationally modified by addition of N-linked glycans to the heavy chain at the same asparagine location commonly modified on IgG1 antibodies. The major glycans are fucosylated biantennary oligosaccharides containing 0, 1, or 2 galactose residues. Each light chain and heavy chain contains two variable domains connected in tandem by flexible glycine-serine peptide linker regions enabling dual specificity capable of binding both IL-17 and TNF in a tetravalent manner. Except for these linkers, the heavy chain and light chain variable and constant regions of ABBV-257 have humanized or fully human amino acid sequences. ABBV-257 has a molecular weight of 202 kDa and solubility of about 50 mg/mL at a minimum in formulation buffer. It is a lyophilisate powder at 50 mg/mL after reconstitution in histidine, sucrose, polysorbate-80. The drug product (ABBV-257 powder for solution for injection, 50 mg/mL, in vials) was stored refrigerated at 2° to 8° C. and protected from light. The drug product was not frozen. ABBV-257 is a high-affinity human recombinant DVD-Ig with an IgG1 constant region and a x light chain. The human IgG1 constant region in ABBV-257 contains 2 mutations (L234A, L235A) in the lower hinge region that significantly reduce binding to Fcγ receptors, and 2 mutations (T250Q and M428L) that enhance its binding to neonatal Fc receptor (FcRn) at intracellular acidic pH to increase recycling and extend serum half-life of the molecule. 1 ABBV-257 selectively neutralizes human TNF and IL-17A and does not recognize a panel of other cytokines in the TNF or IL-17 families. The in vitro pharmacologic properties of the clinical candidate ABBV-257, as well as the efficacy data with surrogate anti-mouse TNF and IL-17 antibodies, are described in Examples herein.

### Example 11

### In Vitro Pharmacology of ABBV-257

The kinetic binding of ABBV-257 to TNF and IL-17 were determined using Biacore® surface plasmon resonance technology. The apparent association rate (ka) and dissociation rate (kd) were derived and used to calculate the overall equilibrium dissociation constant (KD) for the interaction. The results from several experiments indicate that ABBV-257 has very high affinity for both TNF and IL-17 as shown in Table 21.

TABLE 21
Binding Affinity of ABBV-257 for Recombinant TNF and IL-17

| Measure- | TNF | IL-17 A/A | IL-17A/F | |
|---|---|---|---|---|
| ment | Mean ± SD | Mean ± SD | Mean ± SD | N |
| ka | 3.3 ± 1.2 × 10⁶ | 1.1 ± 0.12 × 10⁶ | 3.4 ± 0.2 × 10⁵ | 3 |
| (M⁻¹s⁻¹) | | | | |
| kd (s⁻¹) | 1.6 ± 0.7 × 10⁵ | 3.0 ± 1.8 × 10⁻⁶ | 4.8 ± 0.4 × 10⁻⁶ | 3 |
| K_{D} (pM) | 4.9 ± 0.5 | 3.0 ± 2.1 | 14 ± 2 | 3 |

N = number of experiments

### Example 12

### In-Vitro Potency for ABBV-257 for Human TNF and IL-17

Data show that ABBV-257 fully neutralized human TNF and IL-17 bioactivity. The in vitro neutralization potency (inhibitory concentration 50%; IC50) of ABBV-257 was determined by measuring the amount of ABBV-257 required to inhibit 50% of either the TNF-induced lethality of L929 cells or the IL-17 dependent induction of IL-6 in fibroblasts. ABBV-257 neutralized both the A/A and A/F isoforms of IL-17, as shown in Table 22.

TABLE 22
In-Vitro Potency of ABBV-257 for Human TNF and IL-17

| | Mean IC50 ± SD (pM) | No. of Experiments |
|---|---|---|
| TNF | 25 ± 8 | 3 |
| IL-17 A/A | 26 ± 6 | 3 |
| IL-17 A/F | 110 ± 10 | 3 |

### Example 13

### Specificity of ABBV-257

The specificity of ABBV-257 for TNF and IL-17A was determined by assessing its binding to cytokines in the IL-17 and TNF families by direct enzyme-linked immunosorbent assay (ELISA). ABBV-257 bound to IL-17A and IL-17A/F heterodimer as expected but did not bind to IL-17B, IL-17C, IL-17D, or IL-17E (IL-25). Similarly, ABBV-257 bound to TNF, but not to the family members lymphotoxin a, 4-1BB ligand, LIGHT, APRIL, BAFF, OX40 ligand, CD30 ligand, TL1A, CD40 ligand, EDA-A2, RANK ligand, Fas ligand, TWEAK, and GITR ligand.

### Example 14

### In Vitro Species Cross Reactivity of ABBV-257

The cross reactivity of ABBV-257 to recombinant TNF (rTNF) and IL-17 of other species was assessed by determining the IC50 in an in vitro neutralization assay, as well as by determining the K_{D} using Biacore®, analysis. ABBV-257 neutralized monkey TNF and IL-17 with similar IC₅₀ compared to human (Table 23). In contrast, the IC₅₀ for rodent and rabbit IL-17 was markedly increased compared to human and did not neutralize rodent or rabbit TNF. Consistent with these findings, the KD of ABBV-257 for monkey TNF and IL-17 was similar to those in humans, and was increased for rodent and rabbit IL-17, correlating with the increased IC₅₀ in the bioassay (Table 24). No binding to rodent or rabbit TNF was detected even at very higher concentrations of rTNF. The full neutralization of monkey rTNF and rIL-17 in the bioassays supported the selection of cynomolgus monkey as a pharmacologically appropriate species for toxicological testing of ABBV-257. In addition, the lack of neutralization of rodent and rabbit TNF precluded the use of these species for toxicological studies.

TABLE 23
In-Vitro Potency of ABBV-257 for
Monkey and Rodent TNF and IL-17

| Species | IL-17 | TNF | N |
|---|---|---|---|
| Human | 26 ± 6 | 25 ± 8 | 3 |
| Monkey (Rhesus/Cynomolgus) | 32 ± 4 | 25 ± 7 | 3 |
| Mouse | 239 ± 67 | NI | 3 |
| Rat | 135 ± 24 | NI | 3 |
| Rabbit | 11400 ± 300 | NI | 3 |

N = number of experiments;
NI = not inhibited (at concentrations up to 1 μM)

TABLE 24
Binding Affinity of ABBV-257 for
Monkey and Rodent TNF and IL-17

| Species | IL-17 | TNF | N |
|---|---|---|---|
| Human | 3.0 ± 2.1 | 4.9 ± 0.5 | 3 |
| Monkey (Rhesus/Cynomolgus) | 11 ± 4.1 | 24 ± 4 | 3 |
| Mouse | 72 ± 2.9 | NB | 3 |
| Rat | <35 | NB | 3 |
| Rabbit | 3300 ± 100 | NB | 3 |

N = number of experiments;
NB = No binding (at concentrations up to 500 nM of rTNF).

### Example 15

### Functional Properties of Fc Domain of ABBV-257 and Activation of Immune Cells

In vitro assays were performed to characterize the Fc-effector function and the potential of ABBV-257 to activate immune cells. The Fc isotype of ABBV-257 is a human IgG1. The Fc region has been inactivated with regards to FcγR binding utilizing mutation of amino acids L240A L241A that reduce binding to Fcg receptors and C1q (Hezareh et al. (2002) J. Virol. 75(24):12161-12168 and Wines et al. (2000) J. Immunol. 164(10):5313-5318). As shown in Table 25, ABBV-257 significantly reduced binding to FcγR1, Ha (both 131H and R variants), IIb, and IIIa (158 H and V variants), which predicts a decreased ability to activate immune cells through antibody-dependent cell-mediated cytotoxicity.

ABBV-257 also demonstrated a decreased ability to bind complement component C1q. ABBV-257 DVD-Ig binding protein contains 2 mutations in the constant regions CH2 (amino acid glutamine; abbreviated as Q) and CH3 (leucine, abbreviated as L) that increase its binding to FcRn at the lower pH found in the endosomal compartment. Mutations in ABBV-257 DVD-Ig binding protein extend the serum half-life of ABBV-257.

TABLE 25
Fc Binding Characteristics of ABBV-257

| Function | Variant | Assay | Result |
|---|---|---|---|
| Fcγ RI binding | NA | Biacore | No significant binding |
| Fcγ RIIa | 131H | Biacore | No significant binding |
| Fcγ RIIa | 131R | Biacore | No significant binding |
| Fcγ RIIb | NA | Biacore | No significant binding |
| Fcγ RIIIa | 158F | Biacore | No significant binding |
| Fcγ RIIIa | 158V | Biacore | Lower than IgG1 control |
| | | | antibody |
| FcR_{N} | NA | Biacore | Increased binding |
| C1q binding | NA | ELISA | |

NA = not applicable

### Example 16

### Human Peripheral Blood Cell Assay of ABBV-257

The ability of ABBV-257 to bind or activate cellular components of human blood was assessed in vitro utilizing peripheral blood from healthy donors. The interaction of ABBV-257 with human peripheral blood was analyzed by flow cytometry from three human blood donors utilizing fluorescently tagged ABBV-257 (fluorescein isothiocyanate [FITC]; ABBV-257-FITC) binding protein. These data demonstrated minimal binding of ABBV-257-FITC to human peripheral blood cells. ABBV-257 binding protein did not cause any platelet aggregation following incubation at 100 ηg/mL. It was observed that ABBV-257 did not induce production of cytokines from peripheral blood cells in an ex vivo cytokine release assay in which whole blood from three human blood donors was incubated with plate-bound compound for 48 hours at 37° C. There was no statistically significant secretion of IL-1β, IL-1ra, IL-6, IL-8, or TNF-α compared to a negative control antibody.

### Example 17

### ABBV-257 Pharmacokinetic Parameters after a Single Dose

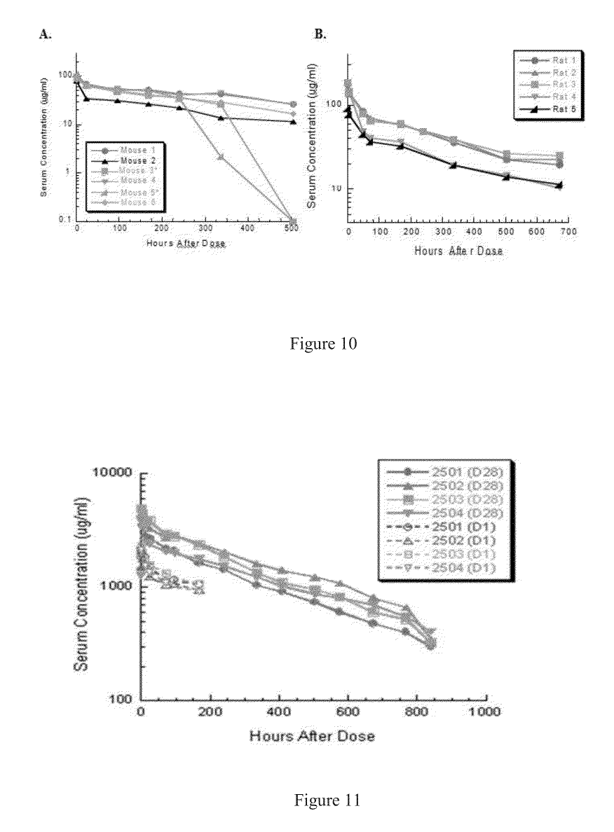

The pharmacokinetic profile of ABBV-257 following single IV doses in mouse and rat was characterized by low clearance values (0.2 and 0.15 mL/hr·kg in mice and rats, respectively), with low volumes of distribution (Vss=83.3 and 79.0 mL/kg in mice and rats, respectively). The terminal half-life in mice and rats was 12.9 and 17.5 days, respectively (Table 26). Serum exposure was maintained in 4/6 mice (FIG. 10 panel A) and in 5/5 rats (FIG. 10 panel B).

TABLE 26
ABBV-257 Pharmacokinetics Following a Single Intravenous
Dose in CD-1 Mice and Sprague-Dawley Rat

| | | | Mean (SD) | | | | |
|---|---|---|---|---|---|---|---|
| | Dose | t_{1/2} | V_{SS} | AUC_{0-inf} | CL | MRT | |
| Species | (mg/kg | (day) | (mL/kg) | (mg · hr/mL) | (mL/hr · kg) | (days) | n |
| Mouse | 5 | 12.9 | 83.3 (24.9) | 29.1 (10.7) | 0.20 (0.09) | 18.8 (24.9) | 4 |
| Rat | 5 | 17.5 | 79.0 (23.4) | 36.8 (11.8) | 0.15 (0.05) | 22.7 (4.3) | 5 |

t½ = terminal half-life;
Vss = volume of distribution at steady state;
AUC0-inf = area under the concentration-time curve from time zero up to infinite time;
CL = clearance;
MRT = mean residence time

In cynomolgus monkey, ABBV-257 serum exposures were not maintained throughout the study (up to 35 days) after a single 20 mg/kg dose of ABBV-257. Loss of exposure was observed after Day 14, may be because of the development of A DA

### Example 18

### Human Peripheral Blood Cell Assay of ABBV-257

ABBV-257 DVD-Ig binding protein was administered weekly (4 doses total) via IV infusion to female cynomolgus monkeys at a dose (100 mg/kg), followed by a 5-week washout period (n=4 per group). The terminal half-life observed after the fourth dose was 13.0 days (FIG. 11).

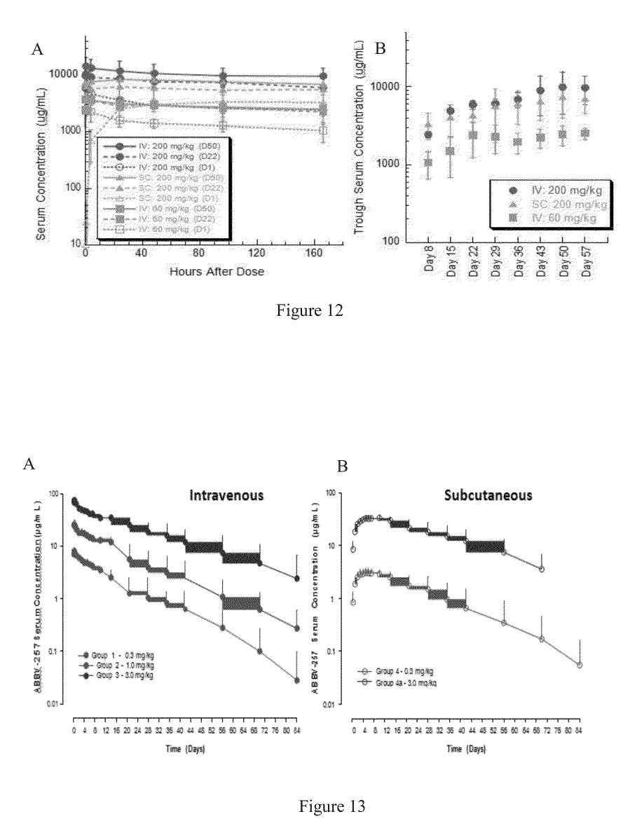

In the multiple dose Good Laboratory Practice (GLP) toxicity study, 2 groups of cynomolgus monkeys received 60 and 200 mg/kg doses of ABBV-257, administered as an intravenous bolus injection once per week for eight consecutive weeks. A third treatment group received a 200 mg/kg SC dose of ABBV-257 once weekly for eight consecutive weeks. Each treatment group contained four female and four male animals. The AUC and maximum concentration (Cmax) values increased in a dose-related fashion (FIG. 12; Table 27). Serum concentrations and AUC values for ABBV-257 did not appear to exhibit any sex-specific differences. The average of all AUC values in the 200 mg/kg SC dose group reached approximately 83% of the AUC values in the corresponding IV dose group. Peak plasma concentrations were noted 78 hours after the SC dose (average of Days 1, 22, and 50). Accumulation of ABBV-257 throughout the different dose groups was approximately a factor of 3, as indicated by an increase of the trough concentration (Ctrough) levels between Day 8 and Day 57.

TABLE 27
ABBV-257 Toxicokinetic Parameters After Intravenous
and Subcutaneous Injection in Cynomolgus Monkey
Following 8 Weeks of Once Weekly Dosing

| | ABBV-257 Dose (mg/kg) | | |
|---|---|---|---|
| Toxicokinetic | 60 IV | 200 IV | 200 SC |

| | |
|---|---|
| Parameter | Mean (SD) |

Day 1

| | | | |
|---|---|---|---|
| Number animals/group | 8 | 8 | 8 |
| Cₘₐₓ (mg/mL) | 2.49 (0.668) | 6.05 (2.44) | 3.68 (1.02) |
| Cₘₐₓ/D | 0.042 (0.011) | 0.030 (0.012) | 0.0184 (0.005) |
| (mg/mL/mg/kg) | | | |
| Tₘₐₓ (hr) | N/A | N/A | 116.3 (44.3) |
| AUC | 230 (50.8) | 498 (53.8) | 494 (146) |
| (mg · hr/mL) | | | |
| AUC/D | 3.84 (0.848) | 2.49 (0.27) | 2.47 (0.73) |
| (mg · hr/mL/mg/kg) | | | |

Day 22

| | | | |
|---|---|---|---|
| Number animals/group | 6^{a} | 7 | 8 |
| Cₘₐₓ (mg/mL) | 4.01 (0.782) | 10.9 (1.68) | 7.43 (2.74) |
| Cₘₐₓ/D | 0.067 (0.013) | 0.055 (0.008) | 0.037 (0.014) |
| (mg/mL/mg/kg) | | | |
| Tₘₐₓ (hr) | N/A | N/A | 92.3 (65.0) |
| AUC | 461 (95.5) | 1270 (175) | 971 (586) |
| (mg · hr/mL) | | | |
| AUC/D | 7.68 (1.6) | 6.33 (0.875) | 4.85 (2.93) |
| (mg · hr/mL/mg/kg) | | | |

Day 50

| | | | |
|---|---|---|---|
| Number animals/group | 6^{b} | 7^{c} | 8 |
| Cₘₐₓ (mg/mL) | 3.76 (0.395) | 14.6 (6.29) | 9.86 (3.68) |
| Cₘₐₓ/D | 0.063 (0.007) | 0.073 (0.032) | 0.049 (0.018) |
| (mg/mL/mg/kg) | | | |
| Tₘₐₓ (hr) | N/A | N/A | 24.5 (17.4) |
| AUC | 477 (62.7) | 1770 (741) | 1290 (434) |
| (mg · hr/mL) | | | |
| AUC/D | 7.95 (1.05) | 8.83 (3.7) | 6.46 (2.17) |
| (mg · hr/mL/mg/kg) | | | |

N/A = not applicable;
Cmax/D = dose-normalized maximum concentration;
AUC/D = dose-normalized area under the concentration-time curve;
Tmax = time to maximum concentration Data from Study TC13-084.
Tmax is not reported for IV dosing.
^{a}Two animals (at Day 22: 2002, 2502) excluded because of confirmed ADA response.
^{b}One animal (at Day 50: 2002) excluded because of confirmed ADA response; Animal 2502 was euthanized on Day 36.
^{c}One animal (at Day 22 and Day 50: 3502) excluded because of confirmed ADA response.

Two monkeys in the low dose group (60 mg/kg) and 1 monkey each in the 200 mg/kg IV and SC dose groups exhibited anti-ABBV-257 antibodies which correlated to a drop in exposure to the test article for the IV dosed animals. For the SC dosed ADA positive animal, the effect on the serum concentration profile was less obvious. Test-item induced ADA formation was not observed in any of the other animals and exposure of the animals to the test article was generally maintained.

### Example 19

### Toxicology Analysis of ABBV-257

The safety profile of ABBV-257 DVD-Ig binding protein was evaluated in a GLP-compliant 8-week (8 doses) cynomolgus monkey toxicology study. In addition, a GLP-compliant tissue cross reactivity study was conducted using human tissues. IV and SC injection site tolerability was assessed during the 8-week toxicology study. The local tolerances of the vehicle/placebo formulations (without ABBV-257) were also qualified in a GLP-compliant rabbit local tolerability study. Cynomolgus monkey was the only species utilized for toxicology studies due to insufficient cross reactivity of ABBV-257 to both TNF-α and IL-17 from mouse, rat, and rabbit species.

No adverse test article-dependent toxicities related to on-target or off-target binding of test article were observed during the GLP-compliant repeat-dose toxicology study using dose levels of 60 and 200 mg/kg IV, and 200 mg/kg SC.

During the 8-week toxicology study, one 60 mg/kg animal died following Dose 6. A comprehensive evaluation of the cumulative data from clinical observations, toxicokinetics, and anti-drug antibody analyses, serum circulating immune complex data, complement activation data, histologic evaluation of tissues, and immunohistochemical evaluation of immune complex deposition in tissues indicate the death was the result of exacerbation of an immune complex-mediated hypersensitivity reaction. The mortality was not attributed to a pharmacologic or toxicologic effect of test article administration.

Based upon a lack of adverse test article-related findings, the No Adverse Effect Level (NOAEL) during the 8-week repeat-dose toxicology study was 200 mg/kg/week among animals with sustained exposures. A summary of pivotal toxicology studies conducted with ABBV-257 is presented in Table 28.

TABLE 28
List of Pivotal Toxicology Studies Conducted with ABBV-257

| Type of | Species and | Method of | Duration | Doses^{a} |
|---|---|---|---|---|
| Study | Strain | Administration | of Dosing | (mg/kg/day) |
| Repeated- | Cynomolgus | IV, SC | 8 weeks | 60 IV, 200IV, |
| Dose | monkey | | | 200 SC once |
| Toxicity | | | | per week |
| Tissue | Human | — | — | — |
| Cross- | | | | |
| Reactivity | | | | |

Note that both the repeated-dose toxicity study and the tissue cross-reactivity study were GLP compliant.
IV = intravenous;
SC = subcutaneous;
IA = intra-arterial;
PV = paravenous;
IM = intramuscular
^{a}The NOAEL is underlined for GLP-compliant repeat-dose toxicity studies.

### Single Dose Toxicity

No single-dose toxicity studies were conducted. Analysis showed that no post-dose reactions or other ABBV-257-related effects were observed following the first dose of ABBV-257 among the 8-week repeat-dose toxicity studies described in the following section.

### Repeated Dose Toxicity: 8-Week Toxicology Study of ABBV-257 by Intravenous Bolus and Subcutaneous Injection in Cynomolgous Monkeys

An 8-week GLP-compliant toxicity study was conducted in male and female cynomolgus monkeys at dose levels of 0 (placebo/vehicle; IV and SC), 60 mg/kg, or 200 mg/kg once/week IV bolus injection (3 to 5 minutes) and 200 mg/kg once per week SC injection (8 total doses/regimen). A preceding 4-week (4 dose, once/week) non-GLP repeat dose toxicokinetic/tolerability study at a single dose level of 100 mg/kg once/week indicated that serum test article exposures could be maintained at this dose level for 4 weeks.

Study parameters during the 8-week GLP-compliant repeat dose toxicology study included clinical signs, injection site observations, body weights, food evaluation, ophthalmologic and electrocardiologic examinations, clinical pathology (hematology, coagulation, clinical chemistry, urinalysis), toxicokinetic and ADA analyses, ADA parameters, ADA isotyping, circulating serum immune complex (CIC) values, gross necropsy, organ weight, histopathology and immunohistochemistry evaluation of immune complex deposition in tissues.

No adverse test article-dependent toxicities related to on-target or off-target binding of test article were observed during the GLP-compliant repeat-dose toxicology study using dose levels of 60 and 200 mg/kg IV, and 200 mg/kg SC.

Serum test article concentrations and toxicokinetic parameters for ABBV-257 did not exhibit any gender specific differences. Toxicokinetic values increased in a dose level and dose route related fashion throughout the dosing period. The 200 mg/kg IV dose and route produced the highest exposures; correlating to a Day 50 C max of 14.6 mg/mL and an AUC₀₋₁₆₆ of 1770 mg·hr/mL.

Two 60 mg/kg IV animals (inclusive of 1 early death described below) and one 200 mg/kg IV animal exhibited ADA which corresponded to concurrently decreased ABBV-257 serum concentrations. Due to the ADA-altered systemic exposures, these three animals were excluded from mean toxicokinetic parameter calculations. One 200 mg/kg SC animal exhibited ADA that did not appear to negatively impact systemic exposure for this animal; therefore, the exposure data from this animal were not excluded from mean toxicokinetic calculations. The ADA observed in these 4 animals was IgG (not IgA, M, or E) isotype, and formed circulating immune complexes (ABBV-257/ADA complexes) in serum.

An acute post-dose response was present in a single female at 60 mg/kg/week IV, which lead to early death on Day 36 (approximately 15 minutes following Dose 6). The early death of this animal is most consistent with an immune complex-mediated hypersensitivity reaction based upon multiple study endpoints. Clinical signs following Dose 6 included unresponsiveness, no corneal reflex, faint heartbeat, and agonal breathing. The animal had IgG ADA titers corresponding to markedly decreased ABBV-257 concentrations, formation of circulating ABBV-257/ADA immune complexes, and complement activation following test article administration. Postmortem histopathologic changes suggestive of immune hypersensitivity included the following in the lung: minimal neutrophilic margination and thrombi in alveolar vessels, fibrin in alveoli, and mild histiocytic infiltration. Assessment of tissue-resident immune complex deposition by immunohistochemical techniques revealed that increased human IgG (ABBV-257), monkey IgG and/or IgM (interpreted as ADA)-containing granular deposits in phagocytic cells in 1 or more tissues which were consistent with an immune complex (ABBV-257/ADA) basis for the post-dosing reaction and associated pathology in this animal. The mortality was not attributed to a pharmacologic or toxicologic effect of test article administration.

In conclusion, excluding the 1 early death attributable to an immune-mediated hypersensitivity response, no ABBV-257 dependent adverse effects were observed during the study. The 200 mg/kg IV animals produced the highest exposures (AUC 1770 mg·hr/mL on Day 50), which is the NOAEL among animals with sustained exposures.

### Tissue Cross-Reactivity

GLP-compliant tissue cross-reactivity studies were conducted using fluorescein labeled ABBV-257 (2 and 10 μg/mL) and cryo-preserved tissues from human. At least 3 donor samples were evaluated for each tissue type. The tissue panel included all of the tissues identified in relevant regulatory guidance.

There was no fluorescein labeled ABBV-257 staining of the test human tissue cryosections, consistent with the low-grade expression of its target human epitopes in normal human tissues. There was no unexpected cross-reactivity. All assay control samples performed appropriately.

### Local Irritation

Test article injection site tolerance was evaluated during the 8-week repeat-dose toxicology studies. No injection site intolerance was observed via the IV and SC routes. A dedicated rabbit local tolerance study using ABBV-257 drug substance/drug product was not conducted.

### Example 20

### Study M14-355—a Phase 1 First-in-Human (FIH) Single Ascending Dose Study in Healthy Human Subjects

Clinical trial study M14-355 was performed and involved a single ascending dose, double-blind, randomized study planned for up to 40 healthy adult subjects to assess the safety, tolerability, and PK of ABBV-257 DVD-Ig binding protein with a single dose IV infusion or a single dose SC injection. Secondary objectives are to measure the ADA levels following a single IV or SC dose. An exploratory objective is to determine any change in biomarker assessments at multiple time points following study drug administration. The doses administered were 0.3 mg/kg (Group 1), 1.0 mg/kg (Group 2), and 3.0 mg/kg (Group 3) given IV and 0.3 mg/kg (Group 4) and 3 mg/kg (Group 4a) given SC.

Eighteen subjects received IV doses and 12 subjects received SC doses of ABBV-257. Ten subjects received placebo control (6 in the IV administration arm and 4 in the SC administration arm).

### Pharmacokinetics of ABBV-257 in Study M14-355

The mean and single-dose serum concentration-time profiles following an IV or SC dose of ABBV-257 are presented on a log-linear scale in FIG. 13 panel A and FIG. 13 panel B; see also Table 29.

TABLE 29
Mean (% CV) Pharmacokinetic Parameters Following a Single Dose of ABBV-257

| | Intravenous | | | | | | Subcutaneous | | | |
|---|---|---|---|---|---|---|---|---|---|---|
| | Group 1 | | Group 2 | | Group 3 | | Group 4 | | Group 5 | |
| Parameter | 0.3 mg/kg | | 1.0 mg/kg | | 3.0 mg/kg | | 0.3 mg/kg | | 3.0 mg/kg | |
| (Units) | N = 6 | | N = 6 | | N = 6 | | N = 6 | | N = 6 | |
| Cₘₐₓ (μg/mL) | 8.1 | (18) | 26.8 | (20) | 76.2 | (8) | 3.2 | (18) | 35.3 | (10) |
| Tₘₐₓ (hr)^{a} | 4.0 | (2-6) | 4.0 | (2-10) | 4.0 | (2-8) | 156 | (48-240) | 204 | (120-240) |
| Tmax (Day) | | | | | | | 6.5 | (2-10) | 8.5 | (5-10) |
| AUC_{0-Last} | 105 | (44) | 395 | (39) | 1460 | (35) | 90 | (53) | 982 | (38)^{b} |
| (μg · day/mL) | | | | | | | | | | |
| AUC_{0-inf} | 108 | (41) | 423 | (40)^{c} | 1545 | (43) | 91 | (53) | 1056 | (44)^{b} |
| (μg · day/mL) | | | | | | | | | | |
| CL (L/day)^{d} | 0.26 | (42) | 0.22 | (42)^{c} | 0.18 | (38) | 0.32 | (44) | 0.27 | (54)^{b} |
| t_{1/2} (Day)^{e} | 5.6 | (162) | 5.8 | (134) | 11.2 | (48) | 5.8 | (55) | 5.7 | (148)^{b} |
| Cₘₐₓ/Dose | 27.1 | (18) | 26.8 | (20) | 25.4 | (8) | 10.7 | (18) | 11.8 | (10) |
| (μg/mL)/(mg/kg) | | | | | | | | | | |
| AUCinf/Dose | 361 | (41) | 423 | (40)^{c} | 515 | (43) | 305 | (53) | 352 | (44)^{b} |
| (μg · day/mL)/(mg/kg) | | | | | | | | | | |

^{a}Median (range).
^{b}N = 4.
^{c}N = 5.
^{d}For SC dosing: apparent CL (CL/F: apparent total body clearance) is reported.
^{e}Harmonic mean (pseudo % CV); Terminal t½ may not be relevant because of the fast change in the slope at late time points.

The pharmacokinetics (Cmax and AUCinf) of ABBV-257 are slightly more than dose proportional following 0.3 to 3 mg/kg single dose range. The estimated bioavailability after SC administration was 74%.

The presence of ADA was measured with a validated immunoassay. Sampling for ADA occurred prior to ABBV-257 dosing (pre-dose) and following the single dose of ABBV-257 on Days 15, 22, 29, 36, 43, 57, 71 and 85. Complete preliminary ADA data are available for the first 4 dose groups and partial ADA data are available for the last dose group. ADA titers were detected in 23 out of 24 subjects in Groups 1 through 4. Of the 18 subjects who received ABBV-257 DVD-Ig binding protein in Groups 1 through 3, 9 had ADA associated with shorter half-life than the rest of the subjects, suggesting a negative impact of ADA on ABBV-257 exposure in these subjects. In the SC dose cohorts, 5 out of the 12 subjects who received ABBV-257 (4 subjects in Group 4 and 1 in Group 4a) had ADA associated with shorter half-life compared to the rest of the subjects with lower ADA titer values in these dose groups. ADA detected in the study did not impact the safety or tolerability profile of ABBV-257.

### Safety

Eighteen subjects received IV doses and 12 subjects received SC doses of ABBV-257 DVD-Ig binding protein. Seven of the 18 subjects (7/18, 38.9%) who received ABBV-257 IV reported one or more AEs compared to the 4 of 6 subjects (4/6, 66.7%) who received placebo IV. Of the 12 subjects who received ABBV-257 SC, 3 (25.0%) reported at least one AE as compared to none of the 4 placebo recipients.

Viral upper respiratory tract infection was the only preferred term reported for more than one subject (two subjects in the IV arm of the study, one placebo recipient and one subject who received a 0.3 mg/kg dose of ABBV-257). There were no deaths, SAEs, or AEs leading to discontinuation during the study. The only events considered possibly related to the study drug were injection site reaction in one subject in the ABBV-257 0.3 mg/kg SC group and hyperhidrosis in one subject in the ABBV-257 3.0 mg/kg IV group. Most AEs were mild in intensity; no severe AEs were reported. All AEs in the IV-dosed subjects were described as mild intensity and all were categorized as toxicity grade 1. One case of moderate post-traumatic pain and one case of mild rhabdomyolysis were reported in the 3.0 mg/kg SC group. This subject reported discomfort in muscles following weight lifting and alcohol consumption ten days following administration of study drug. Prior weight lifting and alcohol consumption of this subject were clinically asymptomatic and there were no concurrent laboratory abnormalities. Neither event was considered by the investigator as having a reasonable possibility of being study drug related.

A female subject in Group 3 IV dosing 3.0 mg/kg, experienced an allergic reaction described as erythema and itching in face and right hand starting 82 days post study drug administration, which was mild in intensity and was treated with steroids. The subject reported definite exposure to a pet that had been in contact with poison ivy the day before the onset of symptoms. The subject recovered after twelve days, and the allergic reaction was assessed as not related to study drug by the investigator.

Overall there was no apparent dose relationship in frequency, type, or intensity of AEs in Study M14-355 following ABBV-257 administration via IV or SC routes. All the infections reported in the study were mild in severity and not related to study drug. There were no systemic hypersensitivity reactions reported. Antidrug antibodies detected in the study did not appear to impact the AE profile of ABBV-257.

All treatment-emergent adverse events experienced by at least 1 subject receiving ABBV-257, regardless of causality, include gastrointestinal disorders (abdominal pain, diarrhoea, vomiting), general disorders and administration site conditions (fatigue, infusion site, haematoma, injection site reaction, local swelling), immune system disorders (hypersensitivity), infections and infestations (viral upper respiratory tract infection), injury, poisoning and procedural complications (eye, penetration, ligament sprain, post-traumatic pain, tooth fracture), musculoskeletal and connective tissue disorders (rhabdomyolysis), nervous system disorders (presyncope), respiratory, thoracic and mediastinal disorders (epistaxis, oropharyngeal pain), and skin and subcutaneous tissue disorders (hyperhidrosis).

In general, the incidence of potentially clinically significant laboratory values was low. There were no AEs related to laboratory abnormalities. Grade 3 or 4 potentially clinically significant laboratory values were reported for neutrophils, creatine phosphokinase (CPK), alanine aminotransferase (ALT), aspartate aminotransferase (AST), triglycerides, and urine protein with the highest incidence of such values occurring for CPK. With regard to the potentially clinically significant laboratory values, there was no obvious pattern or evident correlation with dose or route of administration. There were no apparent differences in the potentially clinically significant laboratory values between placebo and ABBV-257 DVD-Ig binding protein.

Of the IV-dosed subjects, two subjects (one placebo recipient and one subject in the 0.3 mg/kg dose group) had a potentially clinically significant decrease in heart rate (<50 bpm and ≧15 bpm decrease) and one subject in the 1.0 mg/kg dose group had a potentially clinically significant increase in heart rate (>120 bpm and ≧15 bpm increase). None of these observations was considered clinically significant and no AEs reported in the study were related to vital signs or ECG findings. There were no other subjects with a potentially clinically significant vital sign or ECG value.

### Summary of First-in-Human Clinical Study M14-355

Examples herein discussed the FIH study of ABBV-257 (Study M14-355), which enrolled healthy adult volunteers to assess the safety, tolerability, pharmacokinetics, and ADA profile of a single dose of ABBV-257 without the confounding effects of concomitant disease or therapy. Potential risks with this study were addressed by the protocol-defined inclusion and exclusion criteria, study design features, and monitoring procedures outlined herein, and specified in the protocol. The last subject/last visit in the study was completed and data analysis and reporting were ongoing. Preliminary safety results are available.

Part 1 of Study M14-355 was a randomized, double-blind, placebo-controlled design to assess the safety, tolerability, pharmacokinetics and immunogenicity (via ADA assessment) of a single IV infusion of ABBV-257. This part of the study was conducted in 24 subjects in 3 groups (Groups 1 to 3), with 8 subjects in each group. Within each group, 6 subjects were randomized to receive ABBV-257 and 2 subjects received matching placebo. The ABBV-257 dose administered in Group 1 was 0.3 mg/kg IV. The subsequent ABBV-257 doses were 1.0 mg/kg and 3.0 mg/kg IV for Groups 2 and 3, respectively.

Part 2 of Study M14-355 was a randomized, double-blind, placebo-controlled design to assess the safety, tolerability, pharmacokinetics and immunogenicity (via ADA assessment) of a single SC injection of ABBV-257. This part of the study was conducted in 16 subjects in two groups (Groups 4 and 4a), with eight subjects in each group. Within each group, six subjects were randomized to receive ABBV-257 and 2 subjects received matching placebo. The ABBV-257 doses administered are 0.3 mg/kg SC and 3 mg/kg SC for Groups 4 and 4a, respectively.

Preliminary pharmacokinetic data indicated ABBV-257 exposure to be slightly more than dose proportional following 0.3 to 3.0 mg/kg dose range. The preliminary bioavailability estimate after SC administration was ˜74%. The majority of subjects in the FIH study had detectable ADA, within 2 weeks of dosing. ADA detected in the study did not impact the safety or tolerability profile of ABBV-257.

Preliminary safety data indicate that ABBV-257 has an acceptable safety and tolerability profile. There were no deaths, SAEs, or discontinuations due to AEs during Study M14-355. Most of the AEs reported have been mild in severity. All infections were mild in severity and not related to study treatment. No systemic hypersensitivity reactions were reported. The only AE experienced by more than one subject was viral upper respiratory tract infection, which occurred in two subjects in the IV group (one placebo recipient and one subject who received a 0.3 mg/kg dose of ABBV-257). In general, the incidence of potentially clinically significant abnormal laboratory values was low, with CPK being the most common potentially clinically significant abnormal laboratory value. Laboratory abnormalities seen following placebo or ABBV-257 were comparable. No concerning patterns of AEs or laboratory findings were reported. Overall there was no apparent dose relationship in frequency, type, or intensity of AE s or laboratory abnormalities in Study M14-355 following IV or SC administration of ABBV-257. There were no observed clinically significant vital sign or ECG abnormalities. ADA detected in the study did not impact the safety or tolerability profile of ABBV-257. No dose limiting toxicities were observed during the study.

### Safety

The potential safety concerns for administration of ABBV-257 are the risk of systemic hypersensitivity reactions and an increased risk of infection. There was no evidence for either of these safety concerns in Study M14-355. Additionally, with over 100 subjects currently exposed to single or multiple doses of another DVD-Ig targeting TNF and IL-17, no systemic hypersensitivity reactions have been reported. One subject in the 3.0 mg/kg IV group experienced a mild localized rash, redness, itching on the right side of her face and right hand with onset 82 days after received ABBV-257 3.0 mg/kg IV. The subject recovered after 12 days, and the event reported as an allergic reaction was assessed as not related to study drug by the investigator. Infections reported during Study M14-355 were mild viral upper respiratory tract infections assessed as not related to study treatment, which occurred in two subjects (one placebo recipient and one subject in the 0.3 mg/kg dose group). However, several precautions are taken in the planned multiple ascending dose Study M14-439 to mitigate the risk of potential systemic hypersensitivity reactions with ABBV-257. To address the risk for infection or hypersensitivity reactions in humans who receive ABBV-257, the study protocol implements enrollment criteria, screening procedures, and a clinical schedule and monitoring plan to mitigate, monitor, and manage potential hypersensitivity reactions, other systemic reactions, and infections.

The risk of other AEs that have been associated with the anti-TNF agents, including malignancy, central nervous system demyelinating disease, pancytopenia (including aplastic anemia), worsening or new onset heart failure, and lupus-like syndrome, was low given the limited duration of exposure in this study in healthy volunteers, the application of protocol-specified exclusion criteria and safety monitoring procedures in the study protocol. No such events were reported in Study M14-355.

### Example 22

### Clinical Study Protocol M14-439—a Randomized, Double-Blind, Placebo-Controlled Study in Subjects with Rheumatoid Arthritis to Evaluate the Safety, Tolerability and Pharmacokinetics of Multiple Doses of ABBV-257

This Phase 1, randomized, double-blind, placebo-controlled, multiple-dose study is designed to assess the safety, tolerability, pharmacokinetics and immunogenicity of different dose levels of ABBV-257 given with methotrexate (MTX). Adult male and female subjects with RA are selected to participate in the study according to the selection criteria.

The study was designed to enroll 24 subjects to meet scientific and regulatory objectives without enrolling an undue number of subjects in alignment with ethical considerations. After meeting the selection criteria, enrolled subjects are randomly assigned in 3:1 ratio to either ABBV-257 or Placebo, in sequential dose groups as shown in Table 30.

TABLE 30
Planned Dose Groups

| | | Number of Subjects |
|---|---|---|
| Group^{a} | Regimen^{b,c} | Active:Placebo |
| 1 | 30 mg of ABBV-257 or placebo | 6:2 |
| | SC EOW dosing (4 doses) | |
| 2 | 100 mg of ABBV-257 or placebo | 6:2 |
| | SC EOW dosing (4 doses) | |
| 3 | 300 mg of ABBV-257 or placebo | 6:2 |
| | SC EOW dosing (4 doses) | |

^{a}Subjects may not participate in more than one dosing group
^{b}Dose level or dosing frequency may be adjusted based on the available safety, tolerability, and PK data from previous dose group(s).
^{c}Subjects receive their stable MTX dose weekly.

Study drug (ABBV-257 or placebo) is administered on Study Days 1, 15, 29, and 43 for the EOW dosing. The first three subjects of the first dose group are dosed at least 24 hours apart. The remaining subjects within a dose group may be dosed up to 2 subjects per day. Subjects continue their weekly MTX dosing throughout participation in the study. Dosing for Groups 2 and 3 is sequentially enabled upon the review of safety data through administration of the study drug at approximately Day 15 of the last subject in the precedent dose group. The subsequent dosing scheme may be adjusted (e.g., dosing interval, number of doses) based on PK and safety data from previous group(s). Subjects are confined to the study site and supervised for periods of approximately 72 hours for the first and last doses of study drug. Confinement for the first dose begins on Study Day −1. Subjects remain at the study site and are supervised for at least 2 hours following the second and third doses of study drug. Confinement for the last dose begins on Day 42. Each confinement period ends after completion of all study procedures on the scheduled day of discharge.

Subjects have outpatient visits between confinement periods as indicated in Table 39. Safety is assessed throughout the study. Pharmacodynamic effects of ABBV-257 are investigated by exploratory disease response measures, biomarkers and fluorescence optical imaging (FOI) in patients with tender/swollen joints in hands as indicated in Table 39. From subjects who consent, a blood sample is collected to obtain a sample of genetic material (DNA/RNA). These DNA/RNA samples may be used to study genetic factors contributing to the subject's response to ABBV-257 in terms of pharmacokinetics, pharmacodynamics, and safety.

### Selection of Study Population

Subjects undergo screening procedures within 30 days prior to initial study drug administration. Adult male and female subjects in general good health who meet the inclusion criteria and who do not meet any of the exclusion criteria are eligible for enrollment into the study. Subjects that initially screen-fail for the study are permitted to re-screen one time following a repeat of all screening procedures with the possible exceptions noted below. The subject must meet all inclusion and none of the exclusion criteria at the time of re-screening in order to qualify for the study. There is no minimum period of time a subject must wait to re-screen for the study. If the subject had a complete initial screening visit including the assessment of a PPD test (or equivalent) and chest x-ray (CXR), these two tests are not required to be repeated for the re-screening visit. Adult male and female subjects with RA are selected to participate in the study according to the selection criteria.

### Inclusion Criteria

A subject is eligible for study participation if he/she meets the following criteria:

1. Male or female and age is between 18 and 75 years, inclusive.
2. Diagnosis of RA based on either the 1987 revised ACR classification criteria or the 2010 American College of Rheumatology (ACR)/European League against Rheumatism (EULAR) criteria ≧3 months.
3. Except for MTX, the subject must have discontinued all disease modifying anti-rheumatic drugs (DMARD) for at least 5 half-lives before the first dose of study drug, and undergone cholestyramine washout if received Leflunomide within the past 3 months.
4. Subject must have been on methotrexate therapy >3 months and on a stable dose (7.5-25 mg/week) for at least 4 weeks prior to the first dose of study drug. Subject must be able to continue on stable dose of MTX for the duration of study participation.
5. If female, subject must meet one of the following criteria: Postmenopausal (defined as no menses for at least 1 year, with no alternate cause for amenorrhea); Surgically sterile (bilateral oophorectomy or hysterectomy). Women not in one of the above two categories are considered of child bearing potential and must use an approved birth control.
6. Females must have negative results for pregnancy tests performed: at Screening on a urine specimen obtained within 30 days prior to initial study drug administration, and prior to dosing on a serum sample obtained on Study Day −1.
7. If male, subject must agree not to donate sperm starting on the first day of confinement until 5 months after last dose of study drug.
8. If male, subject (including those who have had vasectomies) should use condoms from the first dose of study drug until 5 months after the last dose of study drug.
9. Body Mass Index (BMI) is 19 to 35, inclusive. (BMI is calculated as weight [kg] divided by height [m²].)
10. Judged to be in good general health as determined by the Investigator based upon the results of medical history, laboratory profile, physical examination and 12-lead electrocardiogram (ECG) performed at Screening.
11. Must voluntarily sign and date each informed consent, approved by an Independent Ethics Committee (IEC)/Institutional Review Board (IRB), prior to the initiation of any screening or study-specific procedures.

### Exclusion Criteria

A subject is eligible for study participation if he/she meets any of the following criteria:

1. Female who is pregnant or breastfeeding.
2. Female subject who is considering becoming pregnant during the study or for approximately 5 months after the last dose of study drug or male subject who is considering fathering a child during the study or for approximately 5 months after the last dose of study drug.
3. History of clinically significant drug or alcohol abuse in the 6 months prior to initial study drug administration.
4. Positive screen for drugs of abuse or alcohol at Screening or upon initial confinement.
5. Evidence of anti-ABBV-257 antibody results in a pre-study serum sample.
6. History of significant allergic reaction or significant sensitivity to any constituents of the study drug formulation; or history of anaphylactic reaction to any agent (e.g., food products and bee sting); or history of a major reaction to any IgG-containing product; or known or suspected allergy to FOI fluorescent agent or iodine.
7. Evidence of dysplasia or history of malignancy (including lymphoma and leukemia) other than a successfully treated non-metastatic cutaneous squamous cell or basal cell carcinoma or localized carcinoma in situ of the cervix.
8. History of persistent chronic or active infection(s) requiring hospitalization or treatment with intravenous or oral antimicrobials/antibiotics within 30 days prior to initial study drug administration.
9. HBs Ag positive (++) or detected sensitivity on the HBV-DNA PCR qualitative test for HBc Ab/HBs Ab positive subjects; or history or evidence of active hepatitis C infection.
10. History of or positive screening test for human immunodeficiency virus (HIV Ab) infection; or a history of any genetic, congenital, or acquired immunodeficiency syndrome. Negative HIV status is confirmed at Screening and the results are maintained confidentially by the study administration.
11. History or evidence of active tuberculosis (TB). Subjects are evaluated for latent TB infection. Subject must demonstrate absence of TB infection or exposure by a negative QuantiFERON-TB Gold at Screening.
12. In the opinion of the investigator, the subject has evidence of risk factors for latent TB.
13. Has received any investigational drug product of chemical or biologic nature within 30 days or 5 half-lives of the drug (whichever is longer) prior to initial study drug administration.
14. Has a history of any clinically significant respiratory, renal, hepatic, gastrointestinal, hematologic disorder, non-healing wounds or recurrent poor wound healing, or any uncontrolled medical illness, neurologic symptoms of demyelinating disease.
15. Recent history of a psychiatric illness that in the opinion of the Investigator could interfere with compliance to the protocol.
16. Known medical diagnosis of persistent asthma, chronic obstructive pulmonary disease if it could impact participation in the study; or significant atopy requiring daily therapy; history or diagnosis of mastocytosis or clonal mast cell disorder.
17. Febrile illness within 1 week prior to dosing.
18. History of chronic recurrent or persistent infections (including mucocutaneous candidiasis).
19. Has undergone major surgery within the 2 months prior to the initial study drug administration.
20. Donation or loss of 550 mL or more blood volume (including by plasmapheresis) or receipt of a transfusion of any blood product within 8 weeks prior to initial study drug administration.
21. Clinically significant abnormal ECG including ECG with QTcF>450 msec, PR interval >220 msec, or other clinically significant baseline abnormalities as judged by the Investigator at Screening or Study Day −1.
22. Myocardial infarction, coronary stenting, or CVA within the 1 year prior to initial study drug administration or greater than Class 1 angina pectoris or clinically significant aortic stenosis.
23. Cardiac failure at time of Screening >NYHA Class 2.
24. Confirmed systolic blood pressure measurement >160 mmHg systolic and >100 mmHg diastolic on Study Day −1.
25. History of diabetes mellitus (DM), HbA1c of ≧6.5% at Screening or fasting plasma glucose (FPG)≧126 mg/dL (7.0 mmol/L) at Screening.
26. Confirmed hemoglobin≦9 gm/dL or platelet count <100,000 μ/L or WBC <3000 μ/L or absolute neutrophil count <1500 μ/L at Screening.
27. Clinically significant abnormal screening laboratory results as evaluated by the Investigator, including serum values of AST or ALT greater than 2.25× the upper limit of normal, or creatinine greater than 1.5× the upper limit of normal, or absolute neutrophil count <1500 μ/L.
28. Subject has received vaccination with a live viral agent (including live attenuated influenza vaccine via nasal spray)≦to 1 month prior to Screening or require vaccination during study participation and up to approximately 5 months (at least 5× the estimated half-life for ABBV-257) after the last dose of study drug.
29. Subject is unable to washout prohibited medications.
30. Concurrent use of other immunosuppressant medications other than those allowed as specified in the protocol.
31. Subject has any medical condition or illness other than RA that is not well controlled with treatment that would, in the opinion of the Investigator, preclude study participation or interfere with other symptoms of RA.
32. Subject who has been legally institutionalized.
33. Current enrollment in another investigational study.
34. Consideration by the Investigator, for any reason that the subject is an unsuitable candidate to receive ABBV-257.

### Prior and Concomitant Therapy

Subject must have been on methotrexate therapy >3 months and on a stable dose (7.5-25 mg/week) for at least 4 weeks prior to the first dose of study drug. Subjects continue taking MTX as prescribed in addition to receiving study drug (ABBV-257 or placebo) throughout the duration of the study. Reduction in the dose of MTX is not allowed. If the subject cannot tolerate their dose of MTX, he/she is discontinued from the study. If a subject reports taking any over-the-counter or prescription medications, vitamins and/or herbal supplements or if administration of any medication becomes necessary from 2 weeks prior to study drug administration through the end of the study, the name of the medication, dosage information including dose, route and frequency, date(s) of administration including start and end dates, and reason for use is recorded, and the study designated physician is notified.

Study drug is administered as follows shown in Table 31:

TABLE 31
Dosing Schedule

| Group | Dose | Dosing Days |
|---|---|---|
| 1 | 30 mg SC | 1, 15, 29, 43 |
| 2 | 100 mg SC | 1, 15, 29, 43 |
| 3 | 300 mg SC | 1, 15, 29, 43 |

Dosing for Groups 2 and 3 is enabled after all subjects in the previous group have satisfactorily completed at least a minimum of 1 week of safety assessments after the last subject's second dose is administered. The escalation scheme may be adjusted (e.g., dosing interval, number of doses) based on PK and safety from preceding dose groups based on data from previous group(s). The number of injections per subject varies by dose level. Depending on the number of syringes required for each subject, one injection per site is administered subcutaneously in the following order (as needed):

1. Left upper quadrant of the abdomen
2. Right upper quadrant of the abdomen
3. Left anterior proximal thigh
4. Right anterior proximal thigh
5. Left lower quadrant of the abdomen
6. Right lower quadrant of the abdomen

The areas to avoid for SC injections include: any blood vessels, thickening or tenderness of skin, scars, fibrous tissue, lesions, stretch marks, bruises, redness, nevi, or other skin imperfections. Injection sites should be at least 1 inch apart and at least 2 inches from the navel. The subject should remain in a supine position for at least 30 minutes following study drug administration. The time of each drug administration is recorded to the nearest minute.

### Identity of Investigational Products

Information about the ABBV-257 formulations to be used in this study is presented in Table 32.

TABLE 32
Identity of Investigational Products

| Investiga- | ABBV-257 50 mg | Placebo for ABBV-257 |
|---|---|---|
| tional | Powder for | 50 mg Powder for |
| Product | Injection Vial | Injection Vial |
| Dosage form | Powder for solution for | Powder for solution for |
| | injections in vials | injection in vials |
| Strength | 50 mg/mL when | N/A |
| (mg) | reconstituted with 1.2 | |
| | mL of sterile water | |
| | for injection | |
| Mode of | Subcutaneous injection | Subcutaneous injection |
| Administration | | |

ABBV-257 50 mg powder for solution for injection vial and matching placebo for ABBV-257 50 mg powder for solution for injection vial is reconstituted with sterile water for injection described herein.

### Preparation/Reconstitution of Dosage Forms

The ABBV-257 drug product (active and placebo) is provided as a powder in vials. Each vial of ABBV-257 and placebo is reconstituted with 1.2 mL of sterile water for injection to provide a 50 mg/mL ABBV-257 active or a placebo solution. The ABBV-257 drug product is dosed as a fixed dose. The total volume administered is dependent upon the assigned dose. The ABBV-257 drug product and placebo solutions are administered via subcutaneous (SC) injection. Specific dose preparation and documentation details are provided to the site pharmacy outside of this protocol.

### Method of Assigning Subjects to Treatment Groups

The randomization schedule is computer-generated before the start of the study by the Statistics Department. As they are randomized in the study, subjects of each group are assigned unique, consecutive numbers beginning with 1001 for Group 1, 2001 for Group 2 and 3001 for Group 3. Within each group, subjects are randomized in a 3:1 ratio to receive either ABBV-257 or matching placebo. If additional groups are added (beginning with Group 4), subjects of each group are assigned unique, consecutive numbers (beginning with 4001) for randomization to ABBV-257 or matching placebo.

### Selection and Timing of Dose for Each Subject

Selection of the dose for this study is discussed herein. Within a group, the subjects assigned to ABBV-257 are administered the same dose. ABBV-257 or matching placebo is administered on Study Days 1, 15, 29, and 43 prior to MTX dose.

### Blinding

The study is conducted in a double-blind manner such that the principal investigator, study coordinator, subjects and the study team are blinded to the treatment assignments. Placebo in its powder form is identical in appearance to the ABBV-257 powder form; however, both are delivered to the study drug preparation designee or pharmacist in an open-label format for further preparation.

The study designated statisticians assigned to this study are unblinded to allow for expedited review of the safety and pharmacokinetic data.

### Appropriateness of Measurements

Standard pharmacokinetic, statistical, clinical, and laboratory procedures are utilized in this study. Disease response, biomarker and imaging data are collected for exploratory analysis.

### Suitability of Subject Population

This study enrolls male and female subjects who have been diagnosed with RA and have been on MTX for at least 3 months and are on a stable regimen of MTX (7.5-25 mg/week) for at least 4 weeks. The study population selected in this study reflects the standard population for RA trials with new intervention.

### Selection of Doses in the Study

To date, ABBV-257 has been evaluated in the first-in-human (FIH) single ascending dose study (Study M14-355) in healthy subjects that included IV doses of 0.3, 1.0 and 3.0 mg/kg and SC doses of 0.3 and 3 mg/kg. This study recently completed dosing; preliminary analysis of safety data demonstrate a favorable safety profile up to 3 mg/kg following IV or SC administration. There were no severe or serious adverse events following study treatment. No subjects prematurely discontinued due to adverse events to ABBV-257. All infections were mild in severity and no systemic hypersensitivity reactions were reported. Study M14-355 safety data are reviewed more in detail at ABBV-257 investigator's brochure.

When ABBV-257 was tested in a GLP 8-week monkey toxicology study, there were no first-dose infusion reactions at any of the dose levels tested (60, and 200 mg/kg IV and 200 mg/kg SC), nor any reactions observed with the SC route of administration. The NOAEL dose was 200 mg/kg IV and resulted in a Cₘₐₓ and estimated AUC_{0-14day} of 14,600 μg/mL and 147,500 μg·day/mL, respectively. The estimated AUC at the NOAEL provide 1222- and 122-fold safety margin relative to the steady-state AUC at the starting dose of 30 mg/kg EOW and highest dose of 300 mg/kg EOW, respectively. In addition, the preliminary estimate of AUC_{inf} at the highest IV dose in the SAD study provide 1.3-fold margin from the predicted steady-state exposure at the highest dose proposed for this study.

ABBV-257 is a high-affinity bispecific recombinant human molecule with TNF-binding properties comparable to those of the monoclonal anti-TNF antibody adalimumab. Affinities for TNF are 5 pM with ABBV-257, 8 pM with a distinct TNF/IL-17 DVD-Ig and 30 pM with adalimumab. In a Phase 1 clinical trial of patients with RA administered a single IV dose of adalimumab, a clinical response was observed at the lowest dose tested, 0.5 mg/kg, with greater response observed at all higher doses tested, up to 10 mg/kg. In a Phase 3 study of multiple, SC doses of adalimumab monotherapy for RA, a successively greater clinical response was observed with increasing exposure in doses corresponding to a dose range of approximately 0.3 to 1.14 mg/kg. This range encompasses the indicated starting dose for adalimumab in RA, 40 mg every other week (EOW), which corresponds to approximately 0.6 mg/kg.

### Adverse Events

The investigator monitors each subject for clinical and laboratory evidence of adverse events on a routine basis throughout the study. The investigator assesses and record any adverse event in detail including the date of onset, event diagnosis (if known) or sign/symptom, severity, time course (end date, ongoing, intermittent), relationship of the adverse event to study drug, and any action(s) taken. For serious adverse events considered as having “no reasonable possibility” of being associated with study drug, the investigator provides another cause of the event. For adverse events to be considered intermittent, the events must be of similar nature and severity. Adverse events, whether in response to a query, observed by site personnel, or reported spontaneously by the subject are recorded. All adverse events are followed to a resolution.

### INCORPORATION BY REFERENCE

The present invention incorporates by reference in their entirety techniques well known in the field of molecular biology, drug delivery, immunology, molecular biology and cell biology. These techniques include, but are not limited to, techniques described in the following publications: Ausubel et al. (eds.) (1993) Current Protocols in Molecular Biology, John Wiley & Sons, NY; Ausubel et al. (eds.) (1999) Short Protocols In Molecular Biology John Wiley & Sons, NY (ISBN 0-471-32938-X); Smolen and Ball (eds.) (1984) Controlled Drug Bioavailability Drug Product Design and Performance, Wiley, NY; Giege and Ducruix (1999) Crystallization of Nucleic Acids and Proteins, a Practical Approach, 2nd ed., pp. 20 1-16, Oxford University Press, NY; Goodson (1984) Medical Applications of Controlled Release, vol. 2, pp. 115-138; Hammerling et al. (1981)Monoclonal Antibodies and T-Cell Hybridomas 563-681 (Elsevier, N.Y.; Harlow et al. (1988) Antibodies: A Laboratory Manual, (Cold Spring Harbor Laboratory Press, 2nd ed.; Kabat et al. (1987) Sequences of Proteins of Immunological Interest (National Institutes of Health, Bethesda, Md.; Kabat et al. (1991) Sequences of Proteins of Immunological Interest, Fifth Edition, U.S. Department of Health and Human Services, NIH Publication No. 91-3242; Kontermann and Dubel (eds.) (2001) Antibody Engineering Springer-Verlag, NY 790 pp. (ISBN 3-540-41354-5); Kriegler (1990) Gene Transfer and Expression, A Laboratory Manual, Stockton Press, NY; Lu and Weiner (eds.) (2001) Cloning and Expression Vectors for Gene Function Analysis BioTechniques Press. Westborough, Mass. 298 pp. (ISBN 1-881299-21-X); Langer and Wise (eds.) (1974) Medical Applications of Controlled Release, CRC Pres., Boca Raton, Fla.; Old and Primrose (1985) Principles of Gene Manipulation: An Introduction To Genetic Engineering (3d Ed.) Blackwell Scientific Publications, Boston, Mass. Studies in Microbiology; V. 2:409 pp. (ISBN 0-632-01318-4); Sambrook et al. (eds.) (1989) Molecular Cloning: A Laboratory Manual (2d Ed.) Cold Spring Harbor Laboratory Press, NY, Vols. 1-3 (ISBN 0-87969-309-6); Robinson (ed.) (1978) Sustained and Controlled Release Drug Delivery Systems, Marcel Dekker, Inc., NY; Winnacker (1987) from Genes To Clones: Introduction To Gene Technology; VCH Publishers, NY (translated by Horst Ibelgaufts), 634 pp. (ISBN 0-89573-614-4).

Further, the contents of all cited references (including literature references, patents, patent applications, and websites) that maybe cited throughout this application are hereby expressly incorporated by reference in their entirety for any purpose, as are the references cited therein.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

1. A method for treating a subject having rheumatoid arthritis (RA), the method comprising the step of administering to the subject a binding protein that specifically binds both IL-17 and TNF-α.

2. The method of claim 1, wherein the binding protein is a dual variable domain immunoglobulin (DVD-Ig™) protein and/or wherein the subject is resistant to treatment with at least one disease-modifying antirheumatic drug (DMARD).

3. The method of claim 1, wherein the binding protein comprises the variable heavy (VH) complementarity determining regions (CDRs) for binding TNF-α from the amino acid sequence of SEQ ID NO: 5 and/or the VH CDRs for binding IL-17 from the amino acid sequence of SEQ ID NO: 7.

4. The method of claim 1, wherein the binding protein comprises the CDRs of the amino acid sequence of SEQ ID NO: 4 or the binding protein comprises the amino acid sequence of SEQ ID NO: 4.

5. The method of claim 1, wherein the binding protein comprises the VL CDRs for binding TNF-α from the amino acid sequence of SEQ ID NO: 10 and/or the VL CDRs for binding IL-17 from the amino acid sequence of SEQ ID NO: 12.

6. The method of claim 1, wherein the binding protein comprises the CDRs of the amino acid sequence of SEQ ID NO: 9 or the binding protein comprises the amino acid sequence of SEQ ID NO: 9.

7. The method of claim 1, wherein the binding protein further comprises a constant region.

8. The method of claim 1, wherein the method further comprises the step of administering to the subject a DMARD.

9. The method of claim 8, wherein the DMARD is selected from the group consisting of methotrexate, sulfasalazine, cyclosporine, leflunomide, hydroxychloroquine, and zathioprine.

10. The method of claim 1, wherein the binding protein is administered subcutaneously.

11. The method of claim 1, wherein the binding protein is administered intravenously.

12. The method of claim 1, wherein the binding protein is administered at a dosage selected from the group consisting of about: 0.1 milligram per kilogram of subject weight (mg/kg), 0.3 mg/kg, 1.0 mg/kg, 1.5 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg; and 24 mg/kg.

13. The method of claim 1, wherein the binding protein is administered at a dose of from about 0.5 mg/kg to about 10 mg/kg.

14. The method of claim 1, wherein the binding protein is administered at a dose selected from the group consisting of: about 0.5 mg/kg, about 1 mg/kg, about 1.5 mg/kg, and about 3 mg/kg.

15. The method of claim 1, wherein the binding protein is administered at a total dose selected from the group consisting of: about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, about 325-350 mg, about 350-375 mg, or about 375-40 mg of the binding protein.

16. The method of claim 1, wherein the binding protein is administered at least once over a period of time selected from the group consisting of: every: day, every other day, every week, every other week, every two weeks, every three weeks, every month, and every two months.

17. The method of claim 1, wherein administering the binding protein improves at least one a negative condition or symptom in the subject associated with rheumatoid arthritis.

18. The method of claim 17, wherein the negative condition or symptom is selected from the group consisting of: an autoimmune response, inflammation, stiffness, pain, bone erosion, osteoporosis, joint deformity, joint destruction, a nerve disorder, scarring, a cardiac disorder, a blood vessel disorder, high blood pressure, fatigue, anemia, weight loss, an abnormal temperature, a lung disorder, a kidney disorder, a liver disorder, an ocular disorder, a skin disorder, an intestinal disorder, and an infection.

19. The method of claim 1, wherein administrating the binding protein to the subject improves a score or criteria of at least one rheumatoid arthritis metric in the subject.

20. The method of claim 19, wherein the rheumatoid arthritis metric is selected from the group consisting of: Physician Global Assessment of Disease Activity; Patient Reported Outcome; a Health Assessment Questionnaire (HAQ-DI); a patient global assessment of disease activity (VAS); measurement or presence of an anti-drug antibody (ADA); tender joint count (TJC); swollen joint count (SJC); patient's assessment of pain; Work Instability Scale for Rheumatoid Arthritis; Short Form Health Survey (SF-36); American College of Rheumatology, ACR, (e.g., ACR20, ACR50, and ACR70); proportion of subjects achieving Low Disease Activity (LDA); Disease Activity Score 28; DAS28 based on C-reactive protein; Clinical Disease Activity Index (CDAI); simple disease activity index (SDAI); and Clinical Remission criteria.

21. A method for treating rheumatoid arthritis in a human subject, the method comprising the step of administering to the human subject a binding protein that specifically binds both TNF-α and IL-17, wherein the binding protein comprises a variable heavy chain comprising an amino acid sequence of SEQ ID NO: 4 and a variable light chain comprising an amino acid sequence of SEQ ID NO: 9, wherein the binding protein is administered in a dose to achieve
(a) an area under the curve (AUC) of between about 1 and about 500 μg·day/mL;
(b) a serum or plasma half-life (T_{1/2}) of at least about 2 to 20 days;
(c) a time point to maximum observed serum concentration (Tmax) of between about 1 days and about 10 days;
(d) a maximum observed serum concentration (Cmax) of between about 0.5 and about 400 μg/mL;
(e) an improvement of a negative condition or symptom associated with rheumatoid arthritis; and/or
(f) an improvement a score or criteria of one or more rheumatoid arthritis metric.

22. The method of claim 21, wherein the AUC is between about 17 and about 448 μg·day/mL.

23. The method of claim 21 or 22, wherein the T_{1/2} is at least about 5 and about 11 days.

24. The method of claim 21, wherein the Tmax is between about 1 and 7 days.

25. The method of claim 21, wherein the Cmax is between about 2 and about 81 μg/mL.

26. The method of claim 21, wherein the negative condition or symptom is selected from the group consisting of: an autoimmune response, inflammation, stiffness, pain, bone erosion, osteoporosis, joint deformity, joint destruction, scarring, a cardiac disorder, a blood vessel disorder, high blood pressure, fatigue, anemia, weight loss, an abnormal temperature, a nerve disorder, a lung disorder, a kidney disorder, a liver disorder, an ocular disorder, a skin disorder, an intestinal disorder, and an infection.

27. The method of claim 21, wherein the rheumatoid arthritis metric is selected from the group consisting of: Physician Global Assessment of Disease Activity; Patient Reported Outcome; a Health Assessment Questionnaire (HAQ-DI); a patient global assessment of disease activity (VAS); measurement or presence of an anti-drug antibody (ADA); tender joint count (TJC); swollen joint count (SJC); patient's assessment of pain; Work Instability Scale for Rheumatoid Arthritis; Short Form Health Survey (SF-36); American College of Rheumatology, ACR, (e.g., ACR20, ACR50, and ACR70); proportion of subjects achieving Low Disease Activity (LDA); Disease Activity Score 28; DAS28 based on C-reactive protein; Clinical Disease Activity Index (CDAI); simple disease activity index (SDAI); and Clinical Remission criteria.

28. The method of claim 21, wherein the subject is resistant to treatment with at least one disease-modifying antirheumatic drug (DMARD).

29. The method of claim 28, wherein the DMARD is selected from the group consisting of methotrexate, sulfasalazine, cyclosporine, leflunomide, hydroxychloroquine, and zathioprine.

30. The method of claim 21, wherein administering the binding protein comprises intravenously injecting the binding protein.

31. The method of claim 21, wherein administering the binding protein comprises subcutaneously injecting the binding protein.

32. The method of claim 21, wherein administering the binding protein is by at least one mode selected from the group consisting of: parenteral, subcutaneous, intramuscular, intravenous, intra-articular, intra-abdominal, intra-capsular, intra-cartilaginous, intra-osteal, intrapelvic, intraperitoneal, intrasynovial, intravesical, bolus, topical, oral, and transdermal.

33. The method of claim 21, wherein the binding protein is administered every day, every two days, twice per week, once per week, every two weeks, every other week, every three weeks, every month, every two months, or every few months.

34. The method of claim 21, wherein the method further comprises administering another therapeutic agent to the subject.

35. The method of claim 34, wherein the therapeutic agent comprises a DMARD.

36. The method of claim 21, wherein the binding protein is administered at a dosage selected from the group consisting of: 0.1 milligram per kilogram of subject weight (mg/kg), 0.3 mg/kg, 1.0 mg/kg, 1.5 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, and 24 mg/kg.

37. The method of claim 36, wherein the binding protein is administered at a dose selected from the group consisting of: about 0.5 mg/kg, 1 mg/kg, 1.5 mg/kg, and 3 mg/kg.

38. The method of claim 21, wherein the binding protein is administered at a dose selected from the group consisting of: about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, about 325-350 mg, 350-375 mg, or 375-400 mg of the binding protein.

39. The method of claim 38, wherein the dose comprises at least about 60 mg, about 120 mg, about 200 mg, or about 240 mg.

40. The method of claim 21, wherein the binding protein is administered in a single dose.

41. The method of claim 21, wherein the binding protein is administered in multiple doses.

42-83. (canceled)
